(19) United States
(12) Patent Application Publication
(10) Pub. No.: US 2024/0189407 A1
(43) Pub. Date: Jun. 13, 2024

(54) MODIFIED VIRUS-LIKE PARTICLES OF BACTERIOPHAGE AP205

(71) Applicant: SAIBA AG, Pfäffikon (CH)

(72) Inventors: Kaspars TARS, Jaunolaine (LV); Ilva LIEKNINA, Riga (LV); Darja CERNOVA, Riga (LV)

(21) Appl. No.: 18/554,935

(22) PCT Filed: Apr. 11, 2022
(86) PCT No.: PCT/EP2022/059646

§ 371 Date: Oct. 11, 2023

(30) Foreign Application Priority Data
Apr. 12, 2021 (EP) 21167817.2

(51) Int. Cl. A61K 39/00; A61K 39/015; A61K 39/02; A61K 39/12; A61K 39/35; A61P 31/14; A61P 37/08; C12N 7/00

(52) U.S. Cl. CPC A61K 39/00114; A61K 39/001134; A61K 39/001138; A61K 39/015; A61K 39/0225; A61K 39/12; A61K 39/35; A61P 31/14; A61P 37/08; C12N 7/00; C12N 2710/12034; C12N 2760/18534; C12N 2760/18571; C12N 2770/20034; C12N 2770/20071; C12N 2770/24134; C12N 2795/18022; C12N 2795/18023

(57) ABSTRACT

The present invention relates to a modified virus-like particle of RNA bacteriophage AP205 (AP205 VLP) comprising AP205 coat protein dimers to which antigenic polypeptides are fused at the N-terminus and/or at the C-terminus. The modified AP205 VLPs can be used as a platform, in particular for vaccine development, in generating immune responses against a variety of antigens.

The present invention relates to a modified virus-like particle of RNA bacteriophage AP205 (AP205 VLP) comprising AP205 coat protein dimers to which antigenic polypeptides are fused at the N-terminus and/or at the C-terminus. The modified AP205 VLPs can be used as a platform, in particular for vaccine development, in generating immune responses against a variety of antigens.

### RELATED ART

Virus-like particles (VLPs) are shells of viruses, devoid of viral genome, but morphologically and immunologically similar to the respective viruses. Therefore, VLPs can be used to create efficient and safe vaccines against the respective viruses, the best-known examples being Hepatitis B vaccine, composed of Hepatitis B virus S antigen VLPs and vaccines against cervical cancer, which are composed of human papilloma virus VLPs. Physically, VLPs are composed of multiple copies of viral coat protein, forming either icosahedral or rod-like nanoparticles. Recombinant VLPs can be produced by overproducing the respective coat protein gene in bacterial, yeast or other expression systems. Classical vaccines are normally composed of inactivated or attenuated pathogens, which still contain the genetic information. In contrast, VLPs do not contain any information about the genome of pathogens, therefore they are considered to be a safer alternative to classical vaccines. VLP based technology is not limited to creation of vaccines against the virus of VLP origin, but, in fact, VLPs can be used to generate immune responses against heterologous antigens (Frietze K M et al., Curr Opin Virol. 2016, 18: 44-49; Aves K L et al. Viruses 2020, 12, 185). Among them, VLPs of single stranded RNA bacteriophages like MS2 and Qβ have been used in construction of vaccines (Tars K, 2020 In: Witzany G. (eds) Biocommunication of Phages. Springer, Cham.). Most frequently, two technologies are used—chemical coupling or genetic fusion. Recombinant fusion proteins of foreign antigens and viral structural proteins often, however, fail to assemble into VLPs due to folding, formation of insoluble products and/or assembly problems of the obtained fusion protein. Moreover, genetic fusions are generally limited to small peptide antigens that do not inhibit the required particle assembly. By way of example, for RNA bacteriophage MS2 VLPs, foreign peptides inserted within the virus coat proteins are rarely compatible with protein folding and VLP assembly (Caldeira J C et al., J Nanobiotechnology. 2011; 9:22; Peabody D S, Arch Biochem Biophys. 1997; 347:85-92; Plevka P et al., Protein Sci. 2009; 18:1653-1661; Peabody D S et al., J Mol Biol. 2008; 380:252-263; WO2008024427A2; US20090054246A1; O'Rourke et al., Current Opinion in Virology 2015, 11:76-82). Therefore, the success of genetic fusion is difficult to predict, let alone that the successful identification of foreign antigen insertion sites in VLPs is still a major challenge (Frietze K M et al., Curr Opin Virol. 2016, 18: 44-49; Aves K L et al. Viruses 2020, 12, 185; and references cited therein).

RNA-bacteriophage AP205 infects Acinetobacter bacteria and is very distantly related to other RNA-bacteriophages such as MS2 or Qβ. Sequence alignment of its coat protein with other RNA bacteriophage coat proteins revealed that only 5 amino acids are conserved (Shishovs et al., J Mol Biol 2016, 428:4267-4279). The assembled AP205 VLPs have been described to be stable and suggested as vaccine platform and even found to tolerate fusions to the N or the C terminus (WO04/007538, WO2006/032674, Tissot A C et al., PLoS One 5:e9809, WO2016/112921).

Despite these achievements, there is still a need for a robust and versatile VLP platform, in particular for vaccine development, which is able to generate immune responses against a variety of antigens, and in particular, to generate immune responses against desired antigens and hereby irrespective and independent of the length of said antigens.

### SUMMARY OF THE INVENTION

It has been surprisingly found that the modified virus-like particle of RNA bacteriophage AP205 (AP205 VLP) of the present invention comprising AP205 coat protein dimers not only allows the fusion of antigens to the N or C-terminus of said AP205 coat protein dimers irrespective of the size of the antigens, but, furthermore, the inventive modified AP205 VLP comprising AP205 coat protein dimers also allows fusion of antigens to both termini of the AP205 coat protein dimers without affecting the ability to form VLPs. Thus, the present invention provides a modified virus-like particle of RNA bacteriophage AP205 (AP205 VLP) comprising AP205 coat protein dimers to which antigenic polypeptides are fused at the N- and/or at the C-terminus. The inventive modified AP205 VLPs can, thus, be used as a platform, in particular for vaccine development, in generating immune responses against a variety of antigens and even against different antigens presented on the same VLP. In particular, said provided AP205 coat protein dimers represents a tool to create recombinant VLPs with exposed large antigens via genetic fusion of said antigens to the AP205 coat protein dimers and hereby at either the N-terminus and/or the C-terminus of said AP205 coat protein dimers. In addition, said inventive AP205 coat protein dimers, also named herein coat protein tandem dimer of bacteriophage AP205 or tandem dimer of AP205, allow genetic fusion with long antigen sequences without compromising VLP integrity and stability.

In particular, the inventors have found that attempts to insert and fuse the 213 amino acid long CspZ protein from Borrelia burgdorferi to the N- or the C-terminal part of the single coat protein of AP205 failed and did not form VLPs but insoluble products in both cases as schematically represented in FIG. 2. On the other hand, the resulting fusion proteins of the AP205 coat protein dimers of the present invention with said CspZ protein from Borrelia burgdorferi lead to intact modified virus-like particles in both cases, i.e. either when said CspZ antigen was fused at the N-terminus or when said CspZ antigen was fused to the C-terminus of the AP205 coat protein dimer. Furthermore, and besides the bacterial antigen of CspZ protein from Borrelia burgdorferi having a length of 213 amino acids, the inventors further achieved in providing modified AP205 VLPs in accordance with the present invention for antigens of viral and mammalian origin, including ectodomain III (ED3) from E protein of Dengue fever virus serotype 1 having a length of 117 amino acids, the RBM domain of SARS-CoV-2 having a length of 72 amino acids, and of canine interleukin-33 (cIL-33) with a length of 163 amino acids. These modified VLPs are useful for vaccination against Lyme borreliosis, Dengue fever and canine atopic dermatitis, respectively. In summary, the present invention demonstrates that VLPs of AP205 coat protein dimers in accordance with the present invention provide a universal VLP platform, able to tolerate insertions of a wide variety of antigens, in particular of large antigens, by genetic fusion to either N- and/or C-terminus of said AP205 coat protein dimers.

Thus, in a first aspect, the present invention provides a modified virus-like particle of RNA bacteriophage AP205 (AP205 VLP) comprising one or more fusion proteins, wherein said fusion protein comprises, preferably consists of,
(i) an AP205 coat protein dimer, wherein said AP205 coat protein dimer comprises a first AP205 polypeptide and a second AP205 polypeptide, wherein said first AP205 polypeptide is fused at its C-terminus either directly or via an amino acid spacer to the N-terminus of said second AP205 polypeptide, and wherein said first and said second AP205 polypeptide independently comprises
   (a) an amino acid sequence of a coat protein of RNA bacteriophage AP205, or
   (b) a mutated amino acid sequence, wherein said mutated amino acid sequence (b) and said amino acid sequence of said coat protein of RNA bacteriophage AP205 (a), have a sequence identity of at least 90%, preferably of at least 95%, further preferably of at least 98% and again more preferably of at least 99%; and
(ii) an antigenic polypeptide, wherein said antigenic polypeptide is fused to the N-terminus and/or the C-terminus of said AP205 coat protein dimer either directly or via an amino acid linker.

In a further aspect, the present invention provides the modified AP205 VLP of the present invention for use as a medicament.

In again a further aspect, the present invention provides a pharmaceutical composition comprising (a) the AP205 VLP of the present invention, and (b) a pharmaceutically acceptable carrier, diluent and/or excipient.

In a further aspect, the present invention provides the modified AP205 VLP or the pharmaceutical composition of the present invention for use in a method of immunization an animal or a human, comprising administering the modified AP205 VLP or the pharmaceutical composition to said animal or human.

In another aspect, the present invention provides the modified AP205 VLP or the pharmaceutical composition of the present invention for use in a method of treating or preventing a disease or disorder in an animal or human, comprising administering the modified AP205 VLP or the pharmaceutical composition to said animal or human.

In another aspect, the present invention provides a modified virus-like particle of RNA bacteriophage AP205 (AP205 VLP) comprising one or more AP205 coat protein dimer, wherein said AP205 coat protein dimer comprises a first AP205 polypeptide and a second AP205 polypeptide, wherein said first AP205 polypeptide is fused at its C-terminus either directly or via an amino acid spacer to the N-terminus of said second AP205 polypeptide, and wherein said first and said second AP205 polypeptide independently comprises
(a) an amino acid sequence of a coat protein of RNA bacteriophage AP205, or
(b) a mutated amino acid sequence, wherein said mutated amino acid sequence (b) and said amino acid sequence of said coat protein of RNA bacteriophage AP205 (a), have a sequence identity of at least 90%, preferably of at least 95%, further preferably of at least 98% and again more preferably of at least 99%.

Further aspects and embodiments of the present invention will be become apparent as this description continues.

### DESCRIPTION OF FIGURES

FIG. 1: AP205 tandem dimer coat protein. Two AP205 coat protein genes are fused together, resulting in production of tandem coat protein dimer, where C-terminus of one monomer is covalently joined to the N-terminus of other monomer. The covalent dimer is depicted in rainbow colour from blue (N-terminus) to red (C-terminus). Electron micrograph of AP205 tandem dimer VLPs is shown as well.

FIG. 2: Tolerance of VLPs of AP205 tandem dimer. Single AP205 coat protein fused with long antigen such as CspZ either at the N-terminus or at the C-terminus did not lead to VLPs but to insoluble products, whereas the AP205 tandem dimer VLPs tolerate C- or N-terminal fusions of such long antigens.

FIG. 3A: Production and solubility of CspZ-AP205TD. Lane 1—cell lysate, lane 2—soluble fraction, lane 3—insoluble fraction. Position of CspZ-AP205TD protein is indicated with an arrow.

FIG. 3B: Production and solubility of AP205TD-CspZ. Lane 1—cell lysate, lane 2—soluble fraction, lane 3—insoluble fraction. Position of AP205TD-CspZ protein is indicated with an arrow.

FIG. 4A: Purification of CspZ-AP205TD by gel-filtration. Peak fractions are loaded on SDS-PAGE gel. Position of CspZ-AP205TD protein is indicated with an arrow.

FIG. 4B: Purification of CspZ-AP205TD by gel-filtration. Peak fractions are loaded on native agarose gel and stained both with ethidium bromide for the RNA content of VLPs (left panel) and coomassie blue for the protein content (right panel).

FIG. 5: Electron micrograph of purified CspZ-AP205TD VLPs.

FIG. 6A: Purification of AP205TD-CspZ by gel-filtration. Peak fractions are loaded on SDS-PAGE gel. Position of AP205TD-CspZ protein is indicated with an arrow.

FIG. 6B: Peak fractions are loaded on native agarose gel and stained both with ethidium bromide for the RNA content of VLPs (left panel) and coomassie blue for the protein content (right panel).

FIG. 7: Electron micrograph of purified AP205TD-CspZ VLPs.

FIG. 8A: Production and solubility of ED3-AP205TD. Lane 1—cell lysate, lane 2—soluble fraction, lane 3—insoluble fraction. Position of ED3-AP205TD protein is indicated with an arrow.

FIG. 8B: Refolding of ED3-AP205TD. Lane 1—insoluble fraction (as in lane 3 of panel A), solubilized with 8 M urea, lane 2—soluble fraction after refolding, lane 3—insoluble fraction after refolding. Position of ED3-AP205TD protein is indicated with an arrow.

FIG. 9A: Purification of ED3-AP205TD by gel-filtration. Peak fractions are loaded on SDS-PAGE gel. Position of ED3-AP205TD protein is indicated with an arrow.

FIG. 9B: Purification of ED3-AP205TD by gel-filtration. Peak fractions are loaded on native agarose gel and stained both with ethidium bromide for the RNA content of VLPs (left panel) and coomassie blue for the protein content (right panel).

FIG. 10: Electron micrograph of purified ED3-AP205TD VLPs.

FIG. 11: Production and solubility of AP205TD-cIL33. Lane 1—cell lysate, lane 2—soluble fraction, lane 3—insoluble fraction. Position of the ˜47 kDa AP205TD-cIL33 protein is indicated with an arrow.

FIG. 12A: Purification of AP205TD-cIL33 by gel-filtration. Peak fractions are loaded on SDS-PAGE gel. Position of AP205TD-cIL33 protein is indicated with an arrow.

FIG. 12B: Purification of AP205TD-cIL33 by gel-filtration. Peak fractions are loaded on native agarose gel and stained both with ethidium bromide for the RNA content of VLPs (left panel) and coomassie blue for the protein content (right panel).

FIG. 13: Electron micrograph of purified AP205TD-cIL33 VLPs.

FIG. 14: Immunization of mice with AP205TD-cIL33 VLPs to generate binding and neutralizing antibodies. The upper panel shows IL33-binding IgG antibody titers measured by ELISA and the lower panel shows IL33-neutralizing antibody titers determined by an in vitro cellular assay.

FIG. 15A: Schematic representation of AP205-RBM fusion construct.

FIG. 15B: 12% SDS-PAGE for AP205-RBM expression in E. coli showing soluble (S) and insoluble (P) fractions.

FIG. 15C: 12% SDS-PAGE showing the purified AP205-RBM vaccine of ˜37.4 kDa.

FIG. 15D: Electron Microscopy of negatively stained AP205-RBM VLPs showing a size of ˜30 nm, scale bar 200 nm

FIG. 16A: Vaccination with AP205-RBM vaccine induces high titer of RBD and Spike-specific IgG Abs. RBD-specific IgG titer for the groups vaccinated with AP205 control and AP205-RBM vaccine on days 14, 21, 35 and 49, measured by ELISA (OD50 in D: given as reciprocal dilution values), three-fold serial serum dilution was used starting from 1:20.

FIG. 16B: Spike-specific IgG titer for the groups vaccinated with AP205 control and AP205-RBM vaccine on days 14, 21, 35 and 49, measured by ELISA (OD50 in E: given as reciprocal dilution values), three-fold serial serum dilution was used starting from 1:20

FIG. 17A: The AP205-RBM vaccine-candidate induces antibodies neutralizing SARS-CoV2. Neutralization titer of the induced antibodies using a SARS-CoV-2 pseudo-typed VSV virus assay.

FIG. 17B: Neutralization titer of the induced antibodies using CPE method and 100 TCID50 of SARS-CoV-2/ABS/NL20 virus.

FIG. 18. Production and solubility of np12-AP205TD-cp12. M—protein ladder (molecular weights as indicated), lane 1—cell lysate before the induction with IPTG, lane 2-cell lysate after the induction with IPTG, lane 3—soluble fraction of the cell lysate, lane 4—insoluble fraction. Position of np12-AP205TD-cp12 protein is indicated with an arrow.

FIG. 19A. Purification of np12-AP205TD-cp12 by gel-filtration. Analysis of peak fractions on SDS-PAGE gel. Position of np12-AP205TD-cp12 protein is indicated with an arrow.

FIG. 19B: Purification of np12-AP205TD-cp12 by gel-filtration. Analysis of peak fractions on native agarose gel, stained with ethidium bromide for the RNA content of VLPs (left panel) and coomassie blue for the protein content (right panel).

FIG. 20A: Purification of np12-AP205TD-cp12 by ion exchange chromatography. Analysis of peak fractions on SDS-PAGE gel. Position of np12-AP205TD-cp12 protein is indicated with an arrow.

FIG. 20B: Purification of np12-AP205TD-cp12 by ion exchange chromatography. Analysis of peak fractions on native agarose gel, stained with ethidium bromide for the RNA content of VLPs (left panel) and coomassie blue for the protein content (right panel).

FIG. 21: Electron micrograph of purified np12-AP205TD-cp12 VLPs.

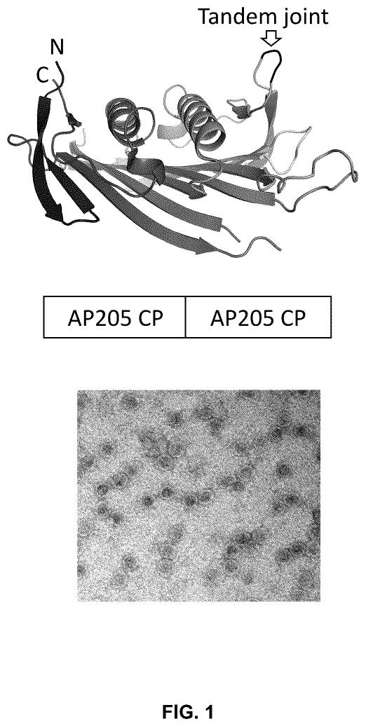

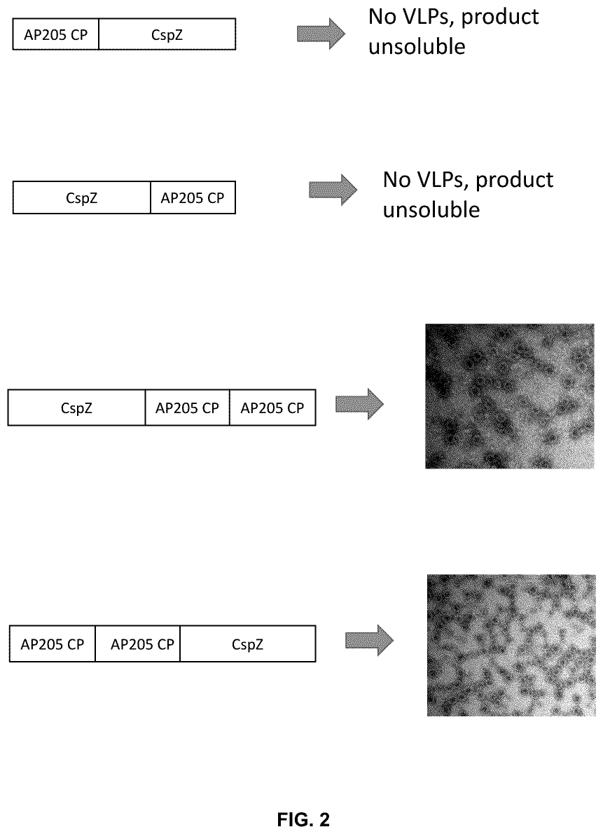

### DETAILED DESCRIPTION OF THE INVENTION

Unless defined otherwise, all technical and scientific terms used herein have the same meanings as commonly understood by one of ordinary skill in the art to which this invention belongs. The herein described and disclosed embodiments, preferred embodiments and very preferred embodiments should apply to all aspects and other embodiments, preferred embodiments and very preferred embodiments irrespective of whether is specifically again referred to or its repetition is avoided for the sake of conciseness. The articles “a” and “an”, as used herein, refer to one or to more than one (i.e., to at least one) of the grammatical object of the article. The term “or”, as used herein, should be understood to mean “and/or”, unless the context clearly indicates otherwise. As used herein, the terms “about” when referring to any numerical value are intended to mean a value of +10% of the stated value. In a preferred embodiment, said “about” when referring to any numerical value are intended to mean a value of 55% of the stated value. In another preferred embodiment, said “about” when referring to any numerical value are intended to mean a value of ±3% of the stated value.

Virus-like particle (VLP): The term “virus-like particle (VLP)” as used herein, refers to a non-replicative or non-infectious, preferably a non-replicative and non-infectious virus particle, or refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. A virus-like particle in accordance with the invention is non-replicative and non-infectious since it lacks all or part of the viral genome or genome function. A virus-like particle in accordance with the invention may contain nucleic acid distinct from their genome. Recombinantly produced virus-like particles typically contain host cell derived RNA.

Modified virus-like particle of RNA bacteriophage AP205 (modified AP205 VLP): The term “modified virus-like particle of RNA bacteriophage AP205 (modified AP205 VLP)” refers to a virus-like particle comprising at least one, typically and preferably about 90, further preferably exactly 90, AP205 coat protein dimers in accordance with the present invention. Typically and preferably, modified AP205 VLPs resemble the structure of the capsid of RNA bacteriophage AP205. Modified AP205 VLPs are non-replicative and/or non-infectious, and lack at least the gene or genes encoding for the replication machinery of RNA bacteriophage AP205, and typically also lack the gene or genes encoding the protein or proteins responsible for viral attachment to or entry into the host. This definition includes also modified virus-like particles in which the aforementioned gene or genes are still present but inactive. Preferably, non-replicative and/or non-infectious modified virus-like particles are obtained by recombinant gene technology and typically and preferably do not comprise the viral genome.

Polypeptide: The term “polypeptide” as used herein refers to a polymer composed of amino acid monomers which are linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. The term “polypeptide” as used herein should also refer, typically and preferably to a polypeptide as defined before and encompassing modifications such as post-translational modifications, including but not limited to glycosylations. In a preferred embodiment, said term “polypeptide” as used herein should refer to a polypeptide as defined before and not encompassing modifications such as post-translational modifications such as glycosylations. In particular, for said biologically active peptides, said modifications such as said glycosylations can occur even in vivo thereafter, for example, by bacteria.

AP205 polypeptide: The term “AP205 polypeptide” as used herein—be it for a “first AP205 polypeptide” or “second AP205 polypeptide” independently—refers to a polypeptide comprising or preferably consisting of: (i) an amino acid sequence of a coat protein of RNA bacteriophage AP205, or (ii) a mutated amino acid sequence, wherein said mutated amino acid sequence and said amino acid sequence of said coat protein of CMV, have a sequence identity of at least 90%, preferably of at least 95%, further preferably of at least 98% and again more preferably of at least 99%. Typically and preferably, the AP205 coat protein dimer comprising a first AP205 polypeptide and a second AP205 polypeptide is capable of forming a virus-like particle of AP205 upon expression by self-assembly.

Coat protein (CP) of RNA bacteriophage AP205: The term “coat protein (CP) of RNA bacteriophage AP205 (or, in short and interchangeably used, coat protein of AP205)”, as used herein, refers to a coat protein of the RNA bacteriophage AP205 which occurs in nature or said amino acid sequence of said coat protein wherein the first methionine is cleaved. The sequences of said coat proteins (CPs) of AP205 are described in and retrievable from the known databases such as Genbank, www.dpvweb.net, or www.ncbi.nlm.nih.gov/protein/. Specific and preferred examples CPs of AP205, and mutated amino acid sequences thereof, are described in Klovins, J., et al., 2002, J. Gen. Virol. 83:1523-33 and WO2006/032674, the disclosures of which are explicitly incorporated herein by way of reference. In a preferred embodiment of the present invention, said coat protein of AP205 consists of a length of about 131 amino acids, typically and preferably said coat protein of AP205 consists of a length of 128 to 133 amino acids. Very preferred examples and embodiments of AP205 coat proteins are provided in SEQ ID NO:25 and SEQ ID NO:26.

Recombinant polypeptide: In the context of the invention the term “recombinant” when used in the context of a polypeptide refers to a polypeptide which is obtained by a process which comprises at least one step of recombinant DNA technology. Typically and preferably, a recombinant polypeptide is produced in a prokaryotic expression system. It is apparent for the artisan that recombinantly produced polypeptides which are expressed in a prokaryotic expression system such as E. coli may comprise an N-terminal methionine residue. The N-terminal methionine residue is typically cleaved off the recombinant polypeptide in the expression host during the maturation of the recombinant polypeptide. However, the cleavage of the N-terminal methionine may be incomplete. Thus, a preparation of a recombinant polypeptide may comprise a mixture of otherwise identical polypeptides with and without an N-terminal methionine residue. Typically and preferably, a preparation of a recombinant polypeptide comprises less than 10%, more preferably less than 5%, and still more preferably less than 1% recombinant polypeptide with an N-terminal methionine residue.

Recombinant modified virus-like particle: In the context of the invention the term “recombinant modified virus-like particle” refers to a modified virus-like particle (VLP) which is obtained by a process which comprises at least one step of recombinant DNA technology.

Mutated amino acid sequence: The term “mutated amino acid sequence” refers to an amino acid sequence which is obtained by introducing a defined set of mutations into an amino acid sequence to be mutated. In the context of the invention, said amino acid sequence to be mutated typically and preferably is an amino acid sequence of a coat protein of AP205. Thus, a mutated amino acid sequence differs from an amino acid sequence of a coat protein of AP205 in at least one amino acid residue, wherein said mutated amino acid sequence and said amino acid sequence of a coat protein of AP205 have a sequence identity of at least 90%. Typically and preferably said mutated amino acid sequence and said amino acid sequence of a coat protein of AP205 have a sequence identity of at least 91%, 92%, 93% 94%, 95%, 96%, 97%, 98%, or 99%. Preferably, said mutated amino acid sequence and said sequence of a coat protein of AP205 differ in at most 11, 10, 9, 8, 7, 6, 4, 3, 2, or 1 amino acid residues, wherein further preferably said difference is selected from insertion, deletion and amino acid exchange and a combination thereof. Preferably, the mutated amino acid sequence differs from an amino acid sequence of a coat protein of CMV in three, two or one amino acid, wherein preferably said difference is an amino acid exchange, deletion or addition, and a combination thereof.

Sequence identity: The sequence identity of two given amino acid sequences is determined based on an alignment of both sequences. Algorithms for the determination of sequence identity are available to the artisan. Preferably, the sequence identity of two amino acid sequences is determined using publicly available computer homology programs such as the “BLAST” program (http://blast.ncbi.nlm.nih.gov/Blast.cgi) or the “CLUSTALW” (http://www.genome.jp/tools/clustalw/), and hereby preferably by the “BLAST” program provided on the NCBI homepage at http://blast.ncbi.nlm.nih.gov/Blast.cgi, using the default settings provided therein. Typical and preferred standard settings are: expect threshold: 10; word size: 3; max matches in a query range: 0; matrix: BLOSUM62; gap costs: existence 11, extension 1; compositional adjustments: conditional compositional score matrix adjustment.

Amino acid exchange: The term amino acid exchange refers to the exchange of a given amino acid residue in an amino acid sequence by any other amino acid residue having a different chemical structure, preferably by another proteinogenic amino acid residue. Thus, in contrast to insertion or deletion of an amino acid, the amino acid exchange does not change the total number of amino acids of said amino acid sequence. In case of an amino acid exchange within the present invention and thus, also referred to as an amino acid substitution, conservative amino acid substitutions are preferred. Conservative amino acid substitutions, as understood by a skilled person in the art, include, and typically and preferably consist of isosteric substitutions, substitutions where the charged, polar, aromatic, aliphatic or hydrophobic nature of the amino acid is maintained. Typical conservative substitutions are substitutions between amino acids within one of the following groups: Gly, Ala; Val, Ile, Leu; Asp, Glu; Asn, Gln; Ser, Thr, Cys; Lys, Arg; and Phe and Tyr.

Antigenic polypeptide: As used herein, the term “antigenic polypeptide” refers to a molecule capable of being bound by an antibody or a T-cell receptor (TCR) if presented by MHC molecules. An antigenic polypeptide is additionally capable of being recognized by the immune system and/or being capable of inducing a humoral immune response and/or cellular immune response leading to the activation of B- and/or T-lymphocytes. An antigenic polypeptide can have one or more epitopes (B- and T-epitopes). Antigenic polypeptides as used herein may also be mixtures of several individual antigenic polypeptides. The inventive fusion proteins which are forming the inventive modified AP205 VLPs comprise the antigenic polypeptide. In case, the inventive fusion proteins which are forming the inventive modified AP205 VLPs comprise an antigenic polypeptide fused to the N-terminus of said AP205 coat protein dimer and an antigenic polypeptide fused to the C-terminus of said AP205 coat protein dimer in accordance with the present invention, the terms “first antigenic polypeptide” and “second antigenic polypeptide” are also used herein, preferably to distinguish the antigenic polypeptide fused to the N-terminus of said AP205 coat protein dimer and the antigenic polypeptide fused to the C-terminus of said AP205 coat protein dimer. However, all antigenic polypeptides mentioned herein as preferred antigenic polypeptides are understood to equally read on first antigenic polypeptides and/or second antigenic polypeptides irrespective whether it is explicitly stated as such. For the sake of conciseness such repetitions have been omitted.

Epitope: The term epitope refers to continuous or discontinuous portions of an antigenic polypeptide, wherein said portions can be specifically bound by an antibody or by a T-cell receptor within the context of an MHC molecule. With respect to antibodies, specific binding excludes non-specific binding but does not necessarily exclude cross-reactivity. An epitope typically comprise 5-20 amino acids in a spatial conformation which is unique to the antigenic site.

Receptor binding domain: The term “protein domain” and “receptor binding domain” as used herein, refers to parts of proteins that either occur alone or together with partner domains on the same protein chain. Most domains correspond to tertiary structure elements and are able to fold independently. All domains exhibit evolutionary conservation, and many either perform specific functions or contribute in a specific way to the function of their proteins (Forslund S K et al, Methods Mol Biol. (2019) 1910:469-504). Viral structural proteins, such as Coronavirus S proteins, can contain several functional domains, which are necessary for the cell infection process. One such domain in Coronavirus S protein is the receptor binding domain (RBD) which binds to corresponding cell receptor.

Receptor binding motif: The term “receptor binding motif (RBM)”, as used herein, is a part of receptor binding domain and represent a linear amino acid sequence and/or a 3D structure located on outer surface of the virus and making direct contact with target cell receptors (Sobhy H, Proteomes (2016) 4(1): 3). For Coronaviruses, the amino acids sequences of RBMs have low homology due to different target cellular receptors. For SARS-CoV2, 16 amino acids of RBM make direct contacts with human ACE2 receptor (Lan et al., Structure of the SARS-CoV-2 spike receptor-binding domain bound to the ACE2 receptor, Nature, 2020, 581, 215-220).

Adjuvant: The term “adjuvant” as used herein refers to non-specific stimulators of the immune response or substances that allow generation of a depot in the host which when combined with the vaccine and pharmaceutical composition, respectively, of the present invention may provide for an even more enhanced immune response. Preferred adjuvants are complete and incomplete Freund's adjuvant, aluminum containing adjuvant, preferably aluminum hydroxide, and modified muramyldipeptide. Further preferred adjuvants are mineral gels such as aluminum hydroxide, surface active substances such as lyso lecithin, pluronic polyols, polyanions, peptides, oil emulsions, keyhole limpet hemocyanins, dinitrophenol, and human adjuvants such as BCG (bacille Calmette Guerin) and Corynebacterium parvum. Such adjuvants are also well known in the art. Further adjuvants that can be administered with the compositions of the invention include, but are not limited to, Monophosphoryl lipid immunomodulator, AdjuVax 100a, QS-21, QS-18, CRL1005, Aluminum salts (Alum), MF-59, OM-174, OM-197, OM-294, and Virosomal adjuvant technology. The adjuvants may also comprise mixtures of these substances. Virus-like particles have been generally described as an adjuvant. However, the term “adjuvant”, as used within the context of this application, refers to an adjuvant not being the inventive modified virus-like particle. Rather “adjuvant” relates to an additional, distinct component of the inventive compositions, vaccines or pharmaceutical compositions.

Amino acid linker: The term “amino acid linker” as used herein, refers to a linker consisting exclusively of amino acid residues. The amino acid residues of the amino acid linker are composed of naturally occurring amino acids or unnatural amino acids known in the art, all-L or all-D or mixtures thereof. The amino acid residues of the amino acid linker are preferably naturally occurring amino acids, all-L or all-D or mixtures thereof.

Amino acid spacer: The term “amino acid spacer” as used herein, refers to a linkage consisting exclusively of amino acid residues linking the first AP205 polypeptide with the second AP205 polypeptide of the present invention. The amino acid residues of the amino acid spacer are composed of naturally occurring amino acids or unnatural amino acids known in the art, all-L or all-D or mixtures thereof. The amino acid residues of the amino acid spacer are preferably naturally occurring amino acids, all-L or all-D or mixtures thereof.

GS-linker: The term “GS-linker”, as used herein refers to a linker solely consisting of glycine and serine amino acid residues. The GS-linker in accordance with the present invention comprise at least one glycine and at least one serine residue. Typically and preferably, the GS-linker in accordance with the present invention has a length of at most 50 amino acids, and typically and further preferably, the GS-linker in accordance with the present invention has a length of at most 30 amino acids, further preferably of at most 15 amino acids.

Animal: The term “animal”, as used herein and being the subject in need of treatment or prevention with the inventive modified AP205 VLPs, may be an animal (e.g., a non human animal), a vertebrate animal, a mammal, a rodent (e.g., a guinea pig, a hamster), a canine (e.g., a dog), a feline (e.g., a cat), a porcine (e.g., a pig), an equine (e.g., a horse), a primate, or a human. In the context of this invention, it is particularly envisaged that animals are to be treated which are economically, agronomically or scientifically important. Scientifically important organisms include, but are not limited to, mice, rats, and rabbits. Non-limiting examples of agronomically important animals are sheep, cattle and pigs, while, for example, cats, dogs and horses may be considered as economically important animals. Preferably, the subject is a mammal; more preferably, the subject is a human or a non-human mammal (such as, e.g., a dog, a cat, a horse, a sheep, cattle, or a pig).

Effective amount: As used herein, the term “effective amount” refers to an amount necessary or sufficient to realize a desired biologic effect. An effective amount of the composition, or alternatively the pharmaceutical composition, would be the amount that achieves this selected result, and such an amount could be determined as a matter of routine by a person skilled in the art. The effective amount can vary depending on the particular composition being administered and the size of the subject. One of ordinary skill in the art can empirically determine the effective amount of a particular composition of the present invention without necessitating undue experimentation.

Therapeutically effective amount: As used herein, the term “therapeutically effective amount” refers to an amount that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. For example, in case of a cancer, the therapeutically effective amount may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer.

Treatment: As used herein, the terms “treatment”, “treat”, “treated” or “treating” refer to prophylaxis and/or therapy. In one embodiment, the terms “treatment”, “treat”, “treated” or “treating” refer to a therapeutic treatment. In another embodiment, the terms “treatment”, “treat”, “treated” or “treating” refer to a prophylactic treatment

In a first aspect, the present invention provides a modified virus-like particle of RNA bacteriophage AP205 (AP205 VLP) comprising one or more fusion proteins, wherein said fusion protein comprises, preferably consists of,
(i) an AP205 coat protein dimer, wherein said AP205 coat protein dimer comprises a first AP205 polypeptide and a second AP205 polypeptide, wherein said first AP205 polypeptide is fused at its C-terminus either directly or via an amino acid spacer to the N-terminus of said second AP205 polypeptide, and wherein said first and said second AP205 polypeptide independently comprises
   (a) an amino acid sequence of a coat protein of RNA bacteriophage AP205, or
   (b) a mutated amino acid sequence, wherein said mutated amino acid sequence (b) and said amino acid sequence of said coat protein of RNA bacteriophage AP205 (a), have a sequence identity of at least 90%, preferably of at least 95%, further preferably of at least 98% and again more preferably of at least 99%; and
(ii) an antigenic polypeptide, wherein said antigenic polypeptide is fused to the N-terminus and/or the C-terminus of said AP205 coat protein dimer either directly or via an amino acid linker.

In another aspect, the present invention provides a modified virus-like particle of RNA bacteriophage AP205 (AP205 VLP) comprising one or more fusion proteins, wherein said fusion protein comprises, preferably consists of,
(i) an AP205 coat protein dimer, wherein said AP205 coat protein dimer comprises a first AP205 polypeptide and a second AP205 polypeptide, wherein said first AP205 polypeptide is fused at its C-terminus either directly or via an amino acid spacer to the N-terminus of said second AP205 polypeptide, and wherein said first and said second AP205 polypeptide independently comprises
   (a) an amino acid sequence of a coat protein of RNA bacteriophage AP205, or
   (b) a mutated amino acid sequence, wherein said mutated amino acid sequence (b) and said amino acid sequence of said coat protein of RNA bacteriophage AP205 (a), have a sequence identity of at least 90%, preferably of at least 95%, further preferably of at least 98% and again more preferably of at least 99%; and
(ii) (1) an antigenic polypeptide fused directly or via an amino acid linker to the N-terminus of said AP205 coat protein dimer;
   (2) an antigenic polypeptide fused directly or via an amino acid linker to the C-terminus of said AP205 coat protein dimer; or
   (3) a first antigenic polypeptide fused directly or via an amino acid linker to the N-terminus of said AP205 coat protein dimer and a second antigenic polypeptide fused directly or via an amino acid linker to the C-terminus of said AP205 coat protein dimer.

In another aspect and preferred embodiment, the present invention provides a modified virus-like particle of RNA bacteriophage AP205 (AP205 VLP) comprising one or more fusion proteins, wherein said fusion protein comprises, preferably consists of,
(i) an AP205 coat protein dimer, wherein said AP205 coat protein dimer comprises a first AP205 polypeptide and a second AP205 polypeptide, wherein said first AP205 polypeptide is fused at its C-terminus either directly or via an amino acid spacer to the N-terminus of said second AP205 polypeptide, and wherein said first and said second AP205 polypeptide independently comprises
   (a) an amino acid sequence of a coat protein of RNA bacteriophage AP205, or
   (b) a mutated amino acid sequence, wherein said mutated amino acid sequence (b) and said amino acid sequence of said coat protein of RNA bacteriophage AP205 (a), have a sequence identity of at least 90%, preferably of at least 95%, further preferably of at least 98% and again more preferably of at least 99%, and wherein preferably said AP205 coat protein dimer comprises, preferably consists of, the amino sequence of SEQ ID NO: 7 or an amino acid sequence having a sequence identity of at least 90%, preferably of at least 95%, further preferably of at least 98% and again more preferably of at least 99% with said SEQ ID NO: 7; and
(ii) (1) an antigenic polypeptide fused directly or via an amino acid linker to the N-terminus of said AP205 coat protein dimer, wherein said antigenic polypeptide has a length of at least 60 amino acids and at most 300 amino acids, and wherein preferably said antigenic polypeptide has a length of at least 70 amino acids and at most 300 amino acids;
   (2) an antigenic polypeptide fused directly or via an amino acid linker to the C-terminus of said AP205 coat protein dimer, wherein said antigenic polypeptide has a length of at least 60 amino acids and at most 300 amino acids, and wherein preferably said antigenic polypeptide has a length of at least 70 amino acids and at most 300 amino acids; or
   (3) a first antigenic polypeptide fused directly or via an amino acid linker to the N-terminus of said AP205 coat protein dimer and a second antigenic polypeptide fused directly or via an amino acid linker to the C-terminus of said AP205 coat protein dimer, wherein said first antigenic polypeptide and said second antigenic polypeptide independently has a length of at least 5 amino acids and at most 80 amino acids, and wherein preferably said first antigenic polypeptide and said second antigenic polypeptide independently has a length of at least 5 amino acids and at most 60 amino acids, again further preferably, wherein said first antigenic polypeptide and said second antigenic polypeptide independently has a length of at least 5 amino acids and at most 50 amino acids;
      and wherein preferably said modified AP205 VLP consists of said fusion proteins.

In a preferred embodiment, said first and said second AP205 polypeptide independently comprises (a) an amino acid sequence of a coat protein of RNA bacteriophage AP205, or (b) a mutated amino acid sequence, wherein said mutated amino acid sequence (b) and said amino acid sequence of a coat protein of RNA bacteriophage AP205 (a), have a sequence identity at least 95%. In a preferred embodiment, said first and said second AP205 polypeptide independently comprises (a) an amino acid sequence of a coat protein of RNA bacteriophage AP205, or (b) a mutated amino acid sequence, wherein said mutated amino acid sequence (b) and said amino acid sequence of a coat protein of RNA bacteriophage AP205 (a), have a sequence identity at least 98%. In a preferred embodiment, said first and said second AP205 polypeptide independently comprises (a) an amino acid sequence of a coat protein of RNA bacteriophage AP205, or (b) a mutated amino acid sequence, wherein said mutated amino acid sequence (b) and said amino acid sequence of a coat protein of RNA bacteriophage AP205 (a), have a sequence identity at least 99%.

In a preferred embodiment, said first and said second AP205 polypeptide independently comprises (a) an amino acid sequence of a coat protein of RNA bacteriophage AP205, or (b) a mutated amino acid sequence, wherein said mutated amino acid sequence (b) and said amino acid sequence of said coat protein of RNA bacteriophage AP205 (a) differ in at most 11, 10, 9, 8, 7, 6, 4, 3, 2, or 1 amino acid residues, wherein said differences are independently selected from insertion, deletion, amino acid exchange and a combination thereof. In a preferred embodiment, the mutated amino acid sequence differs from an amino acid sequence of a coat protein of RNA bacteriophage AP205 in 1, 2, 3, 4 or 5 amino acids, said differences are independently selected from insertion, deletion, amino acid exchange and a combination thereof. In a preferred embodiment, the mutated amino acid sequence differs from an amino acid sequence of a coat protein of RNA bacteriophage AP205 in 1, 2 or 3 amino acids, said differences are independently selected from insertion, deletion, amino acid exchange and a combination thereof. In a preferred embodiment, the mutated amino acid sequence differs from an amino acid sequence of a coat protein of RNA bacteriophage AP205 in 3 amino acids, said differences are independently selected from insertion, deletion, amino acid exchange and a combination thereof. In a preferred embodiment, the mutated amino acid sequence differs from an amino acid sequence of a coat protein of RNA bacteriophage AP205 in 2 amino acids, said differences are independently selected from insertion, deletion, amino acid exchange and a combination thereof. In a preferred embodiment, the mutated amino acid sequence differs from an amino acid sequence of a coat protein of RNA bacteriophage AP205 in 1 amino acid, said difference is selected from insertion, deletion, and amino acid exchange.

In a preferred embodiment, said first and said second AP205 polypeptide independently comprises (a) an amino acid sequence of a coat protein of RNA bacteriophage AP205.

In a preferred embodiment, said first and said second AP205 polypeptide independently comprises, preferably consists of, an amino acid sequence selected from any of the SEQ ID NO: 25 to 33 and an amino sequence having a sequence identity of at least 90%, preferably of at least 95%, further preferably of at least 98%, and again further preferably of at least 99%, with any of the SEQ ID NO: 25 to 33. In a preferred embodiment, said first and said second AP205 polypeptide independently comprises, preferably consists of, an amino acid sequence selected from any of the SEQ ID NO: 25 to 33. In a preferred embodiment, said first AP205 polypeptide comprises, preferably consists of, an amino acid sequence selected from any of the SEQ ID NO: 25, 26, 28, 29, 31 and 32. In a preferred embodiment, said first AP205 polypeptide comprises, preferably consists of, an amino acid sequence selected from any of the SEQ ID NO: 25, 28 and 31. In a very preferred embodiment, said first AP205 polypeptide comprises, preferably consists of, the amino acid sequence of SEQ ID NO: 25. In a preferred embodiment, said second AP205 polypeptide comprises, preferably consists of, an amino acid sequence selected from any of the SEQ ID NO: 26, 27, 29, 30, 32 and 33. In a preferred embodiment, said second AP205 polypeptide comprises, preferably consists of, an amino acid sequence selected from any of the SEQ ID NO: 26 and 27. In a very preferred embodiment, said second AP205 polypeptide comprises, preferably consists of, the amino acid sequence of SEQ ID NO: 26. In a very preferred embodiment, said second AP205 polypeptide comprises, preferably consists of, the amino acid sequence of SEQ ID NO: 27.

In a preferred embodiment, said first and said second AP205 polypeptide independently comprises (a) an amino acid sequence of a coat protein of RNA bacteriophage AP205, or (b) a mutated amino acid sequence, wherein said mutated amino acid sequence (b) and said amino acid sequence of a coat protein of RNA bacteriophage AP205 (a), have a sequence identity at least 90%, wherein said coat protein of RNA bacteriophage AP205 comprises, preferably consists of, the amino acid sequence of SEQ ID NO: 26. In a preferred embodiment, said first and said second AP205 polypeptide independently comprises (a) an amino acid sequence of a coat protein of RNA bacteriophage AP205, or (b) a mutated amino acid sequence, wherein said mutated amino acid sequence (b) and said amino acid sequence of a coat protein of RNA bacteriophage AP205 (a), have a sequence identity at least 95%, wherein said coat protein of RNA bacteriophage AP205 comprises, preferably consists of, the amino acid sequence of SEQ ID NO: 26. In a preferred embodiment, said first and said second AP205 polypeptide independently comprises (a) an amino acid sequence of a coat protein of RNA bacteriophage AP205, or (b) a mutated amino acid sequence, wherein said mutated amino acid sequence (b) and said amino acid sequence of a coat protein of RNA bacteriophage AP205 (a), have a sequence identity at least 98%, wherein said coat protein of RNA bacteriophage AP205 comprises, preferably consists of, the amino acid sequence of SEQ ID NO: 26.

In a preferred embodiment, said first AP205 polypeptide is fused at its C-terminus directly to the N-terminus of said second AP205 polypeptide.

In another preferred embodiment, said first AP205 polypeptide is fused at its C-terminus via an amino acid spacer to the N-terminus of said second AP205 polypeptide. In another preferred embodiment, said amino acid spacer is selected from the group consisting of:
(a.) a polyglycine linker (Gly)ₙ of a length of n=2-10, preferably a length of n=2-5;
(b.) a glycine-serine linker (GS-linker) comprising at least one glycine and at least one serine, wherein preferably said GS linker has an amino acid sequence of (GS)ᵣ(GₛS_{w})ₜ(GS)ᵤ with r=0 or 1, s=1-5, w=0 or 1; t=1-3 and u=0 or 1; and wherein preferably said glycine-serine linker has a length of at most 15, further preferably of at most 10, amino acids; and
(c.) an amino acid sequence comprising at least one Gly, at least one Ser, and at least one amino acid selected from Thr, Ala, Lys, Asp and Glu, wherein said amino acid sequence has a length of at most 15 amino acids.

In another preferred embodiment, said amino acid spacer is selected from the group consisting of:
(a.) a polyglycine linker (Gly)ₙ of a length of n=2-10, preferably a length of n=2-5; and
(b.) a glycine-serine linker (GS-linker) comprising at least one glycine and at least one serine, wherein preferably said GS linker has an amino acid sequence of (GS)ᵣ(GₛS_{w})ₜ(GS)ᵤ with r=0 or 1, s=1-5, w=0 or 1; t=1-3 and u=0 or 1; and wherein preferably said glycine-serine linker has a length of at most 15, further preferably of at most 10, amino acids.

In another preferred embodiment, said amino acid spacer is selected from the group consisting of:
(a.) a polyglycine linker (Gly)ₙ of a length of n=2-10, preferably a length of n=2-5; and
(b.) a glycine-serine linker (GS-linker) comprising at least one glycine and at least one serine, wherein preferably said GS linker has an amino acid sequence of (GS)ᵣ(GₛS_{w})ₜ(GS)ᵤ with r=0 or 1, s=1-5, w=0 or 1; t=1-3 and u=0 or 1;
   wherein said amino acid spacer has a length of at most 15 amino acids, wherein preferably said amino acid spacer has a length of at most 14, 13, 12, 11, 10, 9, 8, 7, 6, 5, 4, 3, 2 amino acids.

In another preferred embodiment, said amino acid spacer has a length of at most 10 amino acids. In another preferred embodiment, said amino acid spacer has a length of at most 5 amino acids. In another preferred embodiment, said amino acid spacer is a glycine-serine linker (GS-linker) comprising at least one glycine and at least one serine, and has a length of at most 10 amino acids. In another preferred embodiment, said amino acid spacer is a glycine-serine linker (GS-linker) comprising at least one glycine and at least one serine, and has a length of at most 5 amino acids. In another preferred embodiment, said amino acid spacer is the di-amino acid glycine-serine linker GS. In another preferred embodiment, said first AP205 polypeptide is fused at its C-terminus via an amino acid spacer to the N-terminus of said second AP205 polypeptide, wherein said amino acid spacer is a glycine-serine linker, wherein said amino acid spacer has a length of at most 5 amino acids, and wherein preferably said amino acid spacer is the di-amino acid glycine-serine linker GS.

In a preferred embodiment, said amino acid spacer is selected from the group consisting of (a.) a polyglycine linker (Gly)ₙ of a length of n=2-10, preferably a length of n=2-5; and (b.) a glycine-serine linker (GS-linker) comprising at least one glycine and at least one serine, wherein said GS linker has an amino acid sequence of (GS)ᵣ(GₛS_{w})ₜ(GS)ᵤ wherein r, s, t, u are
(i.) r=0, s=1, w=1, t=1, and u=0; (ii.) r=0, s=1, w=1, t=2, and u=0; (iii.) r=0, s=2, w=1, t=1, and u=0; (iv.) r=0, s=3, w=1, t=1, and u=0; (v.) r=0, s=4, w=1, t=1, and u=0; (vi.) r=1, s=1, w=0, t=1, u=0; (vii.) r=1, s=2, w=0, t=1, u=0.

In a preferred embodiment, said amino acid spacer is a glycine-serine linker (GS-linker) comprising at least one glycine and at least one serine, wherein said GS linker has the amino acid sequence of GS, corresponding to the amino acid sequence (GS)ᵣ(GₛS_{w})ₜ(GS)ᵤ wherein r=0, s=1, w=1, t=1, and u=0.

In another preferred embodiment, said amino acid spacer is selected from the group consisting of:
(a.) a polyglycine linker (Gly)ₙ of a length of n=2-10, preferably a length of n=2-5; and
(b.) a glycine-serine linker (GS-linker) comprising at least one glycine and at least one serine, wherein preferably said GS linker has an amino acid sequence of (GS)ᵣ(GₛS_{w})ₜ(GS)ᵤ with r=0 or 1, s=1-5, w=0 or 1; t=1-3 and u=0 or 1;
   wherein said amino acid spacer has a length of at most 15 amino acids.

In a preferred embodiment, said antigenic polypeptide is fused directly to the N-terminus or the C-terminus of said AP205 coat protein dimer. In a preferred embodiment, said antigenic polypeptide is fused directly to the N-terminus of said AP205 coat protein dimer. In a preferred embodiment, said antigenic polypeptide is fused directly to the C-terminus of said AP205 coat protein dimer.

In a preferred embodiment, an antigenic polypeptide is fused directly or via an amino acid linker to the N-terminus of said AP205 coat protein dimer; or an antigenic polypeptide is fused directly or via an amino acid linker to the C-terminus of said AP205 coat protein dimer; or a first antigenic polypeptide is fused directly or via an amino acid linker to the N-terminus of said AP205 coat protein dimer and a second antigenic polypeptide is fused directly or via an amino acid linker to the C-terminus of said AP205 coat protein dimer.

In a preferred embodiment, a first antigenic polypeptide is fused directly or via an amino acid linker to the N-terminus of said AP205 coat protein dimer and a second antigenic polypeptide is fused directly or via an amino acid linker to the C-terminus of said AP205 coat protein dimer. In a preferred embodiment, a first antigenic polypeptide is fused directly to the N-terminus of said AP205 coat protein dimer and a second antigenic polypeptide is fused via an amino acid linker to the C-terminus of said AP205 coat protein dimer. In a preferred embodiment, a first antigenic polypeptide is fused via an amino acid linker to the N-terminus of said AP205 coat protein dimer and a second antigenic polypeptide is fused directly to the C-terminus of said AP205 coat protein dimer. In a preferred embodiment, a first antigenic polypeptide is fused directly to the N-terminus of said AP205 coat protein dimer and a second antigenic polypeptide is fused directly to the C-terminus of said AP205 coat protein dimer. In a preferred embodiment, a first antigenic polypeptide is fused via an amino acid linker to the N-terminus of said AP205 coat protein dimer and a second antigenic polypeptide is fused via an amino acid linker to the C-terminus of said AP205 coat protein dimer.

In a preferred embodiment, said first antigenic polypeptide is fused to the N-terminus of said AP205 coat protein dimer with the opposite orientation as is said second antigenic polypeptide fused to the C-terminus of said AP205 coat protein dimer. By way of example, said first antigenic polypeptide is fused with its C-terminus directly or via an amino acid linker to the N-terminus of said AP205 coat protein dimer, wherein said second antigenic polypeptide is fused with its N-terminus directly or via an amino acid linker to the C-terminus of said AP205 coat protein dimer.

In a preferred embodiment, said first antigenic polypeptide and said second antigenic polypeptide is the same antigenic polypeptide, wherein typically and preferably said first antigenic polypeptide and said second antigenic polypeptide comprises, preferably consists of, the same amino acid sequence. In a preferred embodiment, said first antigenic polypeptide fused directly or via an amino acid linker to the N-terminus of said AP205 coat protein dimer and said second antigenic polypeptide fused directly or via an amino acid linker to the C-terminus of said AP205 coat protein dimer, wherein said first antigenic polypeptide and said second antigenic polypeptide is the same antigenic polypeptide, and typically and preferably comprises, preferably consists of, the same amino acid sequence, and wherein said first antigenic polypeptide is fused directly or via an amino acid linker to the N-terminus of said AP205 coat protein dimer with the opposite orientation as is said second antigenic polypeptide fused directly or via an amino acid linker to the C-terminus of said AP205 coat protein dimer. By way of example, said first antigenic polypeptide is fused with its C-terminus directly or via an amino acid linker to the N-terminus of said AP205 coat protein dimer, wherein said second antigenic polypeptide is fused with its N-terminus directly or via an amino acid linker to the C-terminus of said AP205 coat protein dimer.

In a preferred embodiment, said first antigenic polypeptide and said second antigenic polypeptide are different antigenic polypeptides, and typically and preferably comprise, preferably consist of, different amino acid sequences. In a preferred embodiment, said first antigenic polypeptide fused directly or via an amino acid linker to the N-terminus of said AP205 coat protein dimer and said second antigenic polypeptide fused directly or via an amino acid linker to the C-terminus of said AP205 coat protein dimer, wherein said first antigenic polypeptide and said second antigenic polypeptide are antigenic polypeptides, and typically and preferably comprise, preferably consist of, the different amino acid sequences, wherein said first antigenic polypeptide and said second antigenic polypeptide comprise different epitopes of the same pathogenic or pathologic antigenic polypeptide target.

In a preferred embodiment, a first antigenic polypeptide is fused directly to the N-terminus of said AP205 coat protein dimer and a second antigenic polypeptide is fused directly to the C-terminus of said AP205 coat protein dimer. In a preferred embodiment, said first antigenic polypeptide is fused to the N-terminus of said AP205 coat protein dimer with the opposite orientation as is said second antigenic polypeptide fused to the C-terminus of said AP205 coat protein dimer. By way of example, said first antigenic polypeptide is fused with its C-terminus directly to the N-terminus of said AP205 coat protein dimer, wherein said second antigenic polypeptide is fused with its N-terminus directly to the C-terminus of said AP205 coat protein dimer. In a preferred embodiment, said first antigenic polypeptide and said second antigenic polypeptide is the same antigenic polypeptide, wherein typically and preferably said first antigenic polypeptide and said second antigenic polypeptide comprises, preferably consists of, the same amino acid sequence. In a preferred embodiment, said first antigenic polypeptide fused directly to the N-terminus of said AP205 coat protein dimer and said second antigenic polypeptide fused directly to the C-terminus of said AP205 coat protein dimer, wherein said first antigenic polypeptide and said second antigenic polypeptide is the same antigenic polypeptide, and typically and preferably comprises, preferably consists of, the same amino acid sequence, and wherein said first antigenic polypeptide is fused directly to the N-terminus of said AP205 coat protein dimer with the opposite orientation as is said second antigenic polypeptide fused directly to the C-terminus of said AP205 coat protein dimer. By way of example, said first antigenic polypeptide is fused with its C-terminus directly to the N-terminus of said AP205 coat protein dimer, wherein said second antigenic polypeptide is fused with its N-terminus directly to the C-terminus of said AP205 coat protein dimer.

In a preferred embodiment, said first antigenic polypeptide and said second antigenic polypeptide are different antigenic polypeptides, and typically and preferably comprise, preferably consist of, different amino acid sequences. In a preferred embodiment, said first antigenic polypeptide fused directly to the N-terminus of said AP205 coat protein dimer and said second antigenic polypeptide fused directly to the C-terminus of said AP205 coat protein dimer, wherein said first antigenic polypeptide and said second antigenic polypeptide are antigenic polypeptides, and typically and preferably comprise, preferably consist of, the different amino acid sequences, wherein said first antigenic polypeptide and said second antigenic polypeptide comprise different epitopes of the same pathogenic antigenic polypeptide target.

In another preferred embodiment, said antigenic polypeptide is fused via an amino acid linker to the N-terminus and/or the C-terminus of said AP205 coat protein dimer. In another preferred embodiment, said antigenic polypeptide is fused via an amino acid linker to the N-terminus of said AP205 coat protein dimer. In another preferred embodiment, said antigenic polypeptide is fused via an amino acid linker to the C-terminus of said AP205 coat protein dimer.

In a preferred embodiment, a first antigenic polypeptide is fused via an amino acid linker to the N-terminus of said AP205 coat protein dimer and a second antigenic polypeptide is fused via an amino acid linker to the C-terminus of said AP205 coat protein dimer. In a preferred embodiment, said first antigenic polypeptide is fused to the N-terminus of said AP205 coat protein dimer with the opposite orientation as is said second antigenic polypeptide fused to the C-terminus of said AP205 coat protein dimer. By way of example, said first antigenic polypeptide is fused with its C-terminus via an amino acid linker to the N-terminus of said AP205 coat protein dimer, wherein said second antigenic polypeptide is fused with its N-terminus via an amino acid linker to the C-terminus of said AP205 coat protein dimer. In a preferred embodiment, said first antigenic polypeptide and said second antigenic polypeptide is the same antigenic polypeptide, wherein typically and preferably said first antigenic polypeptide and said second antigenic polypeptide comprises, preferably consists of, the same amino acid sequence. In a preferred embodiment, said first antigenic polypeptide fused via an amino acid linker to the N-terminus of said AP205 coat protein dimer and said second antigenic polypeptide fused via an amino acid linker to the C-terminus of said AP205 coat protein dimer, wherein said first antigenic polypeptide and said second antigenic polypeptide is the same antigenic polypeptide, and typically and preferably comprises, preferably consists of, the same amino acid sequence, and wherein said first antigenic polypeptide is fused via an amino acid spacer to the N-terminus of said AP205 coat protein dimer with the opposite orientation as is said second antigenic polypeptide fused via an amino acid linker to the C-terminus of said AP205 coat protein dimer. By way of example, said first antigenic polypeptide is fused with its C-terminus via an amino acid linker to the N-terminus of said AP205 coat protein dimer, wherein said second antigenic polypeptide is fused with its N-terminus via an amino acid linker to the C-terminus of said AP205 coat protein dimer.

In a preferred embodiment, said first antigenic polypeptide and said second antigenic polypeptide are different antigenic polypeptides, and typically and preferably comprise, preferably consist of, different amino acid sequences. In a preferred embodiment, said first antigenic polypeptide fused via an amino acid linker to the N-terminus of said AP205 coat protein dimer and said second antigenic polypeptide fused via an amino acid linker to the C-terminus of said AP205 coat protein dimer, wherein said first antigenic polypeptide and said second antigenic polypeptide are antigenic polypeptides, and typically and preferably comprise, preferably consist of, the different amino acid sequences, wherein said first antigenic polypeptide and said second antigenic polypeptide comprise different epitopes of the same pathogenic antigenic polypeptide target.

In another preferred embodiment, said amino acid linker is selected from the group consisting of:
(a.) a polyglycine linker (Gly)ₙ of a length of n=2-10, preferably a length of n=2-5; and
(b.) a glycine-serine linker (GS-linker) comprising at least one glycine and at least one serine, wherein preferably said GS linker has an amino acid sequence of (GS)ᵣ(GₛS_{w})ₜ(GS)ᵤ with r=0 or 1, s=1-5, w=0 or 1; t=1-3 and u=0 or 1; and wherein preferably said glycine-serine linker has a length of at most 15, further preferably of at most 10, amino acids;
(c.) an amino acid linker comprising at least one Gly, at least one Ser, and at least one amino acid selected from Thr, Ala, Lys, Asp and Glu, wherein said amino acid sequence has a length of at most 15 amino acids.

In a preferred embodiment, said amino acid linker has a length of at most 10 amino acids. In a preferred embodiment, said amino acid linker has a length of at most 8 amino acids. In a preferred embodiment, said amino acid linker has a length of at most 5 amino acids. In a preferred embodiment, said amino acid linker has a length of 4 amino acids. In a preferred embodiment, said amino acid linker has a length of 3 amino acids. In a preferred embodiment, said amino acid linker has a length of 2 amino acids. In another preferred embodiment, said amino acid linker has a length of 1 amino acid.

In a preferred embodiment, said amino acid linker is selected from the group consisting of (a.) a polyglycine linker (Gly)ₙ of a length of n=2-10, preferably a length of n=2-5; and (b.) a glycine-serine linker (GS-linker) comprising at least one glycine and at least one serine, wherein said GS linker has an amino acid sequence of (GS)ᵣ(GₛS_{w})ₜ(GS)ᵤ with r=0 or 1, s=1-5, w=0 or 1; t=1-3 and u=0 or 1; and wherein preferably said glycine-serine linker has a length of at most 15, further preferably of at most 10, amino acids.

In a preferred embodiment, said amino acid linker is selected from the group consisting of (a.) a polyglycine linker (Gly)ₙ of a length of n=2-10, preferably a length of n=2-5; and (b.) a glycine-serine linker (GS-linker) comprising at least one glycine and at least one serine, wherein said GS linker has has a length of at most 15 amino acids.

In a preferred embodiment, said amino acid linker is selected from the group consisting of (a.) a polyglycine linker (Gly)ₙ of a length of n=2-10, preferably a length of n=2-5; and (b.) a glycine-serine linker (GS-linker) comprising at least one glycine and at least one serine, wherein said GS linker has has a length of at most 10 amino acids. In a preferred embodiment, said amino acid linker is selected from the group consisting of (a.) a polyglycine linker (Gly)ₙ of a length of n=2-5; and (b.) a glycine-serine linker (GS-linker) comprising at least one glycine and at least one serine, wherein said GS linker has has a length of at most 8 amino acids. In a preferred embodiment, said amino acid linker is selected from the group consisting of (a.) a polyglycine linker (Gly)ₙ of a length of n=2-5; and (b.) a glycine-serine linker (GS-linker) comprising at least one glycine and at least one serine, wherein said GS linker has has a length of at most 5 amino acids.

In a preferred embodiment, said amino acid linker is selected from the group consisting of (a.) a polyglycine linker (Gly)ₙ of a length of n=2-10, preferably a length of n=2-5; and (b.) a glycine-serine linker (GS-linker) comprising at least one glycine and at least one serine, wherein said GS linker has an amino acid sequence of (GS)ᵣ(GₛS_{w})ₜ(GS)ᵤ wherein r, s, t, u are
(i.) r=0, s=1, w=1, t=1, and u=0; (ii.) r=0, s=1, w=1, t=2, and u=0; (iii.) r=0, s=2, w=1, t=1, and u=0; (iv.) r=0, s=3, w=1, t=1, and u=0; (v.) r=0, s=4, w=1, t=1, and u=0; (vi.) r=1, s=1, w=0, t=1, u=0; (vii.) r=1, s=2, w=0, t=1, u=0.

In a preferred embodiment, said amino acid linker is a glycine-serine linker (GS-linker) comprising at least one glycine and at least one serine, wherein said GS linker has the amino acid sequence of GS, corresponding to the amino acid sequence (GS)ᵣ(GₛS_{w})ₜ(GS)ᵤ wherein r=0, s=1, w=1, t=1, and u=0.

In a preferred embodiment, said antigenic polypeptide is fused to the N-terminus and/or the C-terminus of said AP205 coat protein dimer either directly or via an amino acid linker, wherein said linker consists of at most five amino acids, preferably of at most four amino acids. In a preferred embodiment, said antigenic polypeptide is fused to the N-terminus and/or the C-terminus of said AP205 coat protein dimer either directly or via an amino acid linker, wherein said linker consists of at most four amino acids, preferably of at most two amino acids. In a preferred embodiment, said antigenic polypeptide is fused to the N-terminus and/or the C-terminus of said AP205 coat protein dimer either directly or via an amino acid linker, wherein said linker consists of two amino acids, wherein preferably said two amino acid linker is the di-amino acid glycine-serine linker GS.

In a preferred embodiment, a first antigenic polypeptide is fused to the N-terminus of said AP205 coat protein dimer either directly or via an amino acid linker and a second antigenic polypeptide is fused to the C-terminus of said AP205 coat protein dimer either directly or via an amino acid linker, wherein said linker consists of at most four amino acids, preferably of at most two amino acids. In a preferred embodiment, a first antigenic polypeptide is fused to the N-terminus of said AP205 coat protein dimer either directly or via an amino acid linker and a second antigenic polypeptide is fused to the C-terminus of said AP205 coat protein dimer either directly or via an amino acid linker, wherein said linker consists of two amino acids, wherein preferably said two amino acid linker is the di-amino acid glycine-serine linker GS. In a preferred embodiment, a first antigenic polypeptide is fused to the N-terminus of said AP205 coat protein dimer directly and a second antigenic polypeptide is fused to the C-terminus of said AP205 coat protein dimer directly.

In a preferred embodiment, said antigenic polypeptide is fused to the N-terminus of said AP205 coat protein dimer either directly or via an amino acid linker, wherein said linker consists of at most five amino acids, preferably of at most four amino acids. In a preferred embodiment, said antigenic polypeptide is fused to the N-terminus of said AP205 coat protein dimer either directly or via an amino acid linker, wherein said linker consists of at most four amino acids, preferably of at most two amino acids. In a preferred embodiment, said antigenic polypeptide is fused to the N-terminus of said AP205 coat protein dimer either directly or via an amino acid linker, wherein said linker consists of two amino acids, wherein preferably said two amino acid linker is the di-amino acid glycine-serine linker GS.

In a preferred embodiment, said antigenic polypeptide is fused to the C-terminus of said AP205 coat protein dimer either directly or via an amino acid linker, wherein said linker consists of at most five amino acids, preferably of at most four amino acids. In a preferred embodiment, said antigenic polypeptide is fused to the C-terminus of said AP205 coat protein dimer either directly or via an amino acid linker, wherein said linker consists of at most four amino acids, preferably of at most two amino acids. In a preferred embodiment, said antigenic polypeptide is fused to the C-terminus of said AP205 coat protein dimer either directly or via an amino acid linker, wherein said linker consists of two amino acids, wherein preferably said two amino acid linker is the di-amino acid glycine-serine linker GS.

In a preferred embodiment, said antigenic polypeptide is fused to the N-terminus of said AP205 coat protein dimer via an amino acid linker, wherein said linker consists of at most five amino acids, preferably of at most four amino acids. In a preferred embodiment, said antigenic polypeptide is fused to the N-terminus of said AP205 coat protein dimer via an amino acid linker, wherein said linker consists of at most four amino acids, preferably of at most two amino acids. In a preferred embodiment, said antigenic polypeptide is fused to the N-terminus of said AP205 coat protein dimer via an amino acid linker, wherein said linker consists of two amino acids, wherein preferably said two amino acid linker is the di-amino acid glycine-serine linker GS.

In a preferred embodiment, said antigenic polypeptide is fused to the C-terminus of said AP205 coat protein dimer via an amino acid linker, wherein said linker consists of at most five amino acids, preferably of at most four amino acids. In a preferred embodiment, said antigenic polypeptide is fused to the C-terminus of said AP205 coat protein dimer via an amino acid linker, wherein said linker consists of at most four amino acids, preferably of at most two amino acids. In a preferred embodiment, said antigenic polypeptide is fused to the C-terminus of said AP205 coat protein dimer via an amino acid linker, wherein said linker consists of two amino acids, wherein preferably said two amino acid linker is the di-amino acid glycine-serine linker GS.

In a preferred embodiment, said AP205 coat protein dimer comprises, preferably consists of, the amino sequence of SEQ ID NO:111 or an amino acid sequence having a sequence identity of at least 90%, preferably of at least 95%, further preferably of at least 98% and again more preferably of at least 99% with said SEQ ID NO:111. In a preferred embodiment, said AP205 coat protein dimer comprises, preferably consists of, the amino sequence of SEQ ID NO:111 or an amino acid sequence having a sequence identity of at least 95% with said SEQ ID NO:111. In a preferred embodiment, said AP205 coat protein dimer comprises, preferably consists of, the amino sequence of SEQ ID NO:111 or an amino acid sequence having a sequence identity of at least 98% with said SEQ ID NO:111. In a preferred embodiment, said AP205 coat protein dimer comprises, preferably consists of, the amino sequence of SEQ ID NO:111 or an amino acid sequence having a sequence identity of at least 99% with said SEQ ID NO:111. In a preferred embodiment, said AP205 coat protein dimer comprises, preferably consists of, the amino sequence of SEQ ID NO:111. In a preferred embodiment, said AP205 coat protein dimer consists of the amino sequence of SEQ ID NO:111 or an amino acid sequence having a sequence identity of at least 95% with said SEQ ID NO:111. In a preferred embodiment, said AP205 coat protein dimer consists of the amino sequence of SEQ ID NO:111 or an amino acid sequence having a sequence identity of at least 98% with said SEQ ID NO:111. In a preferred embodiment, said AP205 coat protein dimer consists of the amino sequence of SEQ ID NO:111 or an amino acid sequence having a sequence identity of at least 99% with said SEQ ID NO:111. In a preferred embodiment, said AP205 coat protein dimer consists of, the amino sequence of SEQ ID NO:111.

In a preferred embodiment, said AP205 coat protein dimer comprises, preferably consists of, the amino sequence of SEQ ID NO: 7 or an amino acid sequence having a sequence identity of at least 90%, preferably of at least 95%, further preferably of at least 98% and again more preferably of at least 99% with said SEQ ID NO: 7. In a preferred embodiment, said AP205 coat protein dimer comprises, preferably consists of, the amino sequence of SEQ ID NO: 7 or an amino acid sequence having a sequence identity of at least 95% with said SEQ ID NO: 7. In a preferred embodiment, said AP205 coat protein dimer comprises, preferably consists of, the amino sequence of SEQ ID NO: 7 or an amino acid sequence having a sequence identity of at least 98% with said SEQ ID NO: 7. In a preferred embodiment, said AP205 coat protein dimer comprises, preferably consists of, the amino sequence of SEQ ID NO: 7 or an amino acid sequence having a sequence identity of at least 99% with said SEQ ID NO: 7. In a preferred embodiment, said AP205 coat protein dimer comprises, preferably consists of, the amino sequence of SEQ ID NO: 7. In a preferred embodiment, said AP205 coat protein dimer consists of the amino sequence of SEQ ID NO: 7 or an amino acid sequence having a sequence identity of at least 95% with said SEQ ID NO: 7. In a preferred embodiment, said AP205 coat protein dimer consists of the amino sequence of SEQ ID NO: 7 or an amino acid sequence having a sequence identity of at least 98% with said SEQ ID NO: 7. In a preferred embodiment, said AP205 coat protein dimer consists of the amino sequence of SEQ ID NO: 7 or an amino acid sequence having a sequence identity of at least 99% with said SEQ ID NO: 7. In a preferred embodiment, said AP205 coat protein dimer consists of the amino sequence of SEQ ID NO: 7.

In a preferred embodiment, said modified AP205 VLP consists of said fusion proteins. Thus, in a preferred embodiment, said modified AP205 VLP consists of about 90, preferably exactly 90, AP205 coat protein dimers to which about 90, preferably exactly 90, antigenic polypeptide are fused to in accordance with the present invention. Thus, the present invention provides a platform with higher antigen density as compared to, for example, mosaic VLP platforms consisting of both native and genetically modified VLP subunits (Pokorski, J K et al, ChemBioChem 2011, 12, 2441-2447; Lino C A et al., J Nanobiotechnol (2017) 15:13; Aves K L et al. Viruses 2020, 12, 185).

In a preferred embodiment, said antigenic polypeptide has a length of at least 50 amino acids. In a preferred embodiment, said antigenic polypeptide has a length of at least 50 amino acids and at most 300 amino acids. In a preferred embodiment, said antigenic polypeptide has a length of at least 60 amino acids. In a preferred embodiment, said antigenic polypeptide has a length of at least 60 amino acids and at most 300 amino acids. In a preferred embodiment, said antigenic polypeptide has a length of at least 70 amino acids. In a preferred embodiment, said antigenic polypeptide has a length of at least 70 amino acids and at most 300 amino acids.

In a preferred embodiment, said antigenic polypeptide has a length of at least 50 amino acids. In a preferred embodiment, said antigenic polypeptide has a length of at least 50 amino acids and at most 250 amino acids. In a preferred embodiment, said antigenic polypeptide has a length of at least 60 amino acids. In a preferred embodiment, said antigenic polypeptide has a length of at least 60 amino acids and at most 250 amino acids. In a preferred embodiment, said antigenic polypeptide has a length of at least 70 amino acids. In a preferred embodiment, said antigenic polypeptide has a length of at least 70 amino acids and at most 250 amino acids.

In a preferred embodiment, said antigenic polypeptide has a length of at least 50 amino acids. In a preferred embodiment, said antigenic polypeptide has a length of at least 50 amino acids and at most 220 amino acids. In a preferred embodiment, said antigenic polypeptide has a length of at least 60 amino acids. In a preferred embodiment, said antigenic polypeptide has a length of at least 60 amino acids and at most 220 amino acids. In a preferred embodiment, said antigenic polypeptide has a length of at least 70 amino acids. In a preferred embodiment, said antigenic polypeptide has a length of at least 70 amino acids and at most 240 amino acids. In a preferred embodiment, said antigenic polypeptide has a length of at least 70 amino acids and at most 230 amino acids. In a preferred embodiment, said antigenic polypeptide has a length of at least 70 amino acids and at most 220 amino acids. In a preferred embodiment, said antigenic polypeptide has a length of at least 70, 75, 80, 85, 90, 95 or 100 amino acids and at most 250 amino acids. In a preferred embodiment, said antigenic polypeptide has a length of at least 70 amino acids and at most 250, 240, 235, 230, 225 or 220 amino acids. In a preferred embodiment, said antigenic polypeptide has a length of at least 80 amino acids. In a preferred embodiment, said antigenic polypeptide has a length of at least 90 amino acids. In a preferred embodiment, said antigenic polypeptide has a length of at least 100 amino acids. In another preferred embodiment, a first antigenic polypeptide is fused directly or via an amino acid linker to the N-terminus of said AP205 coat protein dimer and a second antigenic polypeptide is fused directly or via an amino acid linker to the C-terminus of said AP205 coat protein dimer, wherein said first antigenic polypeptide and said second antigenic polypeptide independently has a length of at least 5, 7, 8, 10 or 12 amino acids. In a preferred embodiment, wherein said first antigenic polypeptide and said second antigenic polypeptide independently has a length of at least 5, 7, 8, 10 or 12 amino acids and at most 250, 200, or 150 amino acids. In a preferred embodiment, wherein said first antigenic polypeptide and said second antigenic polypeptide independently has a length of at least 5, 7, 8, 10 or 12 amino acids and at most 100 amino acids. In a preferred embodiment, wherein said first antigenic polypeptide and said second antigenic polypeptide independently has a length of at least 5, 7, 8, 10 or 12 amino acids and at most 80, 70, 60, 50 amino acids. In a preferred embodiment, wherein said first antigenic polypeptide and said second antigenic polypeptide independently has a length of at least 5 amino acids and at most 80 amino acids. In a preferred embodiment, wherein said first antigenic polypeptide and said second antigenic polypeptide independently has a length of at least 5 amino acids and at most 70 amino acids. In a preferred embodiment, wherein said first antigenic polypeptide and said second antigenic polypeptide independently has a length of at least 5 amino acids and at most 60 amino acids. In a preferred embodiment, wherein said first antigenic polypeptide and said second antigenic polypeptide independently has a length of at least 5 amino acids and at most 50 amino acids. In a preferred embodiment, wherein said first antigenic polypeptide and said second antigenic polypeptide independently has a length of at least 5 amino acids and at most 40 amino acids. In a preferred embodiment, wherein said first antigenic polypeptide and said second antigenic polypeptide independently has a length of at least 5 amino acids and at most 35 amino acids. In a preferred embodiment, wherein said first antigenic polypeptide and said second antigenic polypeptide independently has a length of at least 5 amino acids and at most 30 amino acids. In a preferred embodiment, wherein said first antigenic polypeptide and said second antigenic polypeptide independently has a length of at least 5 amino acids and at most 25 amino acids. In a preferred embodiment, wherein said first antigenic polypeptide and said second antigenic polypeptide independently has a length of at least 5 amino acids and at most 20 amino acids. In a preferred embodiment, wherein said first antigenic polypeptide and said second antigenic polypeptide independently has a length of at least 6 amino acids and at most 80 amino acids. In a preferred embodiment, wherein said first antigenic polypeptide and said second antigenic polypeptide independently has a length of at least 6 amino acids and at most 70 amino acids. In a preferred embodiment, wherein said first antigenic polypeptide and said second antigenic polypeptide independently has a length of at least 6 amino acids and at most 60 amino acids. In a preferred embodiment, wherein said first antigenic polypeptide and said second antigenic polypeptide independently has a length of at least 6 amino acids and at most 50 amino acids. In a preferred embodiment, wherein said first antigenic polypeptide and said second antigenic polypeptide independently has a length of at least 6 amino acids and at most 40 amino acids. In a preferred embodiment, wherein said first antigenic polypeptide and said second antigenic polypeptide independently has a length of at least 6 amino acids and at most 35 amino acids. In a preferred embodiment, wherein said first antigenic polypeptide and said second antigenic polypeptide independently has a length of at least 6 amino acids and at most 30 amino acids. In a preferred embodiment, wherein said first antigenic polypeptide and said second antigenic polypeptide independently has a length of at least 6 amino acids and at most 25 amino acids. In a preferred embodiment, wherein said first antigenic polypeptide and said second antigenic polypeptide independently has a length of at least 6 amino acids and at most 20 amino acids. In a preferred embodiment, wherein said first antigenic polypeptide and said second antigenic polypeptide independently has a length of at least 7 amino acids and at most 80 amino acids. In a preferred embodiment, wherein said first antigenic polypeptide and said second antigenic polypeptide independently has a length of at least 7 amino acids and at most 70 amino acids. In a preferred embodiment, wherein said first antigenic polypeptide and said second antigenic polypeptide independently has a length of at least 7 amino acids and at most 60 amino acids. In a preferred embodiment, wherein said first antigenic polypeptide and said second antigenic polypeptide independently has a length of at least 7 amino acids and at most 50 amino acids. In a preferred embodiment, wherein said first antigenic polypeptide and said second antigenic polypeptide independently has a length of at least 7 amino acids and at most 40 amino acids. In a preferred embodiment, wherein said first antigenic polypeptide and said second antigenic polypeptide independently has a length of at least 7 amino acids and at most 35 amino acids. In a preferred embodiment, wherein said first antigenic polypeptide and said second antigenic polypeptide independently has a length of at least 7 amino acids and at most 30 amino acids. In a preferred embodiment, wherein said first antigenic polypeptide and said second antigenic polypeptide independently has a length of at least 7 amino acids and at most 25 amino acids. In a preferred embodiment, wherein said first antigenic polypeptide and said second antigenic polypeptide independently has a length of at least 7 amino acids and at most 20 amino acids. In a preferred embodiment, wherein said first antigenic polypeptide and said second antigenic polypeptide independently has a length of at least 5, 7, 8, 10 or 12 amino acids and at most 50, 40, 35, 30, 25 or 20 amino acids.

In a preferred embodiment, said antigenic polypeptide is a polypeptide derived from the group consisting of: (a) allergens; (b) viruses; (c) bacteria; (d) parasites; (e) tumors; (f) self-molecules; (g) hormones; (h) growth factors; (i) cytokines; (j) chemokines; and (k) biologically active peptides. In a preferred embodiment, said antigenic polypeptide is of bacterial, viral or mammalian origin. In a preferred embodiment, said antigenic polypeptide is an allergen, a polypeptide derived from a viral pathogen, a polypeptide derived from a bacterial pathogen, a tumor antigen, a self antigen, a polypeptide derived from a hormone, a polypeptide derived from a growth factor, a cytokine or a chemokine. In another preferred embodiment, said antigenic polypeptide is an allergen, a self antigen, a tumor antigen, or a polypeptide of a pathogen. In a preferred embodiment, said antigenic polypeptide is an allergen, a polypeptide derived from a viral pathogen, a polypeptide derived from a bacterial pathogen, a self antigen, a cytokine or a chemokine. In a preferred embodiment, said antigenic polypeptide is an allergen. In a preferred embodiment, said antigenic polypeptide is of viral origin. In a preferred embodiment, said antigenic polypeptide is a polypeptide derived from a virus. In a preferred embodiment, said antigenic polypeptide is of bacterial origin. In a preferred embodiment, said antigenic polypeptide is a polypeptide derived from a bacteria. In a preferred embodiment, said antigenic polypeptide is a polypeptide derived from a parasite. In a preferred embodiment, said antigenic polypeptide is a tumor antigen. In a preferred embodiment, said antigenic polypeptide is a self antigen. In a preferred embodiment, said antigenic polypeptide is a polypeptide derived from a parasite. In a preferred embodiment, said antigenic polypeptide is a hormone. In a preferred embodiment, said antigenic polypeptide is a growth factor. In a preferred embodiment, said antigenic polypeptide is cytokine. In a preferred embodiment, said antigenic polypeptide is chemokine. In a preferred embodiment, said antigenic polypeptide is biologically active peptide.

In a further preferred embodiment said antigenic polypeptide is an allergen, wherein said allergen is derived from the group consisting of: (a) pollen extract; (b) dust extract; (c) dust mite extract; (d) fungal extract; (e) mammalian epidermal extract; (f) feather extract; (g) insect extract; (h) food extract; (i) hair extract; (j) saliva extract; and (k) serum extract. In a further preferred embodiment said antigenic polypeptide is an allergen, wherein said allergen is selected from the group consisting of: (a) trees; (b) grasses; (c) house dust; (d) house dust mite; (e) aspergillus; (f) animal hair; (g) animal feather; (h) bee venom; (i) animal products; (j) plant products; (k) animal dander and (1) peanut allergens.

In a further preferred embodiment said antigenic polypeptide is a recombinant polypeptide derived from an allergen selected from the group consisting of: (a) bee venom phospholipase A2; (b) ragweed pollen Amb a 1; (c) birch pollen Bet v I; (d) white faced hornet venom 5 DoI m V; (e) house dust mite Der p 1; (f) house dust mite Der f 2; (g) house dust mite Der p 2; (h) dust mite Lep d; (i) fungus allergen Alt a 1; (j) fungus allergen Asp f 1; (k) fungus allergen Asp f 16; (1) peanut allergens (m) cat allergen Fel d1; (n) Canine allergens Can f1, Can f2 (o) peanut-derived allergens; or (p) Japanese cedar allergen Cry J2.

In a further preferred embodiment said antigenic polypeptide is a recombinant allergen, wherein said allergen is selected from the group consisting of: (a) bee venom phospholipase A2; (b) ragweed pollen Amb a 1; (c) birch pollen Bet v I; (d) white faced hornet venom 5 DoI m V; (e) house dust mite Der p 1; (f) house dust mite Der f 2; (g) house dust mite Der p 2; (h) dust mite Lep d; (i) fungus allergen Alt a 1; (j) fungus allergen Asp f 1; (k) fungus allergen Asp f 16; (1) peanut allergens (m) cat allergen Fel d1; (n) Canine allergens Can f1, Can f2 (o) peanut-derived allergens; or (p) Japanese cedar allergen Cry J2.

In a further preferred embodiment, said antigenic polypeptide is an allergen derived from Japanese Cedar Cry J 2. Preferably, said antigenic polypeptide is derived from Japanese Cedar Cry J 2 of SEQ ID NO:34. Preferably, said antigenic polypeptide is derived from Japanese Cedar Cry J 2 and comprises the amino acid sequence of SEQ ID NO:34.

In a further preferred embodiment, said antigenic polypeptide is an allergen derived from ragweed pollen Amb a1. Preferably, said antigenic polypeptide is derived from ragweed pollen Amb a 1 of SEQ ID NO:35. Preferably, said antigenic polypeptide is derived from ragweed pollen Amb al and comprises the amino acid sequence of SEQ ID NO:35.

In a further preferred embodiment said antigenic polypeptide is a tumor antigen, wherein said tumor antigen is selected from the group consisting of: (a) a polypeptide of breast cancer cells; (b) a polypeptide of kidney cancer cells; (c) a polypeptide of prostate cancer cells; (d) a polypeptide of skin cancer cells; (e) a polypeptide of brain cancer cells; and (f) a polypeptide of leukemia cells.

In a further preferred embodiment said antigenic polypeptide is a tumor antigen selected from the group consisting of: (a) Her2; (b) ganglioside GD2; (c) EGF-R; (d) carcino embryonic antigen (CEA); (e) CD52; (f) CD21; (g) human melanoma gp100; (h) human melanoma melanA/MART-1; (i) Human melanoma melanA/MART-1 analogue; (j) tyrosinase; (k) NA17-A nt; (1) MAGE3; (m) p53 protein; and (n) antigenic fragments of any of the tumor antigens of (a) to (m).

In a further preferred embodiment said antigenic polypeptide is a polypeptide selected from the group consisting of: (a) IgE, (b) IL-6 (c) receptor activator of nuclear factor kB ligand (RANKL); (d) vascular endothelial growth factor (VEGF); (e) vascular endothelial growth factor receptor (VEGF-R); hepatocyte growth factor (HGF) (f) interleukin-1a; (g) interleukin-1; (h) interleukin-5; (i) interleukin-8; (j) interleukin-13; (k) interleukin-15; (1) interleukin-17 (IL-17); (m) IL-23; (n) Ghrelin; (o) angiotensin; (p) chemokine (C-C motif) (CCL21); (q) chemokine (C-X motif) (CXCL 12); (r) stromal cell derived factor 1 (SDF-I); (s) macrophage colony stimulating factor (M-CSF); (t) monocyte chemotactic protein 1 (MCP-I); (u) endoglin; (v) resistin; (w) gonadotropin releasing hormone (GnRH); (x) growth hormone releasing (GHRH); (y) lutenizing hormone releasing hormone (LHRH); (z) thyreotropin releasing hormone (TRH); (aa) macrophage migration inhibitory factor (MIF); (bb) glucose-dependent insulinotropic peptide (GIP); (cc) eotaxin; (dd) bradykinin; (ee) Des-Arg bradykinin; (ff) B-lymphocyte chemoattractant (BLC); (gg) macrophage colony stimulating factor M-CSF; (hh) tumor necrosis factor α (TNFα); (ii) amyloid beta peptide (Aβ1-42); (jj) amyloid beta peptide (AP3-6); (kk) human IgE; (ii) CCR5 extracellular domain; (mm) CXCR4 extracellular domain; (nn) Gastrin; (oo) CETP; (pp) C5a; (qq) epidermal growth factor receptor (EGF-R); (rr) CGRP; (ss) α-synuclein; (tt) calcitonin gene-related peptide (CGRP) (uu) Amylin; (vv) myostatin; (ww) interleukin-4; (xx) thymic stromal lymphopoietin; (yy) interleukin-33; (zz) interleukin-25; (aaa) interleukin-13 or (bbb) a fragment of any one of the polypeptides (a) to (aaa); and (ccc) an antigenic mutant or fragment of any one of the polypeptides (a) to (aaa).

In a further preferred embodiment said antigenic polypeptide is a self antigen, wherein said self antigen is a polypeptide selected from the group consisting of: (a) IgE, (b) IL-6 (c) receptor activator of nuclear factor kB ligand (RANKL); (d) vascular endothelial growth factor (VEGF); (e) vascular endothelial growth factor receptor (VEGF-R); hepatocyte growth factor (HGF) (f) interleukin-1 α; (g) interleukin-1 β; (h) interleukin-5; (i) interleukin-8; (j) inter leukin-13; (k) interleukin-15; (1) interleukin-17 (IL-17); (m) IL-23; (n) Ghrelin; (o) angiotensin; (p) chemokine (C-C motif) (CCL21); (q) chemokine (C-X motif) (CXCL 12); (r) stromal cell derived factor 1 (SDF-I); (s) macrophage colony stimulating factor (M-CSF); (t) monocyte chemotactic protein 1 (MCP-I); (u) endoglin; (v) resistin; (w) gonadotropin releasing hormon (GnRH); (x) growth hormon releasing (GHRH); (y) lutenizing hormon releasing hormon (LHRH); (z) thyreotropin releasing hormon (TRH); (aa) macrophage migration inhibitory factor (MIF); (bb) glucose-dependent insulinotropic peptide (GIP); (cc) eotaxin; (dd) bradykinin; (ee) Des-Arg bradykinin; (ff) B-lymphocyte chemoattractant (BLC); (gg) macrophage colony stimulating factor M-CSF; (hh) tumor necrosis factor α (TNFα); (ii) amyloid beta peptide (Aβ1-42); (jj) amyloid beta peptide (Aβ-6); (kk) human IgE; (ii) CCR5 extracellular domain; (mm) CXCR4 extracellular domain; (nn) Gastrin; (oo) CETP; (pp) C5a; (qq) epidermal growth factor receptor (EGF-R); (rr) CGRP; (ss) α-synuclein; (tt) calcitonin gene-related peptide (CGRP) (uu) Amylin; (vv) myostatin; (ww) interleukin-4; (xx) thymic stromal lymphopoietin; (yy) interleukin-33; (zz) interleukin-25; (aaa) interleukin-13 or (bbb) a fragment of any one of the polypeptides (a) to (aaa); and (ccc) an antigenic mutant or fragment of any one of the polypeptides (a) to (aaa).

In a preferred embodiment, said antigenic polypeptide is interleukin 17 (IL-17), preferably human IL-17. Interleukin 17 is a T cell-derived cytokine that induces the release of pro-inflammatory mediators in a wide range of cell types. Aberrant Th17 responses and overexpression of IL-17 have been implicated in a number of autoimmune disorders including rheumatoid arthritis psoriasis, ankylosing spondylitis, and multiple sclerosis. Molecules blocking IL-17 such as IL-17-specific monoclonal antibodies have proved to be effective in ameliorating disease in animal models. Moreover, active immunization targeting IL-17 has recently been suggested using virus-like particles conjugated with recombinant IL-17 (Rohn T A, et al., Eur J Immunol (2006) 36: 1-11). Immunization with IL-17-VLP induced high levels of anti-IL-17 antibodies thereby overcoming natural tolerance, even in the absence of added adjuvant. Mice immunized with IL-17-VLP had lower incidence of disease, slower progression to disease and reduced scores of disease severity in both collagen-induced arthritis and experimental autoimmune encephalomyelitis.

Thus, in a preferred embodiment, said antigenic polypeptide comprises, or preferably consists of SEQ ID NO:36. Furthermore, the inventive modified AP205 VLPs are used in a method of treating an inflammatory disease, preferably a chronic inflammatory disease in an animal or human. Preferably, said inflammatory disease is selected from RA, MS, Psoriasis, asthma, Crohns, Colitis, COPD, diabetes, neurodermatitis (allergic dermatitis), again preferably wherein said inflammatory disease is MS, and wherein further preferably said antigenic polypeptide comprises, or preferably consists of SEQ ID NO:82.

In another preferred embodiment, said antigenic polypeptide is IL-5, preferably human, canine, feline or horse IL-5. In another preferred embodiment, said antigenic polypeptide is human IL-5. In again a further preferred embodiment, said antigenic polypeptide comprises, or preferably consists of SEQ ID NO:37. Furthermore, the inventive modified AP205 VLPs comprising antigenic polypeptides of IL-5 are used in a method of treating an inflammatory disease, preferably a chronic inflammatory disease in an animal or human. Preferably, said inflammatory disease is selected from RA, MS, Psoriasis, asthma, Crohns, Colitis, COPD, diabetes, neurodermatitis (allergic dermatitis), eosinophilic granulomatosis, feline atopic skin syndrome and insect bite hypersensitivity. In another preferred embodiment, said antigenic polypeptide comprises, or preferably consists of SEQ ID NO:37.

In another preferred embodiment, said antigenic polypeptide is canine IL-5. In again a further preferred embodiment, said antigenic polypeptide comprises, or preferably consists of SEQ ID NO:38 or an amino acid sequence having a sequence identity of at least 90%, preferably of at least 95%, with SEQ ID NO:38. Preferably, said antigenic polypeptide comprises, or preferably consists of SEQ ID NO:38. In another preferred embodiment, said antigenic polypeptide consists of SEQ ID NO:38.

In another preferred embodiment, said antigenic polypeptide is feline IL-5. In a preferred embodiment, said antigenic polypeptide comprises, or preferably consists of, SEQ ID NO:39, SEQ ID NO:40, SEQ ID:41 or an amino acid sequence having a sequence identity of at least 90%, preferably of at least 92%, further preferably of at least 95%, and again further preferably of at least 98% amino acid sequence identity with SEQ ID NO:39, SEQ ID NO:40, SEQ ID:41. In a further preferred embodiment, said antigenic polypeptide comprises SEQ ID NO:39, SEQ ID NO:40 or SEQ ID:41. In a further preferred embodiment, said antigenic polypeptide consists of SEQ ID NO:39, SEQ ID NO:40 or SEQ ID:41. In a further preferred embodiment, said antigenic polypeptide comprises, or preferably consists of, SEQ ID NO:39 or an amino acid sequence having a sequence identity of at least 90%, preferably of at least 92%, further preferably of at least 95%, and again further preferably of at least 98% amino acid sequence identity with SEQ ID NO:39.

In another preferred embodiment, said antigenic polypeptide is equine IL-5. In a preferred embodiment, said antigenic polypeptide comprises, or preferably consists of, SEQ ID NO:42 or an amino acid sequence having a sequence identity of at least 90%, preferably of at least 92%, further preferably of at least 95%, and again further preferably of at least 98% amino acid sequence identity with SEQ ID NO:42.

In another preferred embodiment, said antigenic polypeptide is IL-4, preferably human Il-4. In again a further preferred embodiment, said antigenic polypeptide comprises, or preferably consists of SEQ ID NO:43. In another preferred embodiment, said antigenic polypeptide consists of SEQ ID NO:43.

In another preferred embodiment, said antigenic polypeptide is canine IL-4. In again a further preferred embodiment, said antigenic polypeptide comprises, or preferably consists of SEQ ID NO:44 or an amino acid sequence having a sequence identity of at least 90%, preferably of at least 95%, with SEQ ID NO:44. Preferably, said antigenic polypeptide comprises, or preferably consists of SEQ ID NO:44. In another preferred embodiment, said antigenic polypeptide consists of SEQ ID NO:44.

In another preferred embodiment, said antigenic polypeptide is feline IL-4. In again a further preferred embodiment, said antigenic polypeptide comprises, or preferably consists of SEQ ID NO:45 or an amino acid sequence having a sequence identity of at least 90%, preferably of at least 95%, with SEQ ID NO:45. Preferably, said antigenic polypeptide comprises, or preferably consists of SEQ ID NO:45. Preferably, said antigenic polypeptide comprises, or preferably consists of SEQ ID NO:45. In another preferred embodiment, said antigenic polypeptide consists of SEQ ID NO:45.

In another preferred embodiment, said antigenic polypeptide is equine IL-4.

In another preferred embodiment, said antigenic polypeptide is IL-13, preferably human IL-13. Furthermore, the inventive modified AP205 VLPs comprising antigenic polypeptides of IL-13 are used in a method of treating an inflammatory disease, preferably an allergic inflammation, allergic lung disease, asthma or atopic dermatitis. In again a further preferred embodiment, said antigenic polypeptide comprises, or preferably consists of SEQ ID NO:46. In another preferred embodiment, said antigenic polypeptide consists of SEQ ID NO:46.

In another preferred embodiment, said antigenic polypeptide is canine IL-13. In again a further preferred embodiment, said antigenic polypeptide comprises, or preferably consists of SEQ ID NO:47 or an amino acid sequence having a sequence identity of at least 90%, preferably of at least 95%, with SEQ ID NO:47. Preferably, said antigenic polypeptide comprises, or preferably consists of SEQ ID NO:47. In another preferred embodiment, said antigenic polypeptide consists of SEQ ID NO:47.

In another preferred embodiment, said antigenic polypeptide is feline IL-13. In again a further preferred embodiment, said antigenic polypeptide comprises, or preferably consists of SEQ ID NO:48 or an amino acid sequence having a sequence identity of at least 90%, preferably of at least 95%, with SEQ ID NO:48. Preferably, said antigenic polypeptide comprises, or preferably consists of SEQ ID NO:48. In another preferred embodiment, said antigenic polypeptide consists of SEQ ID NO:48.

In another preferred embodiment, said antigenic polypeptide is equine IL-13. In again a further preferred embodiment, said antigenic polypeptide comprises, or preferably consists of SEQ ID NO:49 or an amino acid sequence having a sequence identity of at least 90%, preferably of at least 95%, with SEQ ID NO:49. Preferably, said antigenic polypeptide comprises, or preferably consists of SEQ ID NO:49. In another preferred embodiment, said antigenic polypeptide consists of SEQ ID NO:49.

In a further preferred embodiment, said antigenic polypeptide is TNFα. Furthermore, the inventive modified AP205 VLPs comprising antigenic polypeptides of TNFα are used in a method of treating an inflammatory disease, preferably multisystem inflammatory diseases, rheumatoid arthritis, inflammatory bowel disease, Crohn's disease, ulcerative colitis, psoriasis, psoriatic arthritis, juvenile idiopathic arthritis or ankylosing spondylitis. In another preferred embodiment, said antigenic polypeptide is IL-1α, preferably human IL-1α. In again a further preferred embodiment, said antigenic polypeptide comprises, or preferably consists of SEQ ID NO:50. In another preferred embodiment, said antigenic polypeptide consists of SEQ ID NO:50.

In another preferred embodiment, said antigenic polypeptide is canine IL-1a. In again a further preferred embodiment, said antigenic polypeptide comprises, or preferably consists of SEQ ID NO:51 or an amino acid sequence having a sequence identity of at least 90%, preferably of at least 95%, with SEQ ID NO:51. Preferably, said antigenic polypeptide comprises, or preferably consists of SEQ ID NO:51. In another preferred embodiment, said antigenic polypeptide consists of SEQ ID NO:51.

In another preferred embodiment, said antigenic polypeptide is feline IL-1a. In again a further preferred embodiment, said antigenic polypeptide comprises, or preferably consists of SEQ ID NO:52 or an amino acid sequence having a sequence identity of at least 90%, preferably of at least 95%, with SEQ ID NO:52. Preferably, said antigenic polypeptide comprises, or preferably consists of SEQ ID NO:52. In another preferred embodiment, said antigenic polypeptide consists of SEQ ID NO:52.

In another preferred embodiment, said antigenic polypeptide is equine IL-1a. In again a further preferred embodiment, said antigenic polypeptide comprises, or preferably consists of SEQ ID NO:53 or an amino acid sequence having a sequence identity of at least 90%, preferably of at least 95%, with SEQ ID NO:53. Preferably, said antigenic polypeptide comprises, or preferably consists of SEQ ID NO:53. In another preferred embodiment, said antigenic polypeptide consists of SEQ ID NO:53.

In another very preferred embodiment, said antigenic polypeptide is IL-33, preferably human IL-33. Furthermore, the inventive modified AP205 VLPs comprising antigenic polypeptides of IL-33 are used in a method of treating an inflammatory disease, preferably atopic dermatitis, asthma, a cardiovascular disease, a musculoskeletal disease, inflammatory bowel disease, or an allergy such as food allergy, or cancer or Alzheimer disease. In again a further very preferred embodiment, said antigenic polypeptide comprises, or preferably consists of SEQ ID NO:54. In another preferred embodiment, said antigenic polypeptide consists of SEQ ID NO:54.

Canine atopic dermatitis is a form of inflammation of skin, causing itch, which promotes dogs to scratch extensively and eventually, loose their fur around scratched places. Several interleukins appear to be involved in the driving of itching, including canine interleukins cIL-31 and cIL-33. It has been shown previously that vaccination of dogs with the inventive modified VLPs, decorated with cIL-31 raises autoantibodies and reduces itching. Similar effects are plausible when the inventive modified VLPs decorated with cIL-33 are used as evidenced by the data of Example 3.

Thus, in another very preferred embodiment, said antigenic polypeptide is canine IL-33. In again a further very preferred embodiment, said antigenic polypeptide comprises, or preferably consists of any one of SEQ ID NO:55, SEQ ID NO:56, SEQ ID NO:112 or SEQ ID NO:113, or an amino acid sequence having a sequence identity of at least 90%, preferably of at least 95%, with any one of SEQ ID NO:55, SEQ ID NO:56, SEQ ID NO:112 or SEQ ID NO:113. Preferably, said antigenic polypeptide comprises, or preferably consists of any one of SEQ ID NO:55, SEQ ID NO:56, SEQ ID NO:112 or SEQ ID NO:113. In another preferred embodiment, said antigenic polypeptide consists any one of SEQ ID NO:55, SEQ ID NO:56, SEQ ID NO:112 or SEQ ID NO:113. In again a further very preferred embodiment, said antigenic polypeptide comprises, or preferably consists of SEQ ID NO:56 or an amino acid sequence having a sequence identity of at least 90%, preferably of at least 95%, with SEQ ID NO:56. Preferably, said antigenic polypeptide comprises, or preferably consists of SEQ ID NO:56. In another preferred embodiment, said antigenic polypeptide consists of SEQ ID NO:56. In again a further very preferred embodiment, said antigenic polypeptide comprises, or preferably consists of SEQ ID NO:113 or an amino acid sequence having a sequence identity of at least 90%, preferably of at least 95%, with SEQ ID NO: 113. Preferably, said antigenic polypeptide comprises, or preferably consists of SEQ ID NO: 113. In another preferred embodiment, said antigenic polypeptide consists of SEQ ID NO: 113.

In another very preferred embodiment, said antigenic polypeptide is feline IL-33. In again a further very preferred embodiment, said antigenic polypeptide comprises, or preferably consists of SEQ ID NO:57 or an amino acid sequence having a sequence identity of at least 90%, preferably of at least 95%, with SEQ ID NO:57. Preferably, said antigenic polypeptide comprises, or preferably consists of SEQ ID NO:57. In another preferred embodiment, said antigenic polypeptide consists of SEQ ID NO:57.

In another very preferred embodiment, said antigenic polypeptide is equine IL-33. In again a further very preferred embodiment, said antigenic polypeptide comprises, or preferably consists of SEQ ID NO:58 or an amino acid sequence having a sequence identity of at least 90%, preferably of at least 95%, with SEQ ID NO:58. Preferably, said antigenic polypeptide comprises, or preferably consists of SEQ ID NO:58. In another preferred embodiment, said antigenic polypeptide consists of SEQ ID NO:58.

In another preferred embodiment, said antigenic polypeptide is IL-25, preferably human IL-25. In again a further preferred embodiment, said antigenic polypeptide comprises, or preferably consists of SEQ ID NO:59. In another preferred embodiment, said antigenic polypeptide consists of SEQ ID NO:59.

In another preferred embodiment, said antigenic polypeptide is canine IL-25. In again a further preferred embodiment, said antigenic polypeptide comprises, or preferably consists of SEQ ID NO:60 or an amino acid sequence having a sequence identity of at least 90%, preferably of at least 95%, with SEQ ID NO:60. Preferably, said antigenic polypeptide comprises, or preferably consists of SEQ ID NO:60. In another preferred embodiment, said antigenic polypeptide consists of SEQ ID NO:60.

In another preferred embodiment, said antigenic polypeptide is feline IL-25. In again a further preferred embodiment, said antigenic polypeptide comprises, or preferably consists of SEQ ID NO:61 or an amino acid sequence having a sequence identity of at least 90%, preferably of at least 95%, with SEQ ID NO:61. Preferably, said antigenic polypeptide comprises, or preferably consists of SEQ ID NO:61. In another preferred embodiment, said antigenic polypeptide consists of SEQ ID NO:61.

In another preferred embodiment, said antigenic polypeptide is equine IL-25. In again a further preferred embodiment, said antigenic polypeptide comprises, or preferably consists of SEQ ID NO:62 or an amino acid sequence having a sequence identity of at least 90%, preferably of at least 95%, with SEQ ID NO:62. Preferably, said antigenic polypeptide comprises, or preferably consists of SEQ ID NO:62. In another preferred embodiment, said antigenic polypeptide consists of SEQ ID NO:62.

In a further preferred embodiment, said antigenic polypeptide is IL-1β, preferably human IL-1β. Furthermore, the inventive modified AP205 VLPs comprising antigenic polypeptides of IL-1β are used in a method of treating an inflammatory disease, preferably multisystem inflammatory diseases associated with inflammasome dysregulation including osteoarthritis, juvenile idiopathic arthritis, Familial Mediterranean Fever, cryopyrin associated periodic syndrome, Muckle-Wells Syndrome, hyperimmunoglobulin D syndrome, Stills disease, gouty arthritis, rheumatoid arthritis, chronic obstructive pulmonary disease and coronary artery disease. In again a further preferred embodiment, said antigenic polypeptide comprises, or preferably consists of SEQ ID NO:63.

In a further preferred embodiment, said antigenic polypeptide is canine IL-1β. In a preferred embodiment, said antigenic polypeptide comprises, or preferably consists of, SEQ ID NO:64, SEQ ID NO:65, SEQ ID NO:66 or an amino acid sequence having a sequence identity of at least 90%, preferably of at least 92%, further preferably of at least 95%, and again further preferably of at least 98% amino acid sequence identity with SEQ ID NO:64, SEQ ID NO:65, SEQ ID NO:66. In a further preferred embodiment, said antigenic polypeptide comprises SEQ ID NO:64, SEQ ID NO:65, SEQ ID NO:66. In a further preferred embodiment, said antigenic polypeptide consists of SEQ ID NO:64, SEQ ID NO:65, SEQ ID NO:66. In a further preferred embodiment, said antigenic polypeptide comprises, or preferably consists of, SEQ ID NO:64 or an amino acid sequence having a sequence identity of at least 90%, preferably of at least 92%, further preferably of at least 95%, and again further preferably of at least 98% amino acid sequence identity with SEQ ID NO:64.

In a further preferred embodiment, said antigenic polypeptide is feline IL-1β. In again a further preferred embodiment, said antigenic polypeptide comprises, or preferably consists of SEQ ID NO:67.

In a further preferred embodiment, said antigenic polypeptide is IL-12/23, preferably human IL-12/23. In a further preferred embodiment, said antigenic polypeptide is canine IL-12/23. In a further preferred embodiment, said antigenic polypeptide is feline IL-12/23. In a further preferred embodiment, said antigenic polypeptide is equine IL-12/23.

In another preferred embodiment, said antigenic polypeptide is IL-31, preferably human IL-31. Furthermore, the inventive modified AP205 VLPs comprising antigenic polypeptides of IL-31 are used in a method of treating an inflammatory disease, preferably atopic dermatitis, bullous pemphigoid, chronic urticaria or asthma. In again a further preferred embodiment, said antigenic polypeptide comprises, or preferably consists of SEQ ID NO:68. In another preferred embodiment, said antigenic polypeptide consists of SEQ ID NO:68.

In another preferred embodiment, said antigenic polypeptide is canine IL-31. In again a further preferred embodiment, said antigenic polypeptide comprises, or preferably consists of SEQ ID NO:69 or an amino acid sequence having a sequence identity of at least 90%, preferably of at least 95%, with SEQ ID NO:69. Preferably, said antigenic polypeptide comprises, or preferably consists of SEQ ID NO:69. In another preferred embodiment, said antigenic polypeptide consists of SEQ ID NO:69.

In another preferred embodiment, said antigenic polypeptide is feline IL-31. In again a further preferred embodiment, said antigenic polypeptide comprises, or preferably consists of SEQ ID NO:70 or an amino acid sequence having a sequence identity of at least 90%, preferably of at least 95%, with SEQ ID NO:70. Preferably, said antigenic polypeptide comprises, or preferably consists of SEQ ID NO:70. In another preferred embodiment, said antigenic polypeptide consists of SEQ ID NO:70.

In another preferred embodiment, said antigenic polypeptide is equine IL-31. In again a further preferred embodiment, said antigenic polypeptide comprises, or preferably consists of SEQ ID NO:71 or an amino acid sequence having a sequence identity of at least 90%, preferably of at least 95%, with SEQ ID NO:71. Preferably, said antigenic polypeptide comprises, or preferably consists of SEQ ID NO:71. In another preferred embodiment, said antigenic polypeptide consists of SEQ ID NO:71.

In another preferred embodiment, said antigenic polypeptide is thymic stromal lymphopoietin (TLSP), preferably human thymic stromal lymphopoietin (TLSP). In again a further preferred embodiment, said antigenic polypeptide comprises, or preferably consists of SEQ ID NO:72. In another preferred embodiment, said antigenic polypeptide consists of SEQ ID NO:72.

In another preferred embodiment, said antigenic polypeptide is canine TLSP. In again a further preferred embodiment, said antigenic polypeptide comprises, or preferably consists of SEQ ID NO:73 or an amino acid sequence having a sequence identity of at least 90%, preferably of at least 95%, with SEQ ID NO:73. Preferably, said antigenic polypeptide comprises, or preferably consists of SEQ ID NO:73. In another preferred embodiment, said antigenic polypeptide consists of SEQ ID NO:73.

In another preferred embodiment, said antigenic polypeptide is feline TLSP. In again a further preferred embodiment, said antigenic polypeptide comprises, or preferably consists of SEQ ID NO:74 or an amino acid sequence having a sequence identity of at least 90%, preferably of at least 95%, with SEQ ID NO:74. Preferably, said antigenic polypeptide comprises, or preferably consists of SEQ ID NO:74. In another preferred embodiment, said antigenic polypeptide consists of SEQ ID NO:74.

In another preferred embodiment, said antigenic polypeptide is equine TLSP. In again a further preferred embodiment, said antigenic polypeptide comprises, or preferably consists of SEQ ID NO:75 or an amino acid sequence having a sequence identity of at least 90%, preferably of at least 95%, with SEQ ID NO:75. Preferably, said antigenic polypeptide comprises, or preferably consists of SEQ ID NO:75. In another preferred embodiment, said antigenic polypeptide consists of SEQ ID NO:75.

In again a further preferred embodiment, said antigenic polypeptide is IgE or a peptide or domain comprised in IgE.

In again a further preferred embodiment, said antigenic polypeptide is a peptide derived the N-terminus from Aβ-1-42 (SEQ ID NO:76), in particular a fragment of Aβ-1-42 (SEQ ID NO: 76) of at most 7 consecutive amino acids in length, preferably a fragment of Aβ-1-42 (SEQ ID NO: 76) of at most 6 consecutive amino acids in length. Thus, in a further preferred embodiment, said antigenic polypeptide is selected from A3-1-6 (SEQ ID NO:77), A3-1-7 (SEQ ID NO:78), A3-3-6 (SEQ ID NO:79), A3-1-5 (SEQ ID NO:80), Aβ-2-6 (SEQ ID NO:81), or Aβ-3-7 (SEQ ID NO:82).

In another preferred embodiment, said antigenic polypeptide is α-synuclein or a peptide derived from α-synuclein, and wherein preferably said peptide consists of 6 to 14 amino acids, and wherein further preferably said antigenic polypeptide is a peptide derived from α-synuclein selected from any one of SEQ D NO:83, SEQ ID NO:84, SEQ ID NO:85 and SEQ ID NO:86. Further preferred peptides derived from α-synuclein are disclosed in WO 2011/020133, which is incorporated herein by way of reference.

Alpha-synuclein (α-Syn), a small protein with multiple physiological and pathological functions, is one of the dominant proteins found in Lewy Bodies, a pathological hallmark of Lewy body disorders, including Parkinson's disease (PD). More recently, α-Syn has been found in body fluids, including blood and cerebrospinal fluid, and is likely produced by both peripheral tissues and the central nervous system. Exchange of α-Syn between the brain and peripheral tissues could have important pathophysiologic and therapeutic implications (Gardai S J et al., PLoS ONE (2013) 8(8): e71634). The evidence implicating alpha-synuclein (α-syn) in the pathogenesis of Parkinson's Disease (PD) is overwhelming.

Thus, in a further preferred embodiment, said antigenic polypeptide is selected from any one of the sequences selected from SEQ D NO:83, SEQ ID NO:84, SEQ ID NO:85 and SEQ ID NO:86. In a further preferred embodiment, said antigenic polypeptide is SEQ D NO:83. In a further preferred embodiment, said antigenic polypeptide is SEQ D NO:84. In a further preferred embodiment, said antigenic polypeptide is SEQ D NO:85. In a further preferred embodiment, said antigenic polypeptide is SEQ D NO:86.

In another preferred embodiment, a first antigenic polypeptide is fused directly or via an amino acid linker to the N-terminus of said AP205 coat protein dimer and a second antigenic polypeptide is fused directly or via an amino acid linker to the C-terminus of said AP205 coat protein dimer, wherein first antigenic polypeptide and said second antigenic polypeptide is independently selected from any one of the SEQ ID NO:77 to SEQ ID NO:86.

In again a further preferred embodiment, said antigenic polypeptide is Amylin.

In a further preferred embodiment, said antigenic polypeptide is derived from African Swine Fever (ASF) protein. In a preferred embodiment, said antigenic polypeptide comprises, preferably is, SEQ ID NO:102. In another preferred embodiment, a first antigenic polypeptide is fused directly or via an amino acid linker to the N-terminus of said AP205 coat protein dimer and a second antigenic polypeptide is fused directly or via an amino acid linker to the C-terminus of said AP205 coat protein dimer, wherein first antigenic polypeptide and said second antigenic polypeptide is derived from African Swine Fever (ASF) protein. In another preferred embodiment, a first antigenic polypeptide is fused directly or via an amino acid linker to the N-terminus of said AP205 coat protein dimer and a second antigenic polypeptide is fused directly or via an amino acid linker to the C-terminus of said AP205 coat protein dimer, wherein first antigenic polypeptide and said second antigenic polypeptide is SEQ ID NO:102. This modified AP205 VLP comprising antigenic polypeptides derived from African Swine Fever (ASF) protein can be used to address African Swine Fever infections.

In a further preferred embodiment, said antigenic polypeptide is Gonadotropin Releasing Hormone (GnRH). In one preferred embodiment, the antigenic polypeptide is GnRH or a fragment thereof. Such fragments useful in the production of modified AP205 VLPs and vaccines in accordance with the present invention are disclosed in WO2006/027300, which is incorporated herein by reference in its entirety. In a preferred embodiment, said antigenic polypeptide comprises, preferably is, SEQ ID NO: 114 or SEQ ID NO:115. In another preferred embodiment, a first antigenic polypeptide is fused directly or via an amino acid linker to the N-terminus of said AP205 coat protein dimer and a second antigenic polypeptide is fused directly or via an amino acid linker to the C-terminus of said AP205 coat protein dimer, wherein first antigenic polypeptide and said second antigenic polypeptide is GnRH or a fragment thereof. In another preferred embodiment, a first antigenic polypeptide is fused directly or via an amino acid linker to the N-terminus of said AP205 coat protein dimer and a second antigenic polypeptide is fused directly or via an amino acid linker to the C-terminus of said AP205 coat protein dimer, wherein first antigenic polypeptide and said second antigenic polypeptide is independently selected from SEQ ID NO:114 or SEQ ID NO:115. In a further preferred embodiment, the N-terminal glutamic acid of SEQ ID NO:114 is a pyroglutamic acid (pGlu or pE).

This modified AP205 VLP comprising antigenic polypeptides derived from GnRH can be used to address boar taint, fertility and behavior management. Thus, this modified AP205 VLP comprising antigenic polypeptides derived from GnRH can be administered to a mammal, such as pig to prevent the boar taint in the meat. This modified AP205 VLP comprising GnRH can be administered to an animal, such as dog, cat, sheep, cattle, horse to control their behaviour and/or to reduce their reproductivity. This modified 205 VLP comprising GnRH can be administered to human having gonadal steroid hormone dependent cancers. Moreover, this modified 205 VLP comprising GnRH can be administered to an animal or human to lower steroid hormone, preferably testosterone, levels in an animal or human.

In a preferred embodiment, said antigenic polypeptide is angiotensin I or a peptide derived from angiotensin I. In another preferred embodiment, said antigenic polypeptide is angiotensin II or a peptide derived from angiotensin II.

Modified AP205 VLP comprising angiotensin derived antigenic polypeptides are useful for the treatment of diseases or disorders associated with the renin-activated angiotensin system, and in particular for the treatment of diseases selected from the group consisting of hypertension and high blood pressure, stroke, infarction, congestive heart failure, kidney failure, preferably cat chronic kidney disease, and retinal hemorrhage. Such angiotensin derived antigenic polypeptides are disclosed in WO03031466, which is incorporated herein by reference in its entirety. In a preferred embodiment, said antigenic polypeptide comprises, preferably is, SEQ ID NO:116, SEQ ID NO:117 or SEQ ID NO:118.

In another preferred embodiment, a first antigenic polypeptide is fused directly or via an amino acid linker to the N-terminus of said AP205 coat protein dimer and a second antigenic polypeptide is fused directly or via an amino acid linker to the C-terminus of said AP205 coat protein dimer, wherein first antigenic polypeptide and said second antigenic polypeptide is an angiotensin derived antigenic polypeptide. In another preferred embodiment, a first antigenic polypeptide is fused directly or via an amino acid linker to the N-terminus of said AP205 coat protein dimer and a second antigenic polypeptide is fused directly or via an amino acid linker to the C-terminus of said AP205 coat protein dimer, wherein first antigenic polypeptide and said second antigenic polypeptide is independently selected from SEQ ID NO:116, SEQ ID NO:117 or SEQ ID NO:118.

In a further preferred embodiment, said antigenic polypeptide is eotaxin.

In another preferred embodiment, said antigenic polypeptide is myostatin, preferably cow myostatin. In again a further preferred embodiment, said antigenic polypeptide comprises, or preferably consists of SEQ ID NO:87 or an amino acid sequence having a sequence identity of at least 90%, preferably of at least 95%, with SEQ ID NO:87. Preferably, said antigenic polypeptide comprises, or preferably consists of SEQ ID NO:87. In another preferred embodiment, said antigenic polypeptide consists of SEQ ID NO:87.

In a further preferred embodiment said antigenic polypeptide is a polypeptide of a parasite, wherein preferably said pathogen is selected from the group consisting of: (a) Toxoplasma spp.; (b) Plasmodium falciparum; (c) Plasmodium vivax; (d) Plasmodium ovale; (e) Plasmodium malariae; (f) Leishmania; (g) Schistosoma and (h) Nematodes. Preferably, said antigenic polypeptide is derived from Plasmodium falciparum or Plasmodium Vivax (SEQ ID NO: 88).

In a further preferred embodiment, said antigenic polypeptide is a polypeptide of a bacterium, wherein preferably said bacterium is selected from the group consisting of: (a) Chlamydia (b) Streptococccus; (c) Pneumococcus; (d) Staphylococcus; (e) Salmonella; (f) Mycobacteria; (g) Clostridia (h) Vibrio (i) Yersinia (k) Meningococcus (l) Borrelia.

Lyme disease is the most prevalent tick-born disease in Europe and North America, with about 400,000 registered cases annually. Disease may have different complications—pain in joints, neurological disorders, symptoms like multiple sclerosis and arthritis. Although the disease can be cured with antibiotics, symptoms may persist for years even after antibiotic treatment. Currently, no vaccine against Lyme disease is available in the market. In 1998, SmithKline Beecham Biologicals (now part of GlaxoSmithKline) developed LYMErix anti-Lyme vaccine, but it was removed from the market due to complaints about side-effects and multiple lawsuit cases. Therefore, at a global scale there is a need for a new, efficient and safe anti-Lyme vaccine. Borrelia genus bacteria, which cause Lyme disease, have many different proteins, located on their surface, creating an immune response against which may kill the pathogen. This approach was used in the Lymerix vaccine, which consisted of outer surface protein OspA. Since then, several other surface proteins of Borrelia burgdorferi have been tried as vaccine candidates, but none of them have reached the market so far. Borrelia species produce a number of surface proteins, which help to evade the destruction of bacteria by the complement system of the host. So-called CRASPs (complement regulator-acquiring proteins) are able bind complement regulator factor H (CFH) and CFH-like protein-1 (CFHL-1), which both inhibit complement activation and formation of membrane attack complex. CspZ is one of CRASPs, being able to bind both CFH and CFHL-1. Therefore, anti-CspZ antibodies would not only mark the surface of bacteria for attack of the immune system, but also reduce the ability of bacteria to avoid the complement.

Thus, in another preferred embodiment, said antigenic polypeptide is CspZ protein from Borrelia burgdorferi. In a very preferred embodiment, said antigenic polypeptide comprises, or preferably consists of SEQ ID NO:89 or an amino acid sequence having a sequence identity of at least 90%, preferably of at least 95%, with SEQ ID NO:89. Preferably, said antigenic polypeptide comprises, or preferably consists of SEQ ID NO:89. In another very preferred embodiment, said antigenic polypeptide consists of SEQ ID NO:89.

In a very preferred embodiment, said antigenic polypeptide comprises, or preferably consists of SEQ ID NO:90 or an amino acid sequence having a sequence identity of at least 90%, preferably of at least 95%, with SEQ ID NO:90. Preferably, said antigenic polypeptide comprises, or preferably consists of SEQ ID NO:90. In another very preferred embodiment, said antigenic polypeptide consists of SEQ ID NO:90.

As a consequence, the inventive modified virus-like particle of RNA bacteriophage AP205 comprising CspZ protein as the antigenic polypeptide can be useful as vaccine for protecting from Lyme borreliosis.

In a further preferred embodiment said antigenic polypeptide is a viral antigen, wherein preferably said viral antigen is a polypeptide derived from a virus selected from the group consisting of: (a) Retrovirus, preferably HIV; (b) Influenza virus, preferably influenza A M2 extracellular domain or HA or HA globular domain; (c) a polypeptide of Hepatitis B virus, preferably preS1; (d) Hepatitis C virus; (e) HPV, preferably HPV16E7; (f) RSV; (g) Coronavirus, preferably SARS-CoV-1, SARS-CoV-2, MERS, further preferably SARS-CoV-2; (h) Flavivirus, preferably Dengue virus, Zika Virus, West Nile Virus and Hand Foot and Mouth Disease Virus, and further preferably ectodomain III (ED3) from E protein of Dengue fever virus serotype 1; (i) Alphavirus, preferably Chikungunya; (k) Herpesvirus, preferably CMV; (1) Rotavirus. In a further preferred embodiment, said antigenic polypeptide is the derived from RSV.

In a further very preferred embodiment, said antigenic polypeptide is the derived from Dengue virus. Dengue fever is a vector-borne tropical disease, caused by Dengue fever virus. Each year about 390 million cases occur worldwide. Symptoms include fever, headache, vomiting, pain in joints and muscle and characteristic skin rash. In rare cases illness progresses to Dengue haemorrhagic fever, which is a life threatening condition, causing around 40,000 deaths worldwide annually. The first and only dengue vaccine that successfully completed clinical development has been withdrawn from the market in many countries due to safety concerns. Therefore, there still is a need for a safe dengue vaccine.

Envelope (E) protein is found on the surface of mature dengue virus particles and it is composed of three ectodomains EDI, EDII, EDIII (ED3) and a transmembrane region. It has been shown previously that ED3 alone results in production of high levels of EDIII-specific neutralizing antibodies. Therefore, ED3 could be used in fusion with the tandem dimers leading to the inventive modified VLPs as an efficient vaccine.

Thus, in another preferred embodiment, said antigenic polypeptide is derived, preferably is, from ectodomain III (ED3) from E protein of Dengue fever virus. In another preferred embodiment, said antigenic polypeptide is derived from ectodomain III (ED3) from E protein of Dengue fever virus serotype 1. Thus, in another preferred embodiment, said antigenic polypeptide is ectodomain III (ED3) from E protein of Dengue fever virus serotype 1. In a very preferred embodiment, said antigenic polypeptide comprises, or preferably consists of SEQ ID NO:91 or an amino acid sequence having a sequence identity of at least 90%, preferably of at least 95%, with SEQ ID NO:91. Preferably, said antigenic polypeptide comprises, or preferably consists of SEQ ID NO:91. In another very preferred embodiment, said antigenic polypeptide consists of SEQ ID NO:91.

In a very preferred embodiment, said antigenic polypeptide comprises, or preferably consists of SEQ ID NO:92 or an amino acid sequence having a sequence identity of at least 90%, preferably of at least 95%, with SEQ ID NO:92. Preferably, said antigenic polypeptide comprises, or preferably consists of SEQ ID NO:92. In another very preferred embodiment, said antigenic polypeptide consists of SEQ ID NO:92.

In a further preferred embodiment, said antigenic polypeptide comprises, or preferably consists of, position 9 to 99, position 9 to 109 or position 9 to 112 of SEQ ID NO:92 or an amino acid sequence having a sequence identity of at least 90%, preferably of at least 95%, with SEQ ID NO:92. Preferably, said antigenic polypeptide comprises, or preferably consists of, position 9 to 99, position 9 to 109 or position 9 to 112 of SEQ ID NO:92. In another very preferred embodiment, said antigenic polypeptide consists of position 9 to 99, position 9 to 109 or position 9 to 112 of SEQ ID NO:92.

In a preferred embodiment, said antigenic polypeptide is the extracellular domain of Influenza A virus M2 protein, or an antigenic fragment thereof. In a preferred embodiment said antigenic polypeptide comprises or preferably consists of the extracellular domain of the Influenza A virus M2 protein, wherein preferably said extracellular domain of the Influenza A virus M2 protein is SEQ ID NO:93. In another preferred embodiment, said antigenic polypeptide is the globular domain of Influenza virus. In another preferred embodiment, said antigenic polypeptide comprises the protease cleavage site of HA Influenza virus.

In a preferred embodiment, said antigenic polypeptide is a receptor binding domain (RBD) of a coronavirus (CoV), or a fragment thereof. In another preferred embodiment, said antigenic polypeptide is the receptor binding domain (RBD), preferably the receptor binding motif (RBM), of a spike (S) protein of a human coronavirus (HCoV), or a fragment thereof, wherein said HCoV is selected from SARS-CoV-2, SARS-CoV, MERS-CoV, HCoV-229E, HCoV-NL63, HCoV-OC43 and HCoV-HKU1, preferably from SARS-CoV-2, SARS-CoV and MERS-CoV, and again further preferably from SARS-CoV-2.

In a very preferred embodiment, said antigenic polypeptide comprises, or preferably consists of the amino acid sequence selected from SEQ ID NO:94, SEQ ID NO:95, SEQ ID NO:96, SEQ ID NO:97, and an amino acid sequence having a sequence identity of at least 80%, preferably of at least 90%, further preferably of at least 95% with any of SEQ ID NO:94, SEQ ID NO:95, SEQ ID NO:96 and SEQ ID NO:97.

In a very preferred embodiment, said antigenic polypeptide comprises, or preferably consists of SEQ ID NO:94 or an amino acid sequence having a sequence identity of at least 90%, preferably of at least 95%, with SEQ ID NO:94. Preferably, said antigenic polypeptide comprises, or preferably consists of SEQ ID NO:94. In another very preferred embodiment, said antigenic polypeptide consists of SEQ ID NO:94.

In a very preferred embodiment, said antigenic polypeptide comprises, or preferably consists of SEQ ID NO:95 or an amino acid sequence having a sequence identity of at least 90%, preferably of at least 95%, with SEQ ID NO:95. Preferably, said antigenic polypeptide comprises, or preferably consists of SEQ ID NO:95. In another very preferred embodiment, said antigenic polypeptide consists of SEQ ID NO:95.

In a further very preferred embodiment, said fusion protein is selected from the group consisting of SEQ ID NO:12, SEQ ID NO:16, SEQ ID NO:21, SEQ ID NO:24, SEQ ID NO:101, SEQ ID NO:106 and SEQ ID NO:110.

In a further aspect the invention provides the modified AP205 virus-like particle of the invention for use as a medicament.

In a further aspect the invention provides a vaccine comprising or alternatively consisting of the modified AP205 virus-like particle of the invention. Encompassed are vaccines wherein said modified AP205 VLPs comprise any one of the technical features disclosed herein, either alone or in any possible combination. In one embodiment the vaccine further comprises an adjuvant. In a further embodiment the vaccine is devoid of an adjuvant. In a preferred embodiment said vaccine comprises an effective amount of the composition of the invention.

In a further aspect, the invention relates to a pharmaceutical composition comprising: (a) a modified AP205 VLP of the invention or a vaccine of the invention; and (b) a pharmaceutically acceptable carrier, diluent and/or excipient. Said diluent includes sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Pharmaceutical compositions of the invention may be in a form which contain salts, buffers, adjuvants, or other substances which are desirable for improving the efficacy of the conjugate. Examples of materials suitable for use in preparation of pharmaceutical compositions are provided in numerous sources including Remington's Pharmaceutical Sciences (Osol, A, ed., Mack Publishing Co., (1990)). In one embodiment said pharmaceutical composition comprises an effective amount of the vaccine of the invention.

A further aspect of the invention is a method of immunization an animal or a human comprising administering a modified AP205 VLP of the invention, a vaccine of the invention, or a pharmaceutical composition of the invention to said animal or human. In a preferred embodiment said method comprises administering a modified AP205 VLP of the invention to said animal or human. A further aspect of the invention is a method of immunization an animal or a human comprising administering a modified AP205 VLP of the invention to said animal or human. A further aspect of the invention is a method of immunization an animal comprising administering a modified AP205 VLP of the invention to said animal. A further aspect of the invention is a method of immunization a human comprising administering a modified AP205 VLP of the invention to said human.

A further aspect of the invention is the use of the modified AP205 VLP of the invention, the vaccine of the invention, or the pharmaceutical composition of the invention in the manufacture of a medicament for the treatment or prevention of a disease or a disorder in an animal or in a human. A further aspect of the invention is an use of the modified AP205 VLP of the invention in the manufacture of a medicament for the treatment or prevention of a disease or a disorder in an animal. A further aspect of the invention is an use of the modified AP205 VLP of the invention in the manufacture of a medicament for the treatment or prevention of a disease or a disorder in a human.

A further aspect of the invention is a method of treating or preventing a disease or a disorder in an animal said method comprising administering a modified AP205 VLP of the invention, a vaccine of the invention, or a pharmaceutical composition of the invention to said animal, wherein preferably said animal can be a human. In a further preferred embodiment said modified AP205 VLP, said vaccine, or said pharmaceutical composition is administered to said animal subcutaneously, intravenously, intradermally, intranasally, orally, intranodal or transdermally.

A further aspect of the invention is a method of treating or preventing a disease or a disorder in an animal said method comprising administering a modified AP205 VLP of the invention to said animal, wherein preferably said animal can be a human. In a further preferred embodiment said modified AP205 VLP is administered to said animal subcutaneously, intravenously, intradermally, intranasally, orally, intranodal or transdermally. A further aspect of the invention is a method of treating or preventing a disease or a disorder in a human said method comprising administering a modified AP205 VLP of the invention to said human. In a further preferred embodiment said modified AP205 VLP is administered to said human subcutaneously, intravenously, intradermally, intranasally, orally, intranodal or transdermally.

In a further very preferred embodiment, said disease or disorder is selected from the group consisting of an allergy, a cancer, an autoimmune disease, an inflammatory disease, an infectious disease.

In a further very preferred embodiment, said disease or disorder is selected from the group consisting of RA, MS, Psoriasis, asthma, Crohns, Colitis, COPD, diabetes, neurodermatitis (allergic dermatitis), Alzheimer's disease, Parkinson's disease, influenza A virus infection, Dengue virus infection, corona virus infection, preferably SARS-CoV2 infection, African Swine Fever Virus infection, Lyme disease preferably Lyme borreliosis, malaria, RSV infection.

In a further very preferred embodiment, said disease or disorder is an inflammatory disease. In a further very preferred embodiment, said disease or disorder is an inflammatory disease selected from RA, MS, Psoriasis, asthma, Crohns, Colitis, COPD, diabetes, neurodermatitis (allergic dermatitis).

In a further very preferred embodiment, said disease or disorder is an infectious disease. In a further very preferred embodiment, said disease or disorder is an infectious disease selected from influenza A virus infection, Dengue virus infection, African Swine Fever Virus infection, SARS-CoV2 infection, malaria, RSV infection. In a further very preferred embodiment, said disease or disorder is a Dengue virus infection, a corona virus infection, preferably a SARS-CoV2 infection, Lyme disease, preferably Lyme borreliosis and atopic dermatitis preferably canine atopic dermatitis.

### EXAMPLES

### Example 1

### Cloning, Expression and Purification of Recombinant AP205 Tandem Dimer VLPs with CspZ in N- or in C-Terminal Ends

### Cloning of the AP205 Tandem Dimer

AP205 tandem dimer was cloned in pET-Duet1 vector (Novagen) in two steps. In the first step PCR fragment containing AP205 gene with Nco I and BamH I restriction sites for cloning in pET-Duet1 was generated with upstream primer: APncof 5′-tacaccatggcaaataagccaatg-3′ (SEQ ID NO:1) and downstream primer: APbamr 5′-tacattaggatccagcagtagtatcagacgata-3′ (SEQ ID NO:2) and template plasmid, containing AP205 coat protein gene sequence (NCBI Reference Sequence: NC_002700.2; SEQ ID NO:3).The PCR product was digested with NcoI and BamHI and cloned in the same restriction sites into pET-Duet1. In the second step PCR fragment containing AP205 gene with BamH I and Pst I restriction sites was generated with upstream primer: APbamf 5′-tacaggatccgcaaataagccaatgcaacc-3′ (SEQ ID NO:4) and downstream primer APnher 5′-tacactgcagttagctagcagtagtatcagacgatac-3′ (SEQ ID NO:5) and template plasmid, containing AP205 coat protein gene sequence (NCBI Reference Sequence: NC_002700.2; SEQ ID NO:3). The PCR product was digested with BamHI and Pst I and cloned in the same restriction sites into the plasmid, obtained in the first step. As a result, expression plasmid pET-Duet1-AP205TD (SEQ ID NO:6) was obtained, encoding AP205 coat protein tandem dimer AP205TD (SEQ ID NO:7). The AP205TD sequence contains two AP205 coat protein genes, separated by a two amino acid glycine-serine (GS) linker and possessing an extra serine residue at the C-terminus. GS linker was added to allow some flexibility between both coat protein halves and C-terminal serine is a consequence of engineered NheI restriction site to allow insertions of foreign sequences.

### Cloning of the CspZ Gene in the N-Terminus of AP205 Tandem Dimer

PCR fragment, containing CspZ gene sequence of Borrelia burgdorferi B31 strain (NCBI Reference Sequence: NC_001853.1; SEQ ID NO:8) and NcoI restriction sites in both ends was generated with upstream primer CspZf 5′-tacaccatggcaagaaatattaatgagcttaaaatt-3′ (SEQ ID NO:9), downstream primer CspZr 5′-cataccatggctaataaagtttgcttaatagctttat-3′ (SEQ ID NO:10) and template plasmid, containing CspZ gene of Borrelia burgdorferi B31 strain (NCBI Reference Sequence: NC_001853.1 SEQ ID NO:8). The PCR product was cleaved with NcoI and cloned in the same restriction site of plasmid pET-Duet1-AP205TD (SEQ ID NO:6). In this way we obtained the expression plasmid pET-Duet1-CspZ-AP205TD (SEQ ID NO:11), encoding fusion protein CspZ-AP205TD (SEQ ID NO:12).

### Cloning of the CspZ Gene in the C-Terminus of AP205 Tandem Dimer

PCR fragment, containing CspZ gene sequence and NheI restriction sites in both ends was generated with upstream primer: CspZf2 5′-tacagctagcagaaatattaatgagcttaaatt-3′(SEQ ID NO:13) and downstream primer CspZr2 5′-catagctagctaataaagtttgcttaatagctttat-3′ (SEQ ID NO:14) and template plasmid, containing CspZ gene of Borrelia burgdorferi B31 strain (NCBI Reference Sequence: NC_001853.1 SEQ ID NO:8). The PCR product was cleaved with NheI and cloned in the same restriction site of plasmid pET-Duet1-AP205TD (SEQ ID NO:6). In this way we obtained the expression plasmid pET-Duet1-AP205TD-CspZ (SEQ ID NO:15), encoding fusion protein AP205TD-CspZ (SEQ ID NO:16).

### Production of Recombinant CspZ-AP205TD and AP205TD-CspZ Fusion Proteins

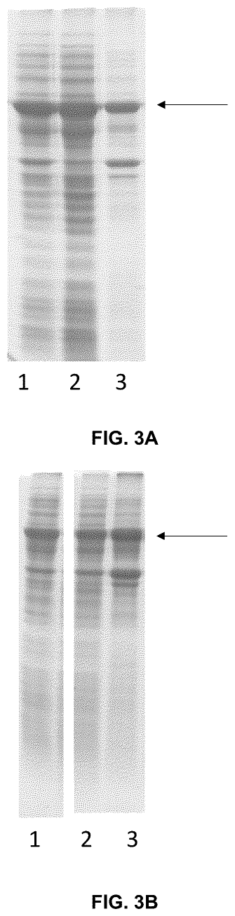

E. coli cells BL21(DE3) were transformed with plasmid pET-Duet1-AP205TD-CspZ or pET-Duet1-CspZ-AP205TD. 5 ml of LB liquid medium with 20 μg/ml ampicillin were inoculated with a single colony and incubated at 37° C. for 16-24 h without shaking. The prepared inoculum was diluted 1:100 in 100-300 ml of LB medium, containing 20 μg/ml ampicillin and incubated at 37° C. overnight without shaking. The resulting second inoculum was diluted 1:50 in 2×TY medium and incubated with shaking at 37° C. to an OD 600 of 0.8-1.0. Then the expression was induced with 0.2 mM (final concentration) IPTG. Incubation was continued on the rotary shaker at 20° C. for 18-20 h. Cells were harvested by centrifugation and frozen at −20° C. The presence of produced recombinant proteins was verified by SDS-PAGE analysis (FIG. 3A and FIG. 3B).

### Purification of Recombinant CspZ-AP205TD Protein VLPs

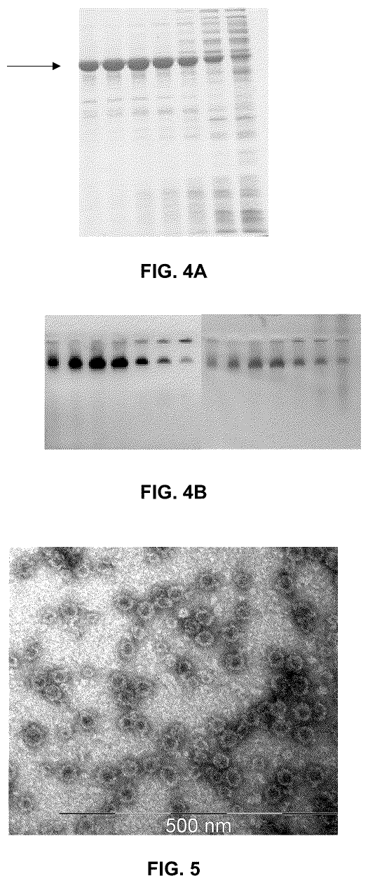

Frozen cells were thawed and resuspended in a lysis buffer (4 ml of buffer per 1 g wet cells). The mixture was sonicated at 24 kHz and 4° C. for 10 min, with on/off intervals of 0.5 sec. After sonification, urea was added to a final concentration of 1M and lysate incubated on ice for 30 min. The lysate was then centrifuged for 30 minutes at 10000 g. The pooled supernatant was loaded on a Sepharose 4FF column and proteins were eluted with PBS (Akta Prime Plus, GE Healthcare). The fractions were analyzed by 15% PAGE-SDS and native agarose gel (1% agarose in TAE buffer, stained with 0.05% ethidium bromide). Fractions with visible VLP bands in agarose gel were pooled and concentrated with 15 ml 100 kDa cutoff Amicon concentration filters (Millipore). Concentrated VLPs were loaded on Superose6 gel filtration column and eluted with PBS (Akta Prime Plus, GE Healthcare). The fractions were analyzed by 15% PAGE-SDS and native agarose gel and VLP-containing fractions, containing purified CspZ-AP205TD protein VLPs pooled. See FIG. 4a and FIG. 4B. For electron microscopy, the protein samples were adsorbed on carbon-Formvar-coated copper grids and negatively stained with 1% uranyl acetate aqueous solution. The grids were examined with a JEM-1230 electron microscope (JEOL Ltd., Tokyo, Japan) at 100 kV. The presence of VLPs was confirmed by electron microscopy (FIG. 5). The outcome was about 4.5-5.0 mg of purified protein per gram of wet cells.

### Purification of Recombinant AP205TD-CspZ Protein VLPs

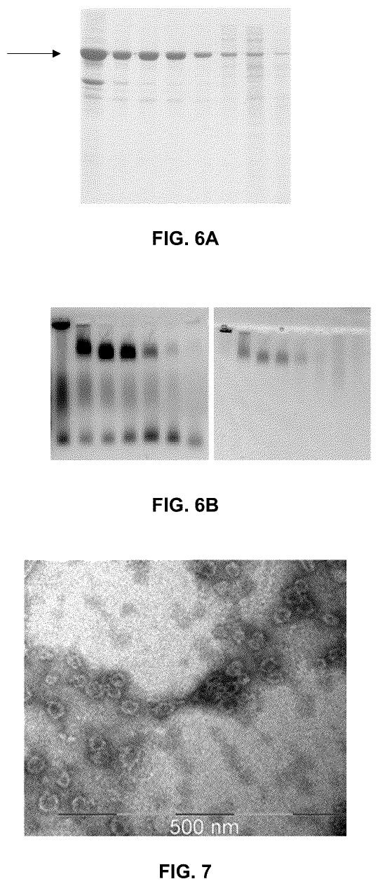

Frozen cells were thawed and resuspended in a lysis buffer (4 ml of buffer per 1 g wet cells). The mixture was sonicated at 24 kHz and 4° C. for 10 min, with on/off intervals of 0.5 sec. After sonication, urea was added to a final concentration of 1M and lysate incubated on ice for 30 min. The lysate was then centrifuged for 30 minutes at 10000 g. Clarified cell lysate was precipitated for one hour with 20% saturated ammonium sulfate. The precipitate was collected by centrifugation and dissolved in a lysis buffer. Clarified supernatant was loaded on a Sepharose 4FF column and proteins were eluted with PBS (Akta Prime Plus, GE Healthcare). The fractions were analyzed by 15% PAGE-SDS and native agarose gel (see FIG. 6A and FIG. 6B). Fractions with visible VLP bands in agarose gel were pooled and concentrated with 15 ml 100 kDa cutoff Amicon concentration filters (Millipore). The presence of VLPs was confirmed by electron microscopy (FIG. 7). The outcome was about 0.6 mg of purified protein per gram of wet cells.

### Solutions and Buffers for Example 1

Lysis buffer: 50 mM Tris-HCl pH 8.0, 150 mM NaCl, 0.1% tritonX100, 1 mM PMSF.
PBS: 137 mM NaCl, 2.7 mM KCl, 10 mM Na₂HPO₄, 1.8 mM KH₂PO4.

Thus, CspZ antigen was inserted both in C- and N-terminal parts of AP205TD and in both cases soluble VLPs were formed. We also attempted to insert CspZ antigen in N- and C-terminal parts of single (non-tandem) coat protein of AP205 but failed to obtain VLPs in both cases (FIG. 3).

### Example 2

### Cloning, Expression and Purification of Recombinant AP205 Tandem Dimer VLPs with Dengue Virus Serotype 1 E Protein Ectodomain EDIII in N-Terminal End

### Cloning of the ED3 in N-Terminal End of AP205 Tandem Dimer.

PCR fragment, containing ED3 gene sequence and Nco I restriction sites in both ends was generated with upstream primer: ED3f 5′-gatataccatggataaactgaccctgaaag-3′(SEQ ID NO:17) and downstream primer ED3r 5′-atttgccatggcaccgctgcccattttgccaat-3′ (SEQ ID NO:18) and template plasmid, containing ED3 gene of Dengue serotype 1 (SEQ ID NO:19). The PCR product was cleaved with NcoI and cloned in the same restriction site of plasmid pET-Duet1-AP205TD (SEQ ID NO:6). In this way we obtained the expression plasmid pET-Duet1-ED3-AP205TD (SEQ ID NO:20), encoding fusion protein ED3-AP205TD (SEQ ID NO:21).

### Production of Recombinant ED3-AP205TD VLPs

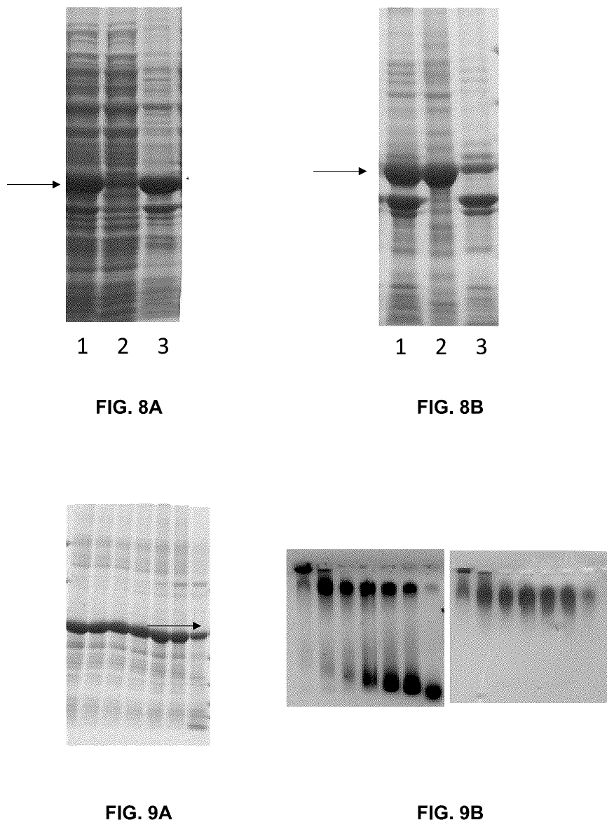

E. coli cells BL21(DE3) were transformed with plasmid pET-Duet1-ED3-AP205TD. 5 ml of LB liquid medium with 20 g/ml ampicillin were inoculated with a single colony and incubated at 37° C. for 16-24 h without shaking. The prepared inoculum was diluted 1:100 in 100-300 ml of LB medium, containing 20 g/ml of ampicillin and incubated at 37° C. overnight without shaking. The resulting second inoculum was diluted 1:50 in 2×TY medium and incubated with shaking at 37° C. to an OD 600 of 0.8-1.0. Then the expression was induced with 0.2 mM IPTG (final concentration). Incubation was continued on the rotary shaker at 20° C. for 18-20 h. Cells were harvested by centrifugation and frozen at −20° C. The presence of produced recombinant proteins was verified by SDS-PAGE analysis (FIG. 8A).

### Purification of Recombinant ED3-AP205TD VLPs

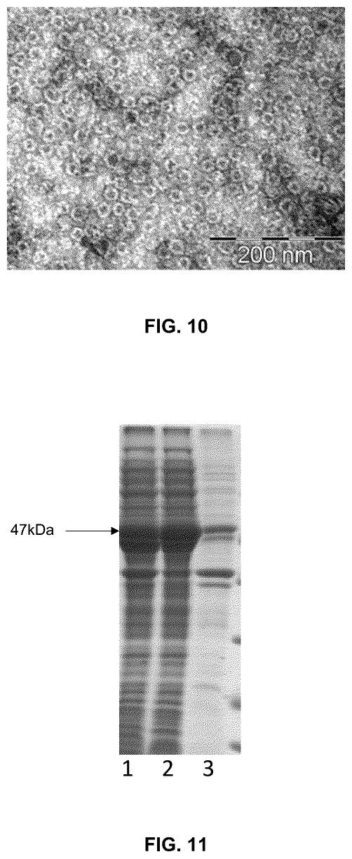

Frozen cells were thawed and resuspended in a lysis buffer (10 ml buffer per 1 g wet cells). The mixture was sonicated at 24 kHz for 10 min at +4° C., with on/off intervals of 0.5 sec. Cell lysate was centrifuged for 30 min at 10000 g at 4° C. and supernatant discarded. The pellet was washed 3 times in a lysis buffer (same volume as taken for cell lysis). A single step of washing was performed in the same way as cell lysis—pellet was resuspended in the lysis buffer, sonicated, centrifuged and supernatant discarded. The pellet was further resuspended in IB solubilization buffer and incubated for 16 h at 4° C. on end-over-end rotator (30 rpm). Next, suspension was centrifuged for 30 min at 10000 g at 4° C. The obtained supernatant was further dialyzed against 100 volumes of RB I buffer for 24 h at 4° C., subsequently—100 volumes of RB II buffer for 24-36 h at 4° C., and finally against 100 volumes of PBS at 4° C. The dialyzed supernatant was further centrifuged for 30 min, at 10000 g at 4° C. and pellet discarded. The refolding efficiency was assessed by SDS-PAGE electrophoresis (FIG. 8B). Supernatant was loaded on a Sepharose 4FF column and proteins were eluted with PBS (Akta Prime Plus, GE Healthcare). The fractions were analyzed by 15% PAGE-SDS (FIG. 9A) and native agarose gel (1% agarose in TAE buffer, stained with 0.05% ethidium bromide) (FIG. 9B). Fractions with visible VLP bands in agarose gel were pooled and concentrated with 15 ml 100 kDa cutoff Amicon concentration filters (Millipore). The presence of VLPs was confirmed by electron microscopy (FIG. 10). The outcome was about 2 mg of purified protein per gram of wet cells.

### Solutions and Buffers for Example 2

Lysis buffer: PBS with 0.1% Triton X100
IB solubilization buffer: 8 M urea, 50 mM Tris-HCl pH 8.0, 150 mM NaCl
RB I buffer: 2 M urea, 100 mM PB pH 8.0, 0.5 M arginine, 5 mM reduced glutathione, 0.5 mM oxidized glutathione
RB II buffer: 100 mM PB pH 8.0, 0.5 M arginine, 5 mM glutathione reduced, 0.5 mM glutathione oxidized.

Thus, the generation of fusion proteins with ED3 was successfully achieved regardless of ED3 placement at the N- or C-terminus of AP205TD (only N-terminal fusion described in detail). The expression yielded first insoluble products, which formed soluble modified AP205 VLPs after re-folding.

### Example 3

### Cloning, Expression and Purification of Recombinant AP205 Tandem Dimer VLPs with Canine IL-33 in C-Terminal End and Immunization of Mice to Generate Binding and Neutralizing Antibodies

Cloning of the cIL33 in C-Terminal End of AP205 Tandem Dimer.

cIL33 codon-optimized gene (SEQ ID NO:22) was purchased from BioCat (General Biosystems, Inc) in the form of a plasmid. The IL33 was cut from the plasmid with restriction endonucleases Nhe I and Pst I and the resulting DNA fragment, containing the cIL33 gene sequence cloned into the same restriction sites of plasmid pET-Duet1-AP205TD (SEQ ID NO:6). In this way we obtained the expression plasmid pET-Duet1-AP205TD-cIL33 (SEQ ID NO:23), encoding fusion protein AP205TD-cIL33 (SEQ ID NO:24).

### Production of Recombinant AP205TD-cIL33 VLPs

E. coli cells BL21(DE3) were transformed with plasmid pET-Duet1-AP205TD-cIL33. 5 ml of LB liquid medium with 20 g/ml ampicillin were inoculated with a single colony and incubated at 37° C. for 16-24 h without shaking. The prepared inoculum was diluted 1:100 in 100-300 ml of LB medium, containing 20 g/ml of ampicillin and incubated at 37° C. overnight without shaking. The resulting second inoculum was diluted 1:50 in 2×TY medium and incubated with shaking at 37° C. to an OD 600 of 0.8-1.0. Then the expression was induced with 0.2 mM IPTG (final concentration). Incubation was continued on a rotary shaker at 20° C. for 18-20 h. Cells were harvested by centrifugation and frozen at −20° C. The presence of induced recombinant protein of ˜47 kDa was verified by SDS-PAGE analysis (FIG. 11).

### Purification of Recombinant AP205TD-cIL33 VLPs

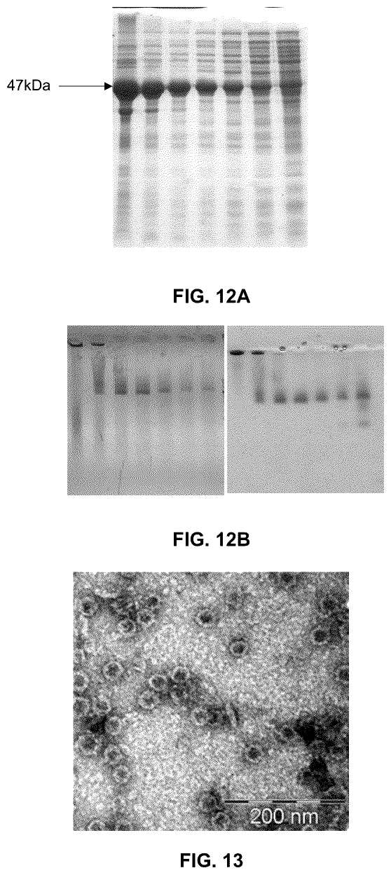

Frozen cells were thawed and resuspended in a 50 mM Tris-HCl pH 8.0, 150 mM NaCl, 0.1% tritonX100, 1 mM PMSF (4 ml of buffer per 1 g wet cells). The mixture was sonicated at 24 kHz and 4° C. for 10 min, with on/off intervals of 0.5 sec. After sonification the lysate was centrifuged for 30 minutes at 10000 g and the pellet discarded. Ammonium sulphate was added to supernatant to 40% saturation, and the solution centrifuged for 30 minutes at 10000 g and the supernatant discarded. The pellet was dissolved in PBS and loaded onto a Sepharose 4FF column. Proteins were eluted with PBS (Akta Prime Plus, GE Healthcare) and fractions analyzed by 15% PAGE-SDS and native agarose gel (1% agarose in TAE buffer, stained with 0.05% ethidium bromide) (FIG. 12). Fractions with visible VLP bands in agarose gel were pooled and concentrated using a 100 kDa cutoff Amicon concentration filters (Millipore). The presence of VLPs was confirmed by electron microscopy (FIG. 13). The yield was 4.5-5.0 mg of purified protein per gram of wet cells.

Thus, fusion of cIL33 to C-terminus of AP205TD was demonstrated to result in soluble 47 kDa fusion proteins (comprising AP205TD and cIL-33) which assembled into integral 25-30 nm modified AP205 VLPs in accordance with the present invention and composed of about 90×tandem dimers of AP205 each displaying 1×cIL33.

### Immunization of Mice to Generate Binding and Neutralizing Antibodies

Groups of five female Balb/c mice were injected via the intra-peritoneal route with 150 ul of AP205TD-cIL33 (30 ug/dose) VLPs formulated with 15 ug Quil-A® adjuvant (Brenntag Biosector) in phosphate buffered saline or with phosphate buffered saline alone. After 14 days, the mice were injected again with the same formulations. Mice were bled on days 0 (pre-immune), day 14, 28, 42, 56, and 98. Sera were analyzed for IL-33-specific binding IgG antibodies by ELISA and for neutralizing antibodies using a bioassay.

For the ELISA, NUNC plates were coated with canine IL-33 (recombinantly produced in E. coli) in PBS with a concentration of 1 μg/ml overnight at 4° C. The plates were blocked with Superblock (Invitrogen). A serial dilution of the sera was performed in order to calculate OD50. Values. OD50 describes the reciprocal of the dilution, which reaches half of the maximal OD value. Antibodies of the subtype gamma (IgG) specific for cIL-33 were detected with an anti-mouse IgG antibody directly labeled to horseradish dish peroxidase (HRPO) purchased from Jackson. The conversion of o-phenylenediamine dihydrochloride (OPD) by the HRPO was measured as color reaction at 450 nm, which was stopped by adding 5% sulfuric acid (H2SO4) after 7 minutes incubation.

For the neutralization assay, HEK-Blue IL-33 cells from InvivoGen (hkb-hil33) were used. In this assay IL33 signaling leads to the activation of NF-kB and Aβ-1 pathways, which result in the production of a secreted alkaline phosphatase reporter which can be measured in the cell supernatant. The cell culture and the neutralization setup were performed according to the manufacturer's instructions with the exceptions that on day one of the assay the volume per well was 100 ul and on day two, 40 ul of HEK-Blue IL-33 cell supernatant was added to 160 ul QUANTI-Blue solution per well. Serial diluted mice sera were incubated in the presence of 5 ng/mL canine IL-33 (Sino Biological 700005-DNAE) before addition to HEK-Blue IL-33 cells.

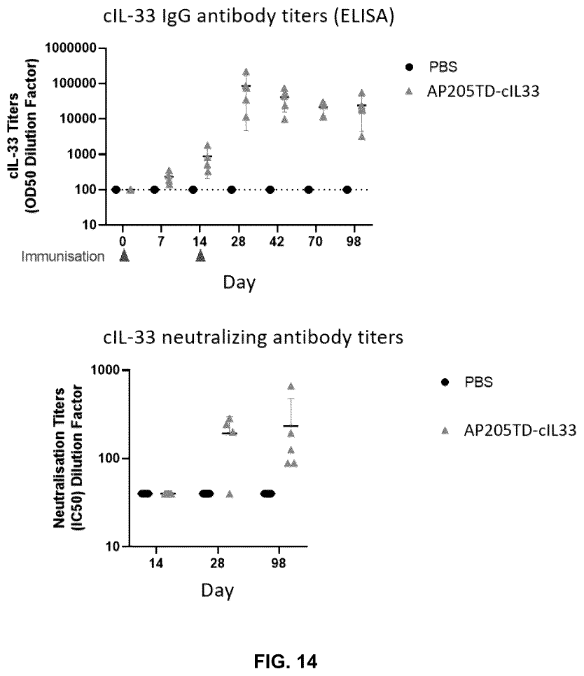

For mice receiving the AP205TD-cIL33 vaccine, canine IL-33 specific IgG antibodies were detected in sera collected on day 14; after a single immunization (FIG. 14, upper panel). Titers were substantially boosted by a second immunization administered on day 14. Peak titers were measured in day 28 sera and declined slowly over the course of the 98-day experiment. Neutralizing antibodies were detected in day 28 sera of 4 of 5 mice and in day 98 sera in 5/5 mice (FIG. 14, lower panel). The group receiving PBS had no detectable canine IL-33 binding or neutralizing antibodies.

Thus, the data show the AP205TD-cIL33 VLP vaccine was capable of inducing antibodies that were able to both bind and neutralize canine IL-33.

### Example 4

### Cloning, Expression and Purification of Recombinant AP205 Tandem Dimer VLPs with the RBM Domain of SARS-CoV-2 in C-Terminal End and Immunization of Mice to Generate Binding and Neutralizing Antibodies

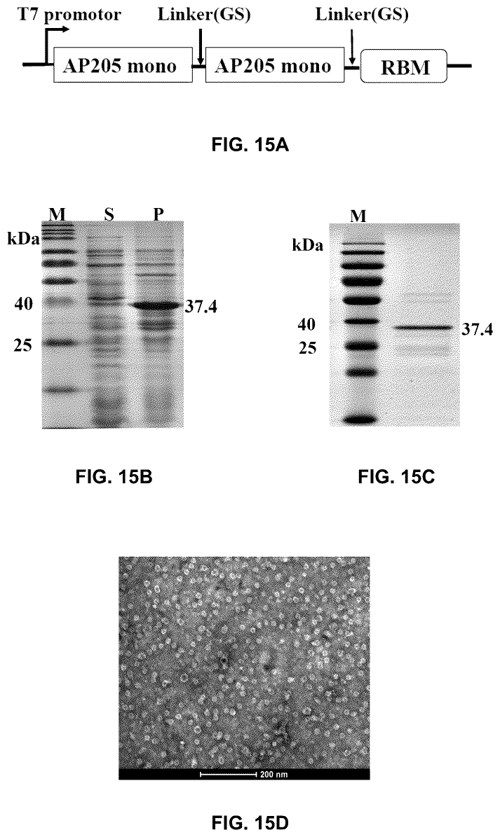

The AP205 coat protein dimer of the present invention can be utilized efficiently for generating a modified AP205 VLP in accordance with the present invention, and thus for a fusion vaccine against SARS-CoV-2. The exemplified prepared fusion protein described in this example is abbreviated for the sake of ease and named AP205-RBM (FIG. 15A).

### Cloning of the RBM Domain of SARS-CoV-2 at the C-Terminal End of AP205 Tandem Dimer

DNA encoding the Receptor Binding Motif (RBM) corresponding to residues 437-508 of SARS-CoV-2 Spike protein (GenBank accession number QIA98606.1, SEQ ID NO:95) was amplified by PCR with Q5@ High-Fidelity Master Mix (New England Biolabs, Ipswich, USA) using a codon optimized pUCIDT-SARS-CoV-2-RBD plasmid (SEQ ID NO:98) as template and fused at the C-terminus of AP205TD between the Bmt I and Hind III sites in the pETDuet-1-AP205 dimer plasmid (SEQ ID NO:6) with a C-terminal 6×His-tag. For PCR amplification the following primers were used: F: 5′-tctgatactactgctagcggatccaacagcaacaacc-3′ (SEQ ID NO:99) and R: 5′-attatgcggccgcaagctttagtgatggtgatggtgatgactagtatacggctgatag-3′ (SEQ ID NO:100). The corresponding PCR fragment was analyzed in 1.2% agarose gel and purified with GeneJet Gel Extraction kit (Thermio Scientific, USA). The PCR product and plasmid pETDuet-1-AP205 were digested with enzymes Bmt I and Hind III (Thermo Fischer Scientific, Waltham, Massachusetts) and ligated, resulting in plasmid pETDuet-1-AP205-RBM (SEQ ID NO:101). E. coli XL1-Blue host cells were used for cloning and plasmid amplification. After sequencing, plasmid were transformed into T7 Express Competent E. coli C2566 (High Efficiency) (New England Biolabs, Ipswich, USA).

### Expression of AP205-RBM

E. coli C2566 were grown in LB medium containing Ampicillin (100 μg/ml) on a rotary shaker (200 rpm) at 37° C. to an OD600 of 0.4-0.8. Following addition of 0.1 mM Isopropyl-β-D-thiogalactopyranoside the expression phase was performed at 16° C. for 16 h. The biomass was collected by low-speed centrifugation and frozen at −70° C. After thawing on ice, cells were suspended in 20 mM Tris-HCl pH 8.0 100 mM NaCl, 2 mM EDTA, 1 mM PMSF, 5% glycerol, and 0. 1% Triton X-100 and disrupted by ultrasonication. Insoluble and soluble proteins were separated by centrifugation. SDS-PAGE using a 12% gel showed the 37.4 kDa construct was expressed in an insoluble form (FIG. 15B).

### Protein Refolding and Purification

Cell pellets were, the pellets were resuspended in lysis buffer (above) by sonication and centrifuged for 20 min, 10,000 g at 4° C. The process was repeated for 4×. The pellet containing inclusion bodies was solubilized in 8 M urea, 20 mM Tris-HCl and 100 mM NaCl for 16 h at 4° C. on a rotating wheel. Following centrifugation for 20 min at 10,000 g, the supernatant was collected and sequentially dialyzed against 4M urea, 20 mM Tris-HCl, 0.5 M Arginine, 5 mM reduced Glutathione and 0.5 mM L-oxidized Glutathione for 24 h at 4° C., then 2M urea, 20 mM Tris-HCl, 0.5 M Arginine, 5 mM reduced Glutathione and 0.5 mM L-oxidized Glutathione) for 24 h at 4° C. and finally 20 mM Tris-HCl, 0.5 M Arginine, 5 mM reduced Glutathione and 0.5 mM L-oxidized Glutathione for 36 h at 4° C. After centrifugation for 20 min at 10,000 g at 4° C., the soluble refolded fusion protein which reassembles into VLPs was purified by HisTrap™ (GE Healthcare, Germany) and analyzed on 12% SDS-PAGE and electron microscopy (FIG. 15C and FIG. 15D respectively). For EM 2 μl of purified AP205-RBM protein (1 mg/ml) suspension for negative staining was adsorbed on glow discharged and carbon coated copper grids (Plano, Wetzlar; Germany) for 1 min at RT. After washing 3× with pure water, grids were stained with 2% uranyl acetate solution (Electron Microscopy Science, Hatfield, USA) for 30 seconds. Excess fluid was removed and samples examined at 80 kV with a transmission electron microscope (Tecnai Spirit, FEI, Hillsboro, USA) equipped with a digital camera (Veleta, Olympus, Munster, Germany)

In summary, expression of pETDuet-1-AP205-RBM resulted in large amounts of insoluble aggregates of AP205-RBM which could be easily denatured and refolded into soluble 37.4 kDa fusion proteins in accordance with the present invention which assembled into integral 25-30 nm modified AP205 VLPs composed of typically and preferably 90×tandem dimers of AP205 each displaying 1×RBM domain. (FIGS. 15B and 15C).

### Immune Response to AP205-RBM VLP Vaccine.

Vaccination regimen. Wild type Balb/c female mice were vaccinated subcutaneously (s.c.) with 100 μg AP205-RBM VLPs or AP205 VLPs in 100p PBS on day 0 and 28 and sera collected on days 0, 14, 21, 35 and 49. Sera from immunized mice were used to measure IgG antibodies capable of binding RBD and Spike proteins of SARS-CoV-2 (ELISA) and neutralizing antibodies (Pseudotype virus neutralization assay).

Enzyme-linked immunosorbent assay (ELISA). ELISA plates were coated overnight with 0.1 μg/ml and 1.0 μg/ml of S protein RBD or full spike protein (Sinobiological, Beijing, China) respectively. Plates were washed with PBS-0.01% Tween and blocked using 100 μl PBS-Casein 0.15% for 2 h in RT. For titration purposes, sera, initially diluted 1/20 then serially diluted ⅓, were added (100 ul per well) to the wells. Plates were incubated for 1 h at RT. After washing with PBS-0.01% Tween, goat anti-mouse IgG conjugated to Horseradish Peroxidase (HRP) (Jackson ImmunoResearch, West Grove, Pennsylvania) was added 1/2000 and incubated for 1 h at RT. Plates were developed and OD 450 reading was performed.

Neutralization assay. Vesicular stomatitis pseudotyped virus production has been described elsewhere (Whitt M. A., 2010, J. Virol Meth, 169 365-374) and used after modification to incorporate SARS-CoV-2_Spike and the TCID₅₀ was tested on HEK293T/17 cells transiently expressing ACE2 and transmembrane protease serine subtype 2 (TMPRSS2).

Neutralization assays were undertaken using 100×TCID₅₀ per well of 96-well plate. The virus was incubated for 1 hour at 37° C. (5% CO₂) along with the heat-inactivated serum, which was diluted over a range of 1:20-1:500. After which, 2×10⁴ HEK293T/17 cells transiently expressing ACE2 and TMPRSS2 were added to each well and the plate left to incubate (37° C., 5% CO₂) for a further 48 hours. The media was then discarded and the level of reporter gene activity assessed using a 50:50 mix of non-supplemented media: BrightGlo and a read in a GloMax Discover (Promega).

CPE-based assay: the capacity of the induced antibodies in neutralizing wild-type SARS-CoV-2 (SARS-CoV-2/ABS/NL20) was also performed. Serum samples were heat-inactivated for 30 min at 56° C. Two-fold serial dilutions were prepared starting at 1:20 up to 1:160. 100 TCID50 of the virus was added to each well and incubated for 37° C. for 1 h. The mixture has been added on a monolayer of Vero cells and incubated again for 37° C. for 4 days. Four days later the cells were inspected for cytopathic effect (CPE). The titer was expressed as the highest dilution that fully inhibits formation of CPE. Data were analyzed and presented as mean±SEM using GraphPad PRISM 8. P-values **P<0.01; *P<0.05.

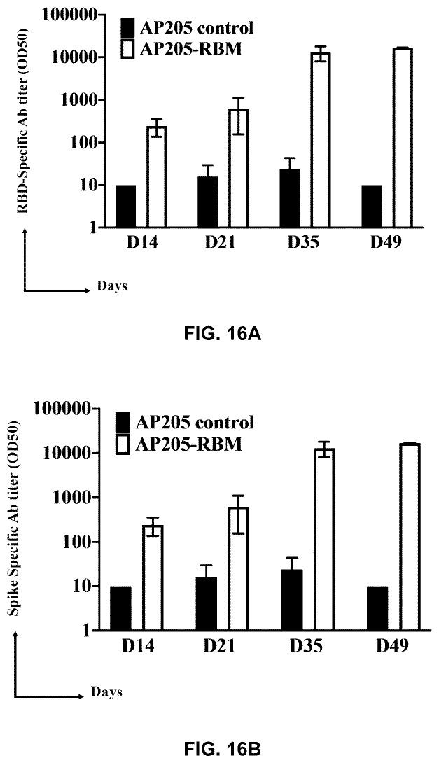

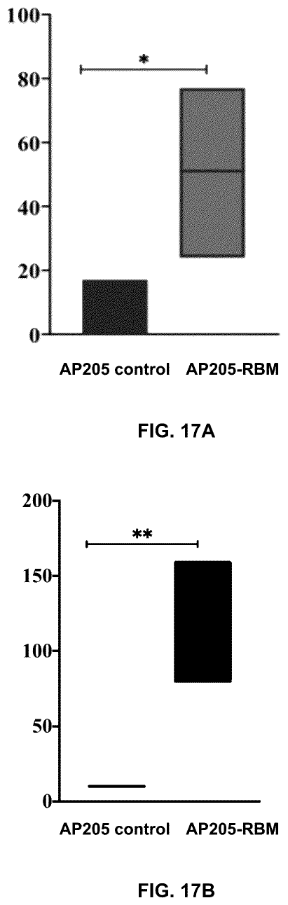

Results of the assessment of the immune response to AP205-RBM VLP. Immunization of naïve mice with AP205-RBM VLP vaccine resulted in an increase in RBD-specific (FIG. 16A) and Spike protein (FIG. 16B) specific IgG antibody titers 14 days after priming which were further increased following injection on day 28. No RBD-specific antibodies were detected in the mice vaccinated with AP205 as a control. These data show the IgG antibodies induced by the AP205-RBM VLPs in accordance with the present invention are capable of recognizing the larger eukaryotically expressed RBD domain from which the RBM was derived and the still larger spike protein which contains the RBD within a trimeric native structure. Moreover, the induced antibodies were able to neutralize a pseudotype vesicular stomatitis virus (17A) as well as SARS-CoV-2/ABS/NL20 (FIG. 17A and FIG. 17B) demonstrating that the AP205-RBM VLP is an effective vaccine candidate.

### Example 5

### Cloning, Expression and Purification of Recombinant AP205 Tandem Dimer VLPs with African Swine Fever Virus (ASFV) p12 Protein Surface Exposed Peptide in Both N- and C-Terminal End

### Cloning of the ASFV p12 in N- and C-Terminal Ends of AP205 Tandem Dimer.

In the first step, ASFV p12 protein surface exposed peptide (further—p12) gene was cloned into the N-terminal end of AP205 tandem dimer. Since the inserted peptide was relatively short, only 12 residues (SEQ ID NO:102), p12 gene was purchased from Metabion International AG (Germany) as two single stranded oligonucleotides. One oligonucleotide (SEQ ID NO:103) encoded p12 gene in forward orientation and the other one (SEQ ID NO: 104) in reverse orientation. The oligonucleotides were complementary to each other and had NcoI recognition sites at both ends. Both oligonucleotides were annealed into a single dsDNA fragment by mixing them together in equimolar amounts (30 mM each) and incubating at their melting temperature (88° C.) for 1 min. The obtained dsDNA fragment containing p12 gene sequence was cleaved with NeoI and cloned in the same restriction site of plasmid pET-Duet1-AP205TD. As a result, expression plasmid pET-Duet1-np12-AP205TD (SEQ ID NO: 105) encoding fusion protein np12-AP205TD (SEQ ID NO:106) was prepared. The np12-AP205TD sequence contains the AP205TD sequence (SEQ ID NO:7) and possessing an extra alanine residue at the N-terminus of the AP205TD sequence as a consequence of the cloning strategy.

Further, we used the obtained pET-Duet1-np12-AP205TD (SEQ ID NO:105) to insert p12 sequence into the C-terminal end of AP205 tandem dimer. For this, another two single stranded oligonucleotides encoding p12 were ordered from Metabion International AG (Germany). This time forward (SEQ ID NO:107) and reverse (SEQ ID NO:108) oligonucleotides contained NheI and PstI recognition sites. Double stranded DNA fragment encoding p12 protein was obtained as described above, except that the melting temperature was adjusted to 90° C. Then DNA fragment was cleaved with NheI and PstI and cloned into the same restriction sites of plasmid pET-Duet1-np12-AP205TD (SEQ ID NO:105). This way we obtained a new expression plasmid pET-Duet1-np12-AP205TD-cp12 (SEQ ID NO:109), encoding fusion protein np12-AP205TD-cp12 (SEQ ID NO:110). The presence of p12 sequence at both AP205 tandem dimer terminal ends was verified by Sanger sequencing.

Production of Recombinant np12-AP205TD-cp12 VLPs

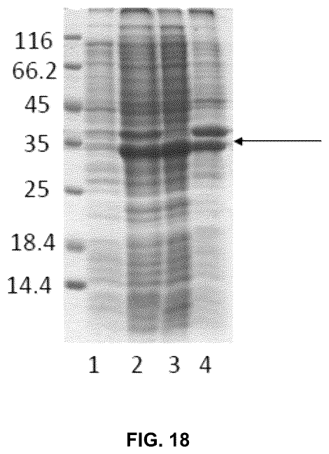

E. coli cells BL21(DE3) were transformed with plasmid pET-Duet1-np12-AP205TD-cp12. A single colony was put into 30 ml of LB liquid medium with 50 μg/ml of ampicillin and incubated at 37° C. for 16-24 h without shaking. The prepared inoculum was diluted 1:10 in 2×TY medium and incubated with shaking at 37° C. to an OD 600 of 0.8-1.0. Then the expression of the fusion protein was induced with 0.5 mM IPTG (final concentration). Incubation was continued on the rotary shaker at 20° C. for 18-20 h. Cells were harvested by centrifugation and frozen at −20° C. The presence of produced recombinant proteins was verified by SDS-PAGE analysis (FIG. 18).

### Purification of Recombinant Np12-AP205TD-Cp12 VLPs

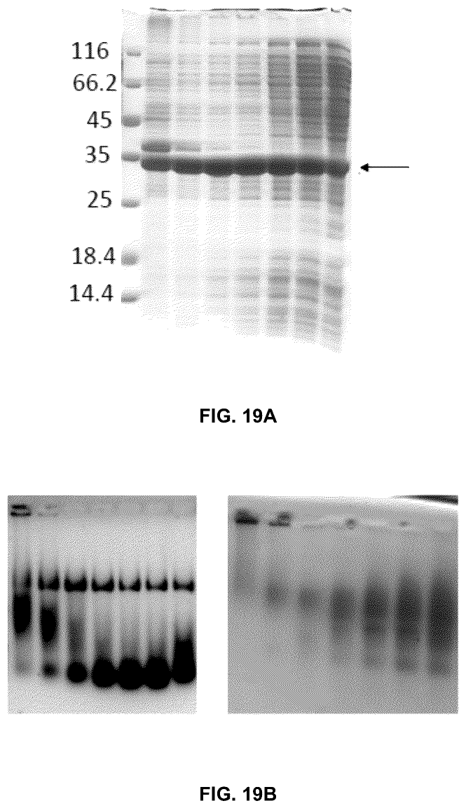

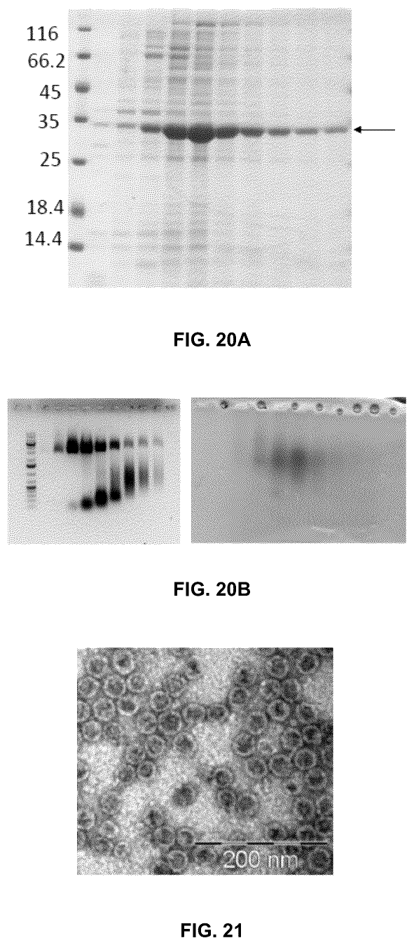

Frozen cells were thawed and resuspended in a lysis buffer (4 ml of buffer per 1 g wet cells). The mixture was sonicated at 24 kHz and 4° C. for 10, with on/off intervals of 0.5 sec. After sonification the lysate was centrifuged for 30 minutes at 10 000 g and pellet discarded. Ammonium sulphate was added to the supernatant to 40% saturation and incubated overnight at +4° C. The mixture was centrifuged for 30 minutes at 10 000 g and the supernatant discarded. The pellet was dissolved in the extraction buffer, centrifuged as described before and obtained supernatant loaded on a Sepharose 4FF (30 ml) column. Proteins were eluted with TBS (Akta Prime Plus, GE Healthcare). The fractions were analyzed by 15% PAGE-SDS and native agarose gel (1% agarose in TAE buffer, stained with 0.05% ethidium bromide) (FIG. 19A, FIG. 19B). Fractions with visible VLP bands in agarose gel were pooled for further ion exchange chromatography. The fractions were loaded on a DEAE Fractogel M (5 ml) column. Bound proteins were eluted with 20 column volumes of linear gradient of 0-1M NaCl in TBS. The fractions were analyzed by 15% PAGE-SDS and native agarose gel (FIG. 20A, FIG. 20B). Fractions containing purified VLPs were dialysed against PBS buffer and concentrated with 15 ml 100 kDa cutoff Amicon concentration filters (Millipore). The presence of VLPs was confirmed by electron microscopy (FIG. 21). The outcome was about 0.6-0.8 mg of purified VLP per gram of wet cells.

### Solutions and Buffers for Example 5

Lysis buffer: 50 mM Tris-HCl pH 8.0, 150 mM NaCl, 0.1% tritonX100, 0.5 M Urea, 1 mM PMSF
Extraction buffer: 20 mM Tris-HCl pH 8.0, 150 mM NaCl, 0.5 M Urea, 0.1% Tween20, 1 mM PMSF
TBS buffer: 20 mM Tris-HCl pH 8.0, 150 mM NaCl
PBS buffer: 137 mM NaCl, 2.7 mM KCl, 10 mM Na₂HPO₄, 1.8 mM KH₂PO₄.

Thus, the generation of fusion proteins with antigenic polypeptides derived from African Swine Fever Virus (ASFV), and in particular from ASFV p12 protein surface exposed peptide in both, N- and C-terminal end, of AP205TD was successfully achieved.

1. A modified virus-like particle of RNA bacteriophage AP205 (AP205 VLP) comprising one or more fusion proteins, wherein said fusion protein comprises, preferably consists of,
(i) an AP205 coat protein dimer, wherein said AP205 coat protein dimer comprises a first AP205 polypeptide and a second AP205 polypeptide, wherein said first AP205 polypeptide is fused at its C-terminus either directly or via an amino acid spacer to the N-terminus of said second AP205 polypeptide, and wherein said first and said second AP205 polypeptide independently comprises
(a) an amino acid sequence of a coat protein of RNA bacteriophage AP205, or
(b) a mutated amino acid sequence, wherein said mutated amino acid sequence (b) and said amino acid sequence of said coat protein of RNA bacteriophage AP205 (a), have a sequence identity of at least 90%, preferably of at least 95%, further preferably of at least 98% and again more preferably of at least 99%; and
(ii) an antigenic polypeptide, wherein said antigenic polypeptide is fused to the N-terminus and/or the C-terminus of said AP205 coat protein dimer either directly or via an amino acid linker.

2. The modified AP205 VLP of claim 1, said first and said second AP205 polypeptide independently comprises (a) an amino acid sequence of a coat protein of RNA bacteriophage AP205, or (b) a mutated amino acid sequence, wherein said mutated amino acid sequence and said amino acid sequence of a coat protein of RNA bacteriophage AP205 have a sequence identity at least 90%, wherein said coat protein of RNA bacteriophage AP205 comprises, preferably consists of, the amino acid sequence of SEQ ID NO: 26.

3. The modified AP205 VLP of claim 1 or claim 2, wherein said amino acid spacer as a length of at most 15 amino acids and is selected from the group consisting of:
(a.) a polyglycine linker (Gly)ₙ of a length of n=2-10, preferably a length of n=2-5; and
(b.) a glycine-serine linker (GS-linker) comprising at least one glycine and at least one serine, wherein preferably said GS linker has an amino acid sequence of (GS)ᵣ(GₛS_{w})ₜ(GS)ᵤ with r=0 or 1, s=1-5, w=0 or 1; t=1-3 and u=0 or 1.

4. The modified AP205 VLP of any one of the preceding claims, wherein said amino acid linker is selected from the group consisting of:
(a.) a polyglycine linker (Gly)ₙ of a length of n=2-10, preferably a length of n=2-5; and
(b.) a glycine-serine linker (GS-linker) comprising at least one glycine and at least one serine, wherein preferably said GS linker has an amino acid sequence of (GS)ᵣ(GₛS_{w})ₜ(GS)ᵤ with r=0 or 1, s=1-5, w=0 or 1; t=1-3 and u=0 or 1; and wherein preferably said glycine-serine linker has a length of at most 15 amino acids;
(c.) an amino acid linker comprising at least one Gly, at least one Ser, and at least one amino acid selected from Thr, Ala, Lys, Asp and Glu, wherein said amino acid sequence has a length of at most 15 amino acids.

5. The modified AP205 VLP of any one of the preceding claims, wherein said AP205 coat protein dimer comprises, preferably consists of, the amino sequence of SEQ ID NO: 7 or an amino acid sequence having a sequence identity of at least 90%, preferably of at least 95%, further preferably of at least 98% and again more preferably of at least 99% with said SEQ ID NO: 7.

6. The modified AP205 VLP of any one of the preceding claims, wherein an antigenic polypeptide is fused directly or via an amino acid linker to the N-terminus of said AP205 coat protein dimer and an antigenic polypeptide is fused directly or via an amino acid linker to the C-terminus of said AP205 coat protein dimer.

7. The modified AP205 VLP of any one of the preceding claims, wherein said modified AP205 VLP consists of said fusion proteins.

8. The modified AP205 VLP of any one of the preceding claims, wherein said antigenic polypeptide is a polypeptide derived from the group consisting of: (a) allergens; (b) viruses; (c) bacteria; (d) parasites; (e) tumors; (f) self-molecules; (g) hormones; (h) growth factors; (i) cytokines; and (j) chemokines.

9. The composition of any one of the preceding claims, wherein said antigenic polypeptide is selected from
(a) IL-17, and wherein preferably said antigenic polypeptide comprises, or preferably consists of, SEQ ID NO:36;
(b) IL-5, and wherein preferably said antigenic polypeptide comprises, or preferably consists of, any one the SEQ ID NO:37 to SEQ ID NO:42;
(c) IL-4, and wherein preferably said antigenic polypeptide comprises, or preferably consists of, any one the SEQ ID NO:43 to SEQ ID NO:45;
(d) IL-13, and wherein preferably said antigenic polypeptide comprises, or preferably consists of SEQ ID NO:49;
(e) IL-1α, and wherein preferably said antigenic polypeptide comprises, or preferably consists of, any one the SEQ ID NO:50 to SEQ ID NO:53;
(f) IL-33, and wherein preferably said antigenic polypeptide comprises, or preferably consists of, any one the SEQ ID NO:54 to SEQ ID NO:58;
(g) IL-25, and wherein preferably said antigenic polypeptide comprises, or preferably consists of, any one the SEQ ID NO:59 to SEQ ID NO:62;
(h) IL-1β, and wherein preferably said antigenic polypeptide comprises, or preferably consists of, any one the SEQ ID NO:63 to SEQ ID NO:67;
(i) IL-12/23;
(j) TNF-α;
(k) IL-31, wherein preferably said antigenic polypeptide comprises, or preferably consists of, any one the SEQ ID NO:68 to SEQ ID NO:71;
(l) thymic stromal lymphopoietin (TLSP), wherein preferably said antigenic polypeptide comprises, or preferably consists of, any one the SEQ ID NO:72 to SEQ ID NO:75;
(m) the dog allergen Can f1 or Can f2;
(n) myostatin, and wherein preferably said antigenic polypeptide comprises, or preferably consists of, SEQ ID NO:87;
(o) an antigenic polypeptide derived from Plasmodium falciparum or Plasmodium Vivax, and wherein preferably said antigenic polypeptide comprises, or preferably consists of, SEQ ID NO:88;
(p) CspZ protein from Borrelia burgdorferi, and wherein preferably said antigenic polypeptide comprises, or preferably consists of, SEQ ID NO:89 or SEQ ID NO:90;
(q) an antigenic polypeptide derived from RSV;
(r) Dengue viral antigenic polypeptide, wherein preferably said Dengue viral antigenic polypeptide comprises, or preferably consists of, SEQ ID NO:91 or SEQ ID NO:92;
(s) an antigenic polypeptide derived from a receptor binding domain (RBD) or a receptor binding motif (RBM) of a coronavirus (CoV), preferably of SARS-CoV-2, and wherein preferably said antigenic polypeptide comprises, or preferably consists of, any one the SEQ ID NO:94 to SEQ ID NO:97;
(t) an antigenic polypeptide derived from African Swine Fever;
(u) calcitonin gene-related peptide (CGRP);
(v) Amylin; and
(w) GnRH.

10. The modified AP205 VLP of any one of the preceding claims, wherein said fusion protein is selected from the group consisting of SEQ ID NO:12, SEQ ID NO:16, SEQ ID NO:21, SEQ ID NO:24, SEQ ID NO:101, SEQ ID NO:106 and SEQ ID NO:110.

11. A pharmaceutical composition comprising:
(a) the AP205 VLP of any one of claims 1 to 10; and
(b) a pharmaceutically acceptable carrier, diluent and/or excipient.

12. The modified AP205 VLP of any one of claims 1 to 10, or the pharmaceutical composition of claim 11 for use in a method of immunization an animal or a human comprising administering the modified AP205 VLP of any one of claims 1 to 10, or the pharmaceutical composition of claim 11 to said animal or human.

13. The modified AP205 VLP of any one of claims 1 to 10, or the pharmaceutical composition of claim 11 for use in a method for the treatment of a disease or disorder in an animal or human, wherein preferably said disease or disorder is selected from the group consisting of autoimmune disease, an inflammatory disease, an infectious disease or a cancer.

14. The modified AP205 VLP or the pharmaceutical composition for use of claim 13, wherein
a) said modified AP205 VLP is the modified AP205 VLP of any one of the claims 9(a) to 9(1), and wherein said disease or disorder is an inflammatory disease in an animal or human, and wherein preferably said inflammatory disease is selected from RA, MS, Psoriasis, ankylosing spondylitis, asthma, Crohns, Colitis, COPD, diabetes, neurodermatitis (allergic dermatitis);
b) said modified AP205 VLP is the modified AP205 VLP of claim 9(m), and wherein said disease or disorder is an allergy in a dog;
c) said modified AP205 VLP is the modified AP205 VLP of claim 9(0) for preventing or treating malaria;
d) said modified AP205 VLP is the modified AP205 VLP of claim 9(p), and wherein said disease or disorder is Lyme borreliosis;
e) said modified AP205 VLP is the modified AP205 VLP of claim 9(q) to 9(t), and wherein said disease or disorder is an infectious disease in an animal or human;
f) said modified AP205 VLP is the modified AP205 VLP of claim 9(u), and wherein said disease or disorder is migraine in an animal or human;
g) said modified AP205 VLP is the modified AP205 VLP of claim 9(v), and wherein said disease, disorder or condition is type II diabetes in an animal or human; or
h) said modified AP205 VLP is the modified AP205 VLP of claims 9(w), and wherein said disease or disorder is to lower testosterone levels in an animal or human.

15. A modified virus-like particle of RNA bacteriophage AP205 (AP205 VLP) comprising one or more AP205 coat protein dimer, wherein said AP205 coat protein dimer comprises a first AP205 polypeptide and a second AP205 polypeptide, wherein said first AP205 polypeptide is fused at its C-terminus either directly or via an amino acid spacer to the N-terminus of said second AP205 polypeptide, and wherein said first and said second AP205 polypeptide independently comprises
(a) an amino acid sequence of a coat protein of RNA bacteriophage AP205, or
(b) a mutated amino acid sequence, wherein said mutated amino acid sequence (b) and said amino acid sequence of said coat protein of RNA bacteriophage AP205 (a), have a sequence identity of at least 90%, preferably of at least 95%, further preferably of at least 98% and again more preferably of at least 99%.


SEQUENCE LISTING (118 sequences)

SEQ ID NO: 1 - DNA, 24 bp, artificial sequence
  upstream primer APncof
tacaccatggcaaataagccaatg

SEQ ID NO: 2 - DNA, 33 bp, artificial sequence
  downstream primer APbamr
tacattaggatccagcagtagtatcagacgata

SEQ ID NO: 3 - DNA, 396 bp, artificial sequence
  template plasmid
atggcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttaa

SEQ ID NO: 4 - DNA, 30 bp, artificial sequence
  upstream primer APbamf
tacaggatccgcaaataagccaatgcaacc

SEQ ID NO: 5 - DNA, 37 bp, artificial sequence
  downstream primer APnher
tacactgcagttagctagcagtagtatcagacgatac

SEQ ID NO: 6 - DNA, 6156 bp, artificial sequence
  pET-Duet1-AP205TD
ggggaattgtgagcggataacaattcccctctagaaataattttgtttaactttaagaaggagatataccatggcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctggatccgcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctagctaacctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 7 - protein, 264 aa, artificial sequence
  AP205TD
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAGSANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAS

SEQ ID NO: 8 - DNA, 711 bp, artificial sequence
  CspZ gene sequence
ctataataaagtttgcttaatagctttataagccccctcaagttctacagcagcacctaaaaaatctgcttcttttttataaaaattattatacctgcttcttgaatttatttccctatcattaaccatatctccaatagttttcacaatacactcaagagctataagatcattttcctctacaaactctttagctttattgacaaattttttagaagcttcaacaaacttactatcaacaaacttagaagatataacatcaaaactctcaatgtaggctaatacaacagattttctaagttttttataagaatcagcaacatgtctggcattagacacatcattatctacagcttggtctaattctatagcaaaatcatcaattaatttacttaaaaacataggtttgtattcttctataatcttttcaagctccttaaacttattaacaattttattgtcttttttaaatatagatatagcttgattaaccttactttgatcagaaaatgtaccttccgaataagtcataatatcattatatgctcctgtacattcgttataaatagcgtctaattttatagaataatacttggccttttcaacaaaaattttaagctcattaatatttctctgatttaatctactaacatcacatgataataaacttattgaaattaacatgtatattgataaaaaactttttttcat

SEQ ID NO: 9 - DNA, 36 bp, artificial sequence
  upstream primer CspZf
tacaccatggcaagaaatattaatgagcttaaaatt

SEQ ID NO: 10 - DNA, 37 bp, artificial sequence
  downstream primer CspZr
cataccatggctaataaagtttgcttaatagctttat

SEQ ID NO: 11 - DNA, 6795 bp, artificial sequence
  pET-Duet1-CspZ-AP205TD
ggggaattgtgagcggataacaattcccctctagaaataattttgtttaactttaagaaggagatataccatggcaagaaatattaatgagcttaaaatttttgttgaaaaggccaagtattattctataaaattagacgctatttataacgaatgtacaggagcatataatgatattatgacttattcggaaggtacattttctgatcaaagtaaggttaatcaagctatatctatatttaaaaaagacaataaaattgttaataagtttaaggagcttgaaaagattatagaagaatacaaacctatgtttttaagtaaattaattgatgattttgctatagaattagaccaagctgtagataatgatgtgtctaatgccagacatgttgctgattcttataaaaaacttagaaaatctgttgtattagcctacattgagagttttgatgttatatcttctaagtttgttgatagtaagtttgttgaagcttctaaaaaatttgtcaataaagctaaagagtttgtagaggaaaatgatcttatagctcttgagtgtattgtgaaaactattggagatatggttaatgatagggaaataaattcaagaagcaggtataataatttttataaaaaagaagcagattttttaggtgctgctgtagaacttgagggggcttataaagctattaagcaaactttattagccatggcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctggatccgcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctagctaacctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 12 - protein, 477 aa, artificial sequence
  CspZ-AP205TD
MARNINELKIFVEKAKYYSIKLDAIYNECTGAYNDIMTYSEGTFSDQSKVNQAISIFKKDNKIVNKFKELEKIIEEYKPMFLSKLIDDFAIELDQAVDNDVSNARHVADSYKKLRKSVVLAYIESFDVISSKFVDSKFVEASKKFVNKAKEFVEENDLIALECIVKTIGDMVNDREINSRSRYNNFYKKEADFLGAAVELEGAYKAIKQTLLAMANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAGSANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAS

SEQ ID NO: 13 - DNA, 33 bp, artificial sequence
  upstream primer CspZf2
tacagctagcagaaatattaatgagcttaaatt

SEQ ID NO: 14 - DNA, 36 bp, artificial sequence
  downstream primer CspZr2
catagctagctaataaagtttgcttaatagctttat

SEQ ID NO: 15 - DNA, 6801 bp, artificial sequence
  pET-Duet1-AP205TD-CspZ
ggggaattgtgagcggataacaattcccctctagaaataattttgtttaactttaagaaggagatataccatggcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctggatccgcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctagcatggcaagaaatattaatgagcttaaaatttttgttgaaaaggccaagtattattctataaaattagacgctatttataacgaatgtacaggagcatataatgatattatgacttattcggaaggtacattttctgatcaaagtaaggttaatcaagctatatctatatttaaaaaagacaataaaattgttaataagtttaaggagcttgaaaagattatagaagaatacaaacctatgtttttaagtaaattaattgatgattttgctatagaattagaccaagctgtagataatgatgtgtctaatgccagacatgttgctgattcttataaaaaacttagaaaatctgttgtattagcctacattgagagttttgatgttatatcttctaagtttgttgatagtaagtttgttgaagcttctaaaaaatttgtcaataaagctaaagagtttgtagaggaaaatgatcttatagctcttgagtgtattgtgaaaactattggagatatggttaatgatagggaaataaattcaagaagcaggtataataatttttataaaaaagaagcagattttttaggtgctgctgtagaacttgagggggcttataaagctattaagcaaactttattagccgctagctaacctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 16 - protein, 479 aa, artificial sequence
  AP205TD-CspZ
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAGSANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTASMARNINELKIFVEKAKYYSIKLDAIYNECTGAYNDIMTYSEGTFSDQSKVNQAISIFKKDNKIVNKFKELEKIIEEYKPMFLSKLIDDFAIELDQAVDNDVSNARHVADSYKKLRKSVVLAYIESFDVISSKFVDSKFVEASKKFVNKAKEFVEENDLIALECIVKTIGDMVNDREINSRSRYNNFYKKEADFLGAAVELEGAYKAIKQTLLAAS

SEQ ID NO: 17 - DNA, 30 bp, artificial sequence
  upstream primer ED3f
gatataccatggataaactgaccctgaaag

SEQ ID NO: 18 - DNA, 33 bp, artificial sequence
  downstream primer ED3r
atttgccatggcaccgctgcccattttgccaat

SEQ ID NO: 19 - DNA, 339 bp, artificial sequence
  ED3 gene of Dengue serotype 1
atggataaactgaccctgaaaggcatgagctatgtgatgtgcaccggcagcttcaaactggaaaaagaagtggcggaaacccagcatggcaccgtgctggtgcaggtgaaatatgaaggcaccgatgcgccgtgcaaaattccgtttagcacccaggatgaaaaaggcgtgacccagaacggccgtctgattaccgcgaatccgattgtgaccgataaagaaaaaccggtgaacattgaaaccgaaccgccgtttggcgaaagctatattgtggtgggtgcgggcgaaaaagcgctgaaactgagctggtttaaaaaaggcagcagcattggcaaaatg

SEQ ID NO: 20 - DNA, 6507 bp, artificial sequence
  pET-Duet1-ED3-AP205TD
ggggaattgtgagcggataacaattcccctctagaaataattttgtttaactttaagaaggagatataccatggataaactgaccctgaaaggcatgagctatgtgatgtgcaccggcagcttcaaactggaaaaagaagtggcggaaacccagcatggcaccgtgctggtgcaggtgaaatatgaaggcaccgatgcgccgtgcaaaattccgtttagcacccaggatgaaaaaggcgtgacccagaacggccgtctgattaccgcgaatccgattgtgaccgataaagaaaaaccggtgaacattgaaaccgaaccgccgtttggcgaaagctatattgtggtgggtgcgggcgaaaaagcgctgaaactgagctggtttaaaaaaggcagcagcattggcaaaatgggcagcggtgccatggcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctggatccgcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctagctaacctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 21 - protein, 381 aa, artificial sequence
  ED3-AP205TD
MDKLTLKGMSYVMCTGSFKLEKEVAETQHGTVLVQVKYEGTDAPCKIPFSTQDEKGVTQNGRLITANPIVTDKEKPVNIETEPPFGESYIVVGAGEKALKLSWFKKGSSIGKMGSGAMANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAGSANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAS

SEQ ID NO: 22 - DNA, 500 bp, artificial sequence
  cIL33 codon-optimized gene
ccatggctagcggtcgtgcgaacgttccgtctatccaggaatactctgcgtctctgtctacctacaacgaccagtctatcaccttcgttttcgaagacggttcttacgaaatctacgttgaagacctgcgtaaaggtcaggaaaaagacaaagttctgttccgttactacgactctcagtctccgtctcacgaaaccggtgacgacgttgacggtcagaccctgctggttaacctgtctccgaccaaagacaaagacttcctgctgcacgcgaacaacgaagaacactctgttgaactgcagaaatgcgaaaaccagctgccggaccaggcgttcttcctgctgcaccgtaaatcttctgaatgcgtttctttcgaatgcaaaaacaacccgggtgttttcatcggtgttaaagacaaccacctggcgctgatcaaagttggtgaccagaccaaagactcttacatcgaaaaaaccatcttcaaactgtcttaactgcag

SEQ ID NO: 23 - DNA, 6645 bp, artificial sequence
  pET-Duet1-AP205TD-cIL33
ggggaattgtgagcggataacaattcccctctagaaataattttgtttaactttaagaaggagatataccatggcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctggatccgcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctagcatggctagcggtcgtgcgaacgttccgtctatccaggaatactctgcgtctctgtctacctacaacgaccagtctatcaccttcgttttcgaagacggttcttacgaaatctacgttgaagacctgcgtaaaggtcaggaaaaagacaaagttctgttccgttactacgactctcagtctccgtctcacgaaaccggtgacgacgttgacggtcagaccctgctggttaacctgtctccgaccaaagacaaagacttcctgctgcacgcgaacaacgaagaacactctgttgaactgcagaaatgcgaaaaccagctgccggaccaggcgttcttcctgctgcaccgtaaatcttctgaatgcgtttctttcgaatgcaaaaacaacccgggtgttttcatcggtgttaaagacaaccacctggcgctgatcaaagttggtgaccagaccaaagactcttacatcgaaaaaaccatcttcaaactgtcttaacctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 24 - protein, 427 aa, artificial sequence
  AP205TD-cIL33
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAGSANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTASMASGRANVPSIQEYSASLSTYNDQSITFVFEDGSYEIYVEDLRKGQEKDKVLFRYYDSQSPSHETGDDVDGQTLLVNLSPTKDKDFLLHANNEEHSVELQKCENQLPDQAFFLLHRKSSECVSFECKNNPGVFIGVKDNHLALIKVGDQTKDSYIEKTIFKLS

SEQ ID NO: 25 - protein, 131 aa, Bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 26 - protein, 130 aa, artificial sequence
  AP205 polypeptide
ANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 27 - protein, 131 aa, artificial sequence
  AP205 polypeptide
ANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAS

SEQ ID NO: 28 - protein, 131 aa, artificial sequence
  P5T mutant CP AP205
MANKTMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 29 - protein, 130 aa, artificial sequence
  AP205 polypeptide
ANKTMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 30 - protein, 131 aa, artificial sequence
  AP205 polypeptide
ANKTMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAS

SEQ ID NO: 31 - protein, 131 aa, artificial sequence
  N14D mutant CP AP205
MANKPMQPITSTADKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 32 - protein, 130 aa, artificial sequence
  AP205 polypeptide
ANKPMQPITSTADKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 33 - protein, 131 aa, artificial sequence
  AP205 polypeptide
ANKPMQPITSTADKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAS

SEQ ID NO: 34 - protein, 514 aa, artificial sequence
  Cry J2
MAMKFIAPMAFVAMQLIIMAAAEDQSAQIMLDSDIEQYLRSNRSLRKVEHSRHDAINIFNVEKYGAVGDGKHDCTEAFSTAWQAACKKPSAMLLVPGNKKFVVNNLFFNGPCQPHFTFKVDGIIAAYQNPASWKNNRIWLQFAKLTGFTLMGKGVIDGQGKQWWAGQCKWVNGREICNDRDRPTAIKFDFSTGLIIQGLKLMNSPEFHLVFGNCEGVKIIGISITAPRDSPNTDGIDIFASKNFHLQKNTIGTGDDCVAIGTGSSNIVIEDLICGPGHGISIGSLGRENSRAEVSYVHVNGAKFIDTQNGLRIKTWQGGSGMASHIIYENVEMINSENPILINQFYCTSASACQNQRSAVQIQDVTYKNIRGTSATAAAIQLKCSDSMPCKNINLSDISLKLTSGKIASCLNDNANGYFSGHVIPACKNLSPSAKRKESKSHKHPKTVMVENMGAYDKGNRTRILLGSRPPNCTNKCHGCSPCKAKLVIVHRIMPQEYYPQRWMCSCHGKIYHP

SEQ ID NO: 35 - protein, 371 aa, artificial sequence
  Amb a1
AEDLQEILPVNETRRLTTSGAYNIIDGCWRGKADWAENRKALADCAQGFGKGTVGGKDGDIYTVTSDLDDDVANPKEGTLRFGAAQNRPLWIIFERDMVIRLDKEMVVNSDKTIDGRGAKVEIINAGFTLNGVKNVIIHNINMHDVKVNPGGLIKSNDGPAAPRAGSDGDAISISGSSQIWIDHCSLSKSVDGLVDAKLGTTRLTVSNSLFTQHQFVLLFGAGDENIEDRGMLATVAFNTFTDNVDQRMPRCRHGFFQVVNNNYDKWGSYAIGGSASPTILSQGNRFCAPDERSKKNVLGRHGEAAAESMKWNWRTNKDVLENGAIFVASGVDPVLTPEQSAGMIPAEPGESALSLTSSAGVLSCQPGAPC

SEQ ID NO: 36 - protein, 132 aa, homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 37 - protein, 115 aa, homo sapiens
IPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 38 - protein, 114 aa, artificial sequence
  canine IL-5
AVENPMNRLVAETLTLLSTHRTWLIGDGNLMIPTPENKNHQLCIKEVFQGIDTLKNQTAHGEAVDKLFQNLSLIKEHIERQKKRCAGERWRVTKFLDYLQVFLGVINTEWTPES

SEQ ID NO: 39 - protein, 114 aa, artificial sequence
  feline IL-5
AVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMES

SEQ ID NO: 40 - protein, 115 aa, artificial sequence
  feline IL-5
IAVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMES

SEQ ID NO: 41 - protein, 109 aa, artificial sequence
  feline IL-5
IAVQSPMNRLVAETLALLSTHRTLLIGDGNLMIPTPEHNNHQLCIEEVFQGIDTLKNRTVPGDAVEKLFRNLSLIKEHIDRQKKNFGGKKWKVKNFLNYLQFFLGLLNT

SEQ ID NO: 42 - protein, 115 aa, artificial sequence
  equine IL-5
LAVESPMNRLVAETLTLLSTHRTLLIGDGNLMIPTPEHKNHQLCIEEVFQGIDTLKNQTVQGDAVAKLFQNLSLIKGYIDLQKKKCGGERWRVKQFLDYLQEFLGVINTEWTIEG

SEQ ID NO: 43 - protein, 129 aa, homo sapiens [sequence omitted]

SEQ ID NO: 44 - protein, 109 aa, artificial sequence
  canine IL-4
GHNFNITIKEIIKMLNILTARNDSCMELTVKDVFTAPKNTSDKEIFCRAATVLRQIYTHNCSNRYLRGLYRNLSSMANKTCSMNEIKKSTLKDFLERLKVIMQKKYYRH

SEQ ID NO: 45 - protein, 109 aa, artificial sequence
  feline IL-4
QNFNNTLKEIIKTLNILTARNDSCMELTVMDVLAAPKNTSDKEIFCRATTVLRQIYTHHNCSTKFLKGLDRNLSSMANRTCSVNEVKKCTLKDFLERLKAIMQKKYSKH

SEQ ID NO: 46 - protein, 122 aa, homo sapiens
LTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 47 - protein, 113 aa, artificial sequence
  canine IL-13 mature protein
SPSPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFR

SEQ ID NO: 48 - protein, 113 aa, artificial sequence
  feline IL-13 mature protein
SPGPHSRRELKELIEELVNITQNQVSLCNGSMVWSVNLTTGMQYCAALESLINVSDCTAIQRTQRMLKALCTQKPSAGQTASERSRDTKIEVIQLVKNLLNHLRRNFRHGNFK

SEQ ID NO: 49 - protein, 121 aa, artificial sequence
  equine IL-13 mature protein
CLGGLASPAPLPSSMALKELIKELVNITQNQAPLCNGSMVWSVNLTADTYCRALESLSNVSTCSAIQNTRKMLTKLCPHQLSAGQVSSERARDTKIEVIVLVKDLLKNLRKIFHGGKHVDA

SEQ ID NO: 50 - protein, 159 aa, homo sapiens
SAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 51 - protein, 157 aa, artificial sequence
  canine IL-1alpha
SVAYNFHNNEKYNYIRIIKSQFILNDNLNQSIVRQTGGNYLMTAALQNLDDAVKFDMGAYTSEDSKLPVTLRISKTRLFVSAQNEDEPVLLKEMPETPKTIRDETNLLFFWERHGSKHYFKSVAQPKLFIATQERKLVHMARGQPSITDFRLLETQP

SEQ ID NO: 52 - protein, 158 aa, artificial sequence
  feline IL-1alpha
SVAPNFYSSEKYNYQKIIKSQFILNDNLSQSVIRKAGGKYLAAAALQNLDDAVKFDMGAYTSKEDSKLPVTLRISKTRLFVSAQNEDEPVLLKEMPETPKTIRDETNLLFFWERHGSKNYFKSVAHPKLFIATQEEQLVHMARGLPSVTDFQILETQS

SEQ ID NO: 53 - protein, 158 aa, artificial sequence
  equine IL-1alpha
SVHYNFQSNTKYNFMRIVNHQCTLNDALNQSVIRDTSGQYLATAALNNLDDAVKFDMGAYTSEEDSQLPVTLRISKTRLFVSAQNEDEPVLLKEMPDTPKTIKDETNLLFFWERHGSKNYFKSVAHPKLFIATKQGKLVHMARGQPSITDFQILDNQF

SEQ ID NO: 54 - protein, 175 aa, homo sapiens
FGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 55 - protein, 151 aa, artificial sequence
  canine IL-33
EYSASLSTYNDQSITFVFEDGSYEIYVEDLRKGQEKDKVLFRYYDSQSPSHETGDDVDGQTLLVNLSPTKDKDFLLHANNEEHSVELQKCENQLPDQAFFLLHRKSSECVSFECKNNPGVFIGVKDNHLALIKVGDQTKDSYIEKTIFKLS

SEQ ID NO: 56 - protein, 163 aa, artificial sequence
  canine IL-33
MASGRANVPSIQEYSASLSTYNDQSITFVFEDGSYEIYVEDLRKGQEKDKVLFRYYDSQSPSHETGDDVDGQTLLVNLSPTKDKDFLLHANNEEHSVELQKCENQLPDQAFFLLHRKSSECVSFECKNNPGVFIGVKDNHLALIKVGDQTKDSYIEKTIFKLS

SEQ ID NO: 57 - protein, 170 aa, artificial sequence
  feline IL-33
FGVPMVQKCFGTTDVPSIQEYSASLSTYNDQSITFVFEDGSYEIYVEDLGKDQEKDKVLFRYYDFQSPSRETGDADDGHTLLVNLSPTKDKDFLLHANNKEHSVELQKCKKPLPDQAFFRLHRKSSKCVSFECKNDPGVFIGVKDNHLALIKVEDQTEYFSTENIIFKLS

SEQ ID NO: 58 - protein, 170 aa, artificial sequence
  equine IL-33
FDVPMIQRCARSTAGPIFEEDFASLSTYNDQSISFVIEDGSYEIYIEDLSGQQEKDPVLFRCYPSQCSSSETAGGADGQKLMVNLSPKKYKDFLLHANNKEHSVELQKCENPFPDQAFFLMHKTSSRCVSFECKNNPGVFIGVKDKHLALIKVGEQTEPSSRQNIIFKLS

SEQ ID NO: 59 - protein, 145 aa, homo sapiens
YSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 60 - protein, 147 aa, artificial sequence
  canine IL-25
KDCTHWPNCCPSKRQDPTHEWLKRDTVLKFPGETTSLTHHPESCKASEDGPLNSRSISPWKYELDRDLNRLPQDLYHARCLCQHCVSLQTGSHMDPLGNSELLYHNQTVFYRRPCPGEQGAPDGYCLEQRLYRVSLACVCVRPRVMA

SEQ ID NO: 61 - protein, 147 aa, artificial sequence
  feline IL-25
KDCIHWSSCCPSKGQNPTHEWLTENTVLMTPPESASLIHSLESCRASEDGPLNSRSISPWRYELDRDLNRLPQDLYHARCLCPHCVSLQTGSHMDPLGNSELLYHNQTVFYRRPCPGEQGTRDGYCLEQRLYRVSLACVCVRPRVMA

SEQ ID NO: 62 - protein, 147 aa, artificial sequence
  equine IL-25
KECTHWPSCCPKKGQDPIEEWLKWSTAHVPPPETANLAHHPESCRASEDGPLNSRSISPWRYELDRDLNRLPQDLYHARCLCPHCVSLQTGSHMDPLGNSELLYHNQTVFYRRPCPGKRGAHDGYCLERRLYRVSLACVCVRPRVMA

SEQ ID NO: 63 - protein, 153 aa, homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 64 - protein, 152 aa, artificial sequence
  canine IL-1b
AAMQSVDCKLQDISHKYLVLSNSYELRALHLNGENVNKQVVFHMSFVHGDESNNKIPVVLGIKQKNLYLSCVMKDGKPTLQLEKVDPKVYPKRKMEKRFVFNKIEIKNTVEFESSQYPNWYISTSQVEGMPVFLGNTRGGQDITDFTMEFSS

SEQ ID NO: 65 - protein, 152 aa, artificial sequence
  canine IL1b
AAMQSVDCKLQGISHKYLVLSNSYELRALHLNGENVNKQVVFHMSFVHGDESNNKIPVVLGIKQKNLYLSCVMKDGKPTLQLEKVDPKVYPKRKMEKRFVFNKIEIKNTVEFESSQYPNWYISTSQVEGMPVFLGNTRGGQDITDFTMEFSS

SEQ ID NO: 66 - protein, 152 aa, artificial sequence
  canine IL-1b
AALPSVDCTLQDINHKYLVLSNSYELRALHLNGENVNKQVVFHMSFVHGDESNYKIPVVLGIKQKNLYLSCVMKDGKPTLQLEKVDPKVYPKRKMEKRFVFNKIEIKNTVEFESSQYPNWYISTSQVEGMPVFLGNTKGGQDITDFTMEFSS

SEQ ID NO: 67 - protein, 152 aa, artificial sequence
  feline IL-1b
AAIQSQDYTFRDISQKSLVLSGSYELRALHLNGQNMNQQVVFRMSFVHGEENSKKIPVVLCIKKNNLYLSCVMKDGKPTLQLEMLDPKVYPKKKMEKRFVFNKTEIKGNVEFESSQFPNWYISTSQAEEMPVFLGNTKGGQDITDFIMESAS

SEQ ID NO: 68 - protein, 141 aa, homo sapiens [sequence omitted]

SEQ ID NO: 69 - protein, 136 aa, artificial sequence
  canine IL-31
SHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQ

SEQ ID NO: 70 - protein, 136 aa, artificial sequence
  feline IL-31
SHMAPAHRLQPSDVRKIILELRPMSKGLLQDYLKKEIGLPESNHSSLPCLSSDSQLPHINGSAILPYFRAIRPLSDKNTIDKIIEQLDKLKFQREPEAKVSMPADNFERKNFILAVLQQFSACLEHVLQSLNSGPQ

SEQ ID NO: 71 - protein, 130 aa, artificial sequence
  equine IL-31
SHTGPIYQLQPKEIQAIIVELQNLSKKLLDDYLNKEKGVQKFDSDLPSCFTSDSQAPGNINSSAILPYFKAISPSLNNDKSLYIIEQLDKLNFQNAPETEVSMPTDNFERKRFILTILRWFSNCLEHRAQ

SEQ ID NO: 72 - protein, 131 aa, homo sapiens
YDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 73 - protein, 127 aa, artificial sequence
  canine TLSP
YNFIDCDFEKIRWKYQEVIYQALEKYMDGTRSTEFSHPVYCADPPDCLARIERLTLHRIRGCASGAREAFAEGTVAALAAECPGYAAAPINNTQAKKKRKKRGVTTNKCREQVAHLIGLWRRFSRIS

SEQ ID NO: 74 - protein, 127 aa, artificial sequence
  feline TLSP
YNFTDCDFGKIRKKYQEVIYQALNKYMNGVKSTEFNNYIYCEDPPDCLAKIDLITFYPTHGCTTLAKEIFAIRTNATLTLQCPGYSGTQINNTQAKKKRKKRQVTTNKCREQVSHLMELWRRFSRIS

SEQ ID NO: 75 - protein, 125 aa, artificial sequence
  equine TLSP
YDFTDCNFAKIEEEYQNVIFLALKEYMNGIKSTQFNHTVYCGDRPSCLTEIERLTFKSSQCLSLAKKIVTATNATLNSHCPGHSGIQINNTQAMKKRKKREVTTNKCLKQVSNLIELWRYFSRSQ

SEQ ID NO: 76 - protein, 42 aa, homo sapiens [sequence omitted]

SEQ ID NO: 77 - protein, 6 aa, artificial sequence
  Abeta 1-6
DAEFRH

SEQ ID NO: 78 - protein, 7 aa, artificial sequence
  Abeta 1-7
DAEFRHD

SEQ ID NO: 79 - protein, 4 aa, artificial sequence
  Abeta 3-6
EFRH

SEQ ID NO: 80 - protein, 5 aa, artificial sequence
  Abeta 1-5
DAEFR

SEQ ID NO: 81 - protein, 5 aa, artificial sequence
  Abeta 2-6
AEFRH

SEQ ID NO: 82 - protein, 5 aa, artificial sequence
  Abeta 3-7
EFRHD

SEQ ID NO: 83 - protein, 10 aa, artificial sequence
  alpha-synuclein peptide egy
EGYQDYEPEA

SEQ ID NO: 84 - protein, 8 aa, artificial sequence
  alpha-synuclein petide kne
KNEEGAPQ

SEQ ID NO: 85 - protein, 8 aa, artificial sequence
  alpha-synuclein petide mdv
MDVFMKGL

SEQ ID NO: 86 - protein, 7 aa, artificial sequence
  alpha-syn kne*
KNEEGAP

SEQ ID NO: 87 - protein, 95 aa, artificial sequence
  cow myostatin
CCRYPLTVDFEAFGWDWIIAPKRYKANYCSGECEFVFLQKYPHTHLVHQANPRGSAGPCCTPTKMSPINMLYFNGEGQIIYGKIPAMVVDRCGCS

SEQ ID NO: 88 - protein, 165 aa, artificial sequence
  plasmodium vivax
DRADGQPAGDRADGQPAGDRAAGQPAGDRAAGQPAGDRADGQPAGDRADGQPAGAPGANQEGGAAAPGANQEGGAAAPGANQEGGAAAPGANQEGGAAAPGANQEGGAAAPGANQEGGAAANGAGNQPGANGAGNQPGANGAGNQPGANGAGNQPGANGAGNQPG

SEQ ID NO: 89 - protein, 213 aa, artificial sequence
  CspZ
MARNINELKIFVEKAKYYSIKLDAIYNECTGAYNDIMTYSEGTFSDQSKVNQAISIFKKDNKIVNKFKELEKIIEEYKPMFLSKLIDDFAIELDQAVDNDVSNARHVADSYKKLRKSVVLAYIESFDVISSKFVDSKFVEASKKFVNKAKEFVEENDLIALECIVKTIGDMVNDREINSRSRYNNFYKKEADFLGAAVELEGAYKAIKQTLLA

SEQ ID NO: 90 - protein, 215 aa, artificial sequence
  CspZ
MARNINELKIFVEKAKYYSIKLDAIYNECTGAYNDIMTYSEGTFSDQSKVNQAISIFKKDNKIVNKFKELEKIIEEYKPMFLSKLIDDFAIELDQAVDNDVSNARHVADSYKKLRKSVVLAYIESFDVISSKFVDSKFVEASKKFVNKAKEFVEENDLIALECIVKTIGDMVNDREINSRSRYNNFYKKEADFLGAAVELEGAYKAIKQTLLAAS

SEQ ID NO: 91 - protein, 117 aa, artificial sequence
  ED3 of serotype 1
MDKLTLKGMSYVMCTGSFKLEKEVAETQHGTVLVQVKYEGTDAPCKIPFSTQDEKGVTQNGRLITANPIVTDKEKPVNIETEPPFGESYIVVGAGEKALKLSWFKKGSSIGKMGSGA

SEQ ID NO: 92 - protein, 113 aa, artificial sequence
  ED3 of serotype 1
MDKLTLKGMSYVMCTGSFKLEKEVAETQHGTVLVQVKYEGTDAPCKIPFSTQDEKGVTQNGRLITANPIVTDKEKPVNIETEPPFGESYIVVGAGEKALKLSWFKKGSSIGKM

SEQ ID NO: 93 - protein, 24 aa, artificial sequence
  Influenza M2 peptide
SLLTEVETPIRNEWGCRCNDSSDG

SEQ ID NO: 94 - protein, 211 aa, artificial sequence
  RBD SARS-CoV2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 95 - protein, 72 aa, artificial sequence
  RBM SARS-CoV2
NSNNLDSKVGGNYNYLYRLFRKSNLKPFERDISTEIYQAGSTPCNGVEGFNCYFPLQSYGFQPTNGVGYQPY

SEQ ID NO: 96 - protein, 211 aa, artificial sequence
  RBD MERS
QAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPK

SEQ ID NO: 97 - protein, 83 aa, artificial sequence
  RBM MERS
VPHNLTTITKPLKYSYINKCSRLLSDDRTEVPQLVNANQYSPCVSIVPSTVWEDGDYYRKQLSPLEGGGWLVASGSTVAMTEQ

SEQ ID NO: 98 - DNA, 633 bp, artificial sequence
  pUCIDT-SARS-CoV-2-RBD
aacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagccgtatcgcgtggtggtgctgagctttgaactgctgcatgcgccggcgaccgtgtgcggcccgaaaaaaagcaccaacctggtgaaaaacaaatgcgtgaacttt

SEQ ID NO: 99 - DNA, 37 bp, artificial sequence
  primer F
tctgatactactgctagcggatccaacagcaacaacc

SEQ ID NO: 100 - DNA, 6388 bp, artificial sequence
  pETDuet-1-AP205-RBM
ggggaattgtgagcggataacaattcccctctagaaataattttgtttaactttaagaaggagatataccatggcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctggatccgcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctagcggatccaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagccgtatactagtcatcaccatcaccatcactaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 101 - protein, 338 aa, artificial sequence
  AP205-RBM
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAGSANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTASGSNSNNLDSKVGGNYNYLYRLFRKSNLKPFERDISTEIYQAGSTPCNGVEGFNCYFPLQSYGFQPTNGVGYQPY

SEQ ID NO: 102 - protein, 12 aa, artificial sequence
  ASFV p12
MALDGSSGGGSN

SEQ ID NO: 103 - DNA, 55 bp, artificial sequence
  ASFV p12 forward
atataccatggcgctggatggtagttcgggtggagggtcgaatgccatggcaaat

SEQ ID NO: 104 - DNA, 55 bp, artificial sequence
  ASFV p12 reverse
atttgccatggcattcgaccctccacccgaactaccatccagcgccatggtatat

SEQ ID NO: 105 - DNA, 6191 bp, artificial sequence
  pET-Duet1-np12-AP205TD
ggggaattgtgagcggataacaattcccctctagaaataattttgtttaactttaagaaggagatataccatggcgctggatggtagttcgggtggagggtcgaatgccatggcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctggatccgcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctagcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 106 - protein, 277 aa, artificial sequence
  np12-AP205TD
MALDGSSGGGSNAMANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAGSANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAS

SEQ ID NO: 107 - DNA, 62 bp, artificial sequence
  ASFV p12 forward C
ctactgctagcatggcgctggatggtagttcgggtggagggtcgaattaacctgcaggtcga

SEQ ID NO: 108 - DNA, 62 bp, artificial sequence
  ASFV p12 reverse C
tcgacctgcaggttaattcgaccctccacccgaactaccatccagcgccatgctagcagtag

SEQ ID NO: 109 - DNA, 6231 bp, artificial sequence
  pET-Duet1-np12-AP205TD-cp12
ggggaattgtgagcggataacaattcccctctagaaataattttgtttaactttaagaaggagatataccatggcgctggatggtagttcgggtggagggtcgaatgccatggcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctggatccgcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgctagcatggcgctggatggtagttcgggtggagggtcgaattaacctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 110 - protein, 289 aa, artificial sequence
  np12-AP205TD-cp12
MALDGSSGGGSNAMANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAGSANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTASMALDGSSGGGSN

SEQ ID NO: 111 - protein, 263 aa, artificial sequence
  AP205TD*
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAGSANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 112 - protein, 154 aa, artificial sequence
  canine IL-33
SIQEYSASLSTYNDQSITFVFEDGSYEIYVEDLRKGQEKDKVLFRYYDSQSPSHETGDDVDGQTLLVNLSPTKDKDFLLHANNEEHSVELQKCENQLPDQAFFLLHRKSSECVSFECKNNPGVFIGVKDNHLALIKVGDQTKDSYIEKTIFKLS

SEQ ID NO: 113 - protein, 160 aa, artificial sequence
  canine IL-33
GRANVPSIQEYSASLSTYNDQSITFVFEDGSYEIYVEDLRKGQEKDKVLFRYYDSQSPSHETGDDVDGQTLLVNLSPTKDKDFLLHANNEEHSVELQKCENQLPDQAFFLLHRKSSECVSFECKNNPGVFIGVKDNHLALIKVGDQTKDSYIEKTIFKLS

SEQ ID NO: 114 - protein, 10 aa, artificial sequence
  GnRH
EHWSYGLRPG

SEQ ID NO: 115 - protein, 10 aa, artificial sequence
  GnRH
QHWSYGLRPG

SEQ ID NO: 116 - protein, 8 aa, artificial sequence
  Angio 2
DRVYIHPF

SEQ ID NO: 117 - protein, 7 aa, artificial sequence
  Angio 2*
DRVYIHP

SEQ ID NO: 118 - protein, 10 aa, artificial sequence
  Angio 1
DRVYIHPFHL